(19) United States
(12) Patent Application Publication
(10) Pub. No.: US 2025/0059193 A1
(43) Pub. Date: Feb. 20, 2025

(54) POLYMORPHIC AND SALT FORMS OF (1S,3S)-N1-(5-(PENTAN-3-YL)PYRAZOLO[1,5-A]PYRIMIDIN-7-YL) CYCLOPENTANE-1,3-DIAMINE

(71) Applicant: Kronos Bio, Inc., San Mateo, CA (US)

(72) Inventors: Pasit Phiasivongsa, San Mateo, CA (US); Xinnan Zhang, San Mateo, CA (US); Geoffrey Yeh, San Mateo, CA (US); Rene Steendam, San Mateo, CA (US); David Freeman, San Mateo, CA (US); Stefan Proniuk, San Mateo, CA (US); Bernhard Paul, San Mateo, CA (US)

(21) Appl. No.: 18/712,666

(22) PCT Filed: Nov. 22, 2022
(86) PCT No.: PCT/US2022/050763

§ 371 Date: May 22, 2024

Related U.S. Application Data
(60) Provisional application No. 63/283,073, filed on Nov. 24, 2021.

(51) Int. Cl. C07D 487/04; A61K 31/519; C07C 55/07; C07C 57/15; C07C 59/265; C07C 317/12; C07C 317/14

(52) U.S. Cl. CPC C07D 487/04; A61K 31/519; C07C 55/07; C07C 57/15; C07C 59/265; C07C 317/12; C07C 317/14; C07C 2601/16; C07C 2602/10; C07C 2602/42

(57) ABSTRACT

Provided are polymorphic and salt forms of (1S,3S)—N¹—(5-(pentan-3-yl)pyrazolo[1,5-α]pyrimidin-7-yl) cyclopentane-1,3-diamine. Provided are polymorphic forms of the free base of the compound along with polymorphic forms of salts of the compound, including tosylate salts, oxalate salts, fumarate salts, camsylate salts, citrate salts, hydrochloride salts, and naphthalene-1,5-disulfonic acid salts. Such forms have been characterized by X-ray diffraction (XRD), differential scanning calorimetry (DSC), and thermogravimetric analysis coupled to mass spectrometry (TGA-MS). Such polymorphic and salt forms can have desirable solubility, dissolution, and pharmacokinetic properties.

### INTRODUCTION

(1S,3S)—N¹—(5-(pentan-3-yl)pyrazolo[1,5-α]pyrimidin-7-yl)cyclopentane-1,3-diamine is a pharmaceutically active compound that has been studied for various uses, such as for the treatment of cancer. As used herein, the term “Compound A” is used to refer to both the free base and salt forms of (1S,3S)—N¹—(5-(pentan-3-yl)pyrazolo[1,5-α]pyrimidin-7-yl)cyclopentane-1,3diamine. The free base of Compound A has the CAS number of 2416873-83-9 and structure of formula (I):

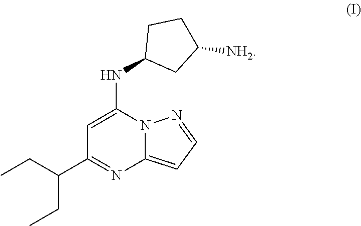 [SMILES: CCC(CC)c1cc(N[C@H]2CC[C@H](N)C2)n2nccc2n1]

For example, Compound A has been reported to be a selective inhibitor of cyclin-dependent kinase 9 (CDK9) that has shown anti-tumor activity with in vitro and in vivo cancer models (Richters et al, Cell Chemical Biology, 2021, 28, 2, 134, doi: 10.1016/j.chembiol.2020.10.001). In fact, it has been reported that inhibiting CDK9, e.g. with Compound A, can impact multiple survival pathways in cancers and could be a therapeutic approach for glioblastoma, which is a cancer subtype that is resistant to many existing treatments (Ranjan et al, Cancers, 2021, 13, 12, 3039, doi: 10.3390/cancers13123039).

The cyclin-dependent kinase (CDK) family of proteins are key regulators of the cell cycle and gene transcription. The cell cycle is a regulatory cellular mechanism for the timing of cell growth and division. The cell cycle is a multi-pronged process that directs cellular proliferation through a series of checkpoints that correct for DNA damage, genetic derangements, and other errors (Nonhuman Primates in Biomedical Research 20, Second Edition, 2012). Each stage is controlled by a combination of cyclins and CDKs, where the CDKs phosphorylate a specific set of cyclins to trigger entry into the next stage of the cell cycle (Merri Lynn Casem, “Case Studies in Cell Biology”, Chapter 13—Cell Cycle, 2016, doi: 10.1016/B978-0-12-801394-6.00013-0). Accumulation of cyclin proteins through regulation of cyclin mRNA transcription function as “biological switches” to turn CDKs on and off and move the cell from one stage to the next (Id). CDKs 1, 2, 3, 4 and 6 regulate time of the cell division cycle while CDK 7 and CDK 9 regulate the activity of transcription through regulation of RNA polymerase II via phosphorylation of its carboxy terminal domain (Lucking et al, ChemMedChem, 2017, 12, 1776, doi: 10.1002/cmdc.201700447).

CDK9 controls the transcriptional activity of key oncogenic proteins such as AR, MYC, MCL-1, and BCL-2 and stimulates pro-inflammatory transcription factors such as NFKB and STAT3 (Gregory et al, Leukemia, 2015, 29, 1437, doi: 10.1038/leu.2015.10; Krystof et al, Current Pharmaceutical Design, 18, 20, 2883, doi: 10.2174/138161212800672750). CDK9 forms a heterodimer with one of four cyclin partners (cyclin T1, cyclin K, cyclin T2a, or cyclin T2b) called positive transcription elongation factor (PTEFb). RNA polymerase II pauses mRNA transcription after 20-40 nucleotides along the DNA template due to interaction of negative elongation factors which serve as a major regulatory control mechanism for transcription of rapidly induced genes. PTEFb overcomes pausing of RNA polymerase II by phosphorylation of the carboxy terminal domain of RNA polymerase II, and inactivation of negative elongation factors. Compounds targeting CDK9 and PTEFb are currently undergoing clinical study. The enzymatic activity of CDK9 is important for stimulating transcription elongation of most protein coding genes (Krystof et al, Current Pharmaceutical Design, 18, 20, 2883, doi: 10.2174/138161212800672750).

### SUMMARY

Provided are polymorphic and salt forms of Compound A, which is also known as (1S,3S)—N¹—(5-(pentan-3-yl)pyrazolo[1,5-α]pyrimidin-7-yl) cyclopentane-1,3-diamine. Provided are polymorphic forms of the free base of Compound A along with polymorphic forms of salts of Compound A, including tosylate salts, oxalate salts, fumarate salts, camsylate salts, citrate salts, hydrochloride salts, and naphthalene-1,5-disulfonic acid salts. Such forms have been characterized by X-ray diffraction (XRD), differential scanning calorimetry (DSC), and thermogravimetric analysis coupled to mass spectrometry (TGA-MS). Such polymorphic and salt forms can have desirable solubility, dissolution, and pharmacokinetic properties.

In some embodiments, polymorphic forms of the free base of Compound A are provided by this disclosure. In one aspect, polymorphic forms I, II, and III of the free base of Compound A are provided. In some cases, form III is amorphous. In some cases, such forms are characterized by X-ray diffraction (XRD) patterns, differential scanning calorimetry (DSC) thermograms, and thermogravimetric analysis coupled to mass spectrometry (TGA-MS) spectrum substantially as shown in FIGS. 1-6, 7A-7C, 8A-8C, and 9A-9C.

In some embodiments, polymorphic forms of a tosylate salt of Compound A are provided. In one aspect, forms I, II, III, and IV of a tosylate salt of Compound A are provided. In some cases, such forms are characterized by XRD, DSC, and TGA-MS spectra substantially as shown in FIGS. 10-17.

In some embodiments, polymorphic forms of an oxalate salt of Compound A are provided. In one aspect, forms I, II, and III of an oxalate salt of Compound A are provided. In some cases, such forms are characterized by XRD, DSC, and TGA-MS spectra substantially as shown in FIGS. 19-27.

In some embodiments, polymorphic forms of a fumarate salt of Compound A are provided. In one aspect, forms I, II, III, IV, and V of an oxalate salt of Compound A are provided. In some cases, such forms are characterized by XRD, DSC, and TGA-MS spectra substantially as shown in FIGS. 29-35.

In some embodiments, polymorphic forms of a camsylate salt of Compound A are provided. In one aspect, forms I, II, and III of a camsylate salt of Compound A are provided. In some cases, such forms are characterized by XRD, DSC, and TGA-MS spectra substantially as shown in FIGS. 37-42.

In some embodiments, polymorphic forms of a citrate salt of Compound A are provided. In one aspect, form I of a citrate salt of Compound A are provided. In some cases, such forms are characterized by XRD, DSC, and TGA-MS spectra substantially as shown in FIGS. 44-46

In some embodiments, polymorphic forms of a hydrochloride salt of Compound A are provided. In one aspect, forms I, II, and III of an oxalate salt of Compound A are provided. In some cases, such forms are characterized by XRD, DSC, and TGA-MS spectra substantially as shown in FIGS. 47-53.

In some embodiments, polymorphic forms of a naphthalene-1,5-disulfonic acid salt of Compound A are provided. In one aspect, form I of a naphthalene-1,5-disulfonic acid salt of Compound A is provided. In some cases, such forms are characterized by XRD, DSC, and TGA-MS spectra substantially as shown in FIG. 54.

Methods of synthesizing a polymorph of formula (I) as described herein are provided. In some cases the method is a method of synthesizing a polymorph of a tosylate salt, an oxalate salt, a fumarate salt, a camsylate salt, a citrate salt, a hydrochloride salt, or a naphthalene-1,5-disulfonic acid salt of formula (I). Such methods can include: (i) contacting a free base of Compound A with a solvent and a compound comprising a tosylate moiety, an oxalate moiety, a fumarate moiety, a camsylate moiety, a citrate moiety, a chloride moiety, or a naphthalene-1,5-disulfonic acid moiety, thereby generating a composition comprising the solvent and a salt of formula (I); and (ii) evaporating the solvent to generate a solid product.

Pharmaceutical compositions are provided that comprise a polymorphic form of formula (I) as described herein and a pharmaceutically acceptable carrier.

Kits are provided that comprise: (i) a polymorphic form of formula (I) as described herein or a pharmaceutical composition comprising such a polymorphic form; and (ii) instructions for use of the polymorphic form or the pharmaceutical composition.

Methods of treating a condition in a subject provided, wherein the method comprises administering to the subject a polymorphic form of formula (I) as described herein or a pharmaceutical composition comprising such a polymorphic form. In some cases the subject is a human. In some cases the condition is cancer or an autoimmune disease.

### BRIEF DESCRIPTION OF THE DRAWINGS

FIG. 1 shows an X-ray diffraction (XRD) pattern of the free base, form I.

FIG. 2 shows a differential scanning calorimetry (DSC) thermogram of the free base, form I.

FIG. 3 shows a thermogravimetric analysis coupled to mass spectrometry (TGA-MS) characterization of the free base, form I.

FIG. 4 shows an XRD pattern of the free base, form II.

FIG. 5 shows a DSC thermogram of the free base, form II.

FIG. 6 shows a TGA-MS characterization of the free base, form II.

FIG. 7A shows an XRD pattern of the free base, form III, experiment 1.

FIG. 7B shows an DSC thermogram of the free base, form III, experiment 1.

FIG. 7C shows an TGA-MS characterization of the free base, form III, experiment 1.

FIG. 7D shows XRD patterns of a free base upon slow evaporation of an ethanol solution (bottom) and 45 minutes later (top).

FIG. 8A shows an XRD pattern of the free base, form III, experiment 2.

FIG. 8B shows an DSC thermogram of the free base, form III, experiment 2.

FIG. 8C shows an TGA-MS characterization of the free base, form III, experiment 3.

FIG. 9A shows an XRD pattern of the free base, form III, experiment 3.

FIG. 9B shows an DSC thermogram of the free base, form III, experiment 3.

FIG. 9C shows an TGA-MS characterization of the free base, form III, experiment 3.

FIG. 10 shows an XRD pattern of a tosylate salt, form I.

FIG. 11 shows a DSC thermogram of a tosylate salt, form I.

FIG. 12 shows a TGA-MS characterization of a tosylate salt, form I.

FIG. 13 shows a XRD pattern of a tosylate salt, form II.

FIG. 14 shows an XRD pattern of a tosylate salt, form III.

FIG. 15 shows a DSC thermogram of a tosylate salt, form III.

FIG. 16 shows a TGA-MS characterization of a tosylate salt, form III.

FIG. 17 shows an XRD pattern of a tosylate salt, form IV.

FIG. 18 shows overlay of XRD patterns of tosylate salts, forms I through IV.

FIG. 19 shows an XRD pattern of an oxalate salt, form I.

FIG. 20 shows a DSC thermogram of an oxalate salt, form I.

FIG. 21 shows a TGA-MS characterization of an oxalate salt, form I.

FIG. 22 shows an XRD pattern of an oxalate salt, form II.

FIG. 23 shows a DSC thermogram of an oxalate salt, form II.

FIG. 24 shows a TGA-MS characterization of an oxalate salt, form II.

FIG. 25 shows an XRD pattern of oxalate salt, form III.

FIG. 26 shows a DSC thermogram of oxalate salt, form III.

FIG. 27 shows a TGA-MS characterization of oxalate salt, form III.

FIG. 28 shows an overlay XRD patterns of oxalate salts.

FIG. 29 shows an XRD pattern of fumarate salt, form I.

FIG. 30 shows an XRD pattern of fumarate salt, form II.

FIG. 31 shows an XRD pattern of fumarate salt, form III.

FIG. 32 shows a DSC thermogram of fumarate salt, form III.

FIG. 33 shows a TGA-MS characterization of fumarate salt, form III.

FIG. 34 shows an XRD pattern of fumarate salt, form IV.

FIG. 35 shows an XRD pattern of fumarate salt, form V.

FIG. 36 shows an overlay of XRD patterns of fumarate salts.

FIG. 37 shows an XRD pattern of camsylate salt, form I.

FIG. 38 shows an XRD pattern of camsylate salt, form II.

FIG. 39 shows a DSC thermogram of camsylate salt, form II.

FIG. 40 shows a TGA-MS characterization of camsylate salt, form II.

FIG. 41 shows an XRD pattern of camsylate salt, form III.

FIG. 42 shows a DSC thermogram of camsylate salt, form III.

FIG. 43 shows an overlay of XRD patterns of camsylate salts.

FIG. 44 shows an XRD pattern of citrate salt, form I.

FIG. 45 shows a DSC thermogram of citrate salt, form I.

FIG. 46 shows a TGA-MS characterization of citrate salt, form I.

FIG. 47 shows an XRD pattern of hydrochloride salt, form I.

FIG. 48 shows a DSC thermogram of hydrochloride salt, form I.

FIG. 49 shows a TGA-MS characterization of hydrochloride salt, form I.

FIG. 50 shows an XRD pattern of hydrochloride salt, form II.

FIG. 51 shows a DSC thermogram of hydrochloride salt, form II.

FIG. 52 shows a TGA-MS characterization of hydrochloride salt, form II.

FIG. 53 shows an XRD pattern of hydrochloride salt, form III.

FIG. 54 shows an XRD pattern of naphthalene-1,5-disulfonic acid, form I, wherein the bottom pattern is simulated based on single crystal data and the top line is an X-ray diffraction powder diffraction pattern.

FIG. 55 shows solubility data for free base and salt forms.

### DETAILED DESCRIPTION

Provided are polymorphic and salt forms of Compound A, which is also known as (1S,3S)—N¹—(5-(pentan-3-yl)pyrazolo[1,5-α]pyrimidin-7-yl)cyclopentane-1,3-diamine. Provided are polymorphic forms of the free base of Compound A along with polymorphic forms of salts of Compound A, including tosylate salts, oxalate salts, fumarate salts, camsylate salts, citrate salts, hydrochloride salts, and naphthalene-1,5-disulfonic acid salts. Such forms have been characterized by X-ray diffraction (XRD), differential scanning calorimetry (DSC), and thermogravimetric analysis coupled to mass spectrometry (TGA-MS). Such polymorphic and salt forms can have desirable solubility, dissolution, and pharmacokinetic properties.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

The singular forms “a”, “an”, and “the” include plural referents unless the context clearly dictates otherwise. Thus, for example, reference to “a droplet” includes a plurality of such droplets and reference to “the discrete entity” includes reference to one or more discrete entities, and so forth. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### DEFINITIONS

The terms “polymorphic form”, “polymorph form”, and “polymorph” are used interchangeably herein.

The term “AAC” refers to “accelerated aging conditions”.

The terms active agent, active pharmaceutical ingredient, pharmacologically active agent, and drug are used interchangeably herein to refer to a chemical material or compound which, when administered to an organism (human or animal) induces a desired pharmacologic and/or physiologic effect by local and/or systemic action.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease (e.g., reduction in viral titers).

A “therapeutically effective amount”, a “therapeutically effective dose” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of a compositions is an amount that is sufficient, when administered to the individual, to palliate, ameliorate, stabilize, reverse, prevent, slow or delay the progression of a disease state (e.g., cancer, etc.) present in the subject.

As used herein, the terms “determining,” “measuring,” “assessing,” and “assaying” are used interchangeably and include both quantitative and qualitative determinations.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant. Carriers are generally described herein and also in “Remington's Pharmaceutical Sciences” by E. W. Martin.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intrathecal, intramuscular, subcutaneous, and the like.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

### Salts and Polymorphic Forms

Provided are polymorphic forms of the free base of Compound A along with polymorphic forms of salts of Compound A. The free base of Compound A has the systematic IUPAC chemical name of (1S,3S)—N¹—(5-(pentan-3-yl)pyrazolo[1,5-α]pyrimidin-7-yl) cyclopentane-1,3-diamine and the chemical structure of formula (I):

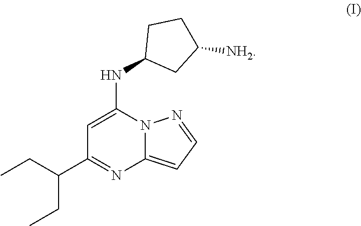 [SMILES: CCC(CC)c1cc(N[C@H]2CC[C@H](N)C2)n2nccc2n1]

In some cases the compound is a free base of Compound A. In other cases, the compound is a salt of Compound A, such as a salt selected from the group consisting of: tosylate, oxalate, fumarate, camsylate, citrate, hydrochloride, and naphthalene-1,5-disulfonate.

The free base or salt of Compound A can have a particular polymorphic form. In some cases such forms can be characterized by X-ray diffraction (XRD), such as powder XRD or single crystal XRD. XRD can be used to determine the crystal structure of the polymorphic form, or to determine that the form is amorphous. Such forms can also be characterized by differential scanning calorimetry (DSC) and thermogravimetric analysis coupled to mass spectrometry (TGA-MS). The results of DSC, which are sometimes referred to as a thermogram, can show how much heat is needed to increase the temperature of a sample (e.g. the polymorphic form) compared to a reference material. With TGA-MS, the sample is heated and changes in mass are measured as a function of time and temperature (i.e. the thermogravimetric analysis). Furthermore, compounds liberated during the heating are subjected to mass spectrometry.

In some cases, the polymorphic forms of the free base or salt of Compound A has an XRD, DSC, or TGA-MS spectrum as substantially as shown in the figures. As used herein, the term “substantially as shown in” when referring to an XRD, DSC, and TGA-MS means that a pattern or profile that is not necessarily identical to those depicted herein, but that falls within the limits of experimental error or deviations, when considered by one of ordinary skill in the art, would be encompassed.

In relation to XRD, experimental error and deviations include the 2θ value of a peak in the XRD spectrum. For instance, a given peak of a test sample can be located ±0.2 degrees 2θ of the peak in the test sample, such as ±0.1 degrees 2θ. In some cases, the XRD pattern of a test sample has peaks that correspond to the most intense peaks in the reference spectrum. For example, the XRD pattern of the test sample can have four peaks that are located ±0.2 degrees 2θ of the four most intense peaks of the reference XRD pattern, or six peaks located ±0.2 degrees 2θ of the six most intense peaks.

In relation to DSC, experimental error and deviations include the temperature of a peak in the DSC thermogram. For instance, a given peak of a test sample can be located within 20° C. of the reference peak, such as within 10° C. or within 5° C. In some cases, the DSC thermogram of the test sample has one or more peaks corresponding to the one or more most intensity peaks in the reference thermogram. In some cases, the DSC thermogram has a single peak. It is understood that the “peaks” in a DSC thermogram are commonly negative peaks, e.g. the intensity decreases instead of increasing.

In relation to TGA-MS, experimental error and deviations include the include the temperature of a peak in the thermogravimetric spectrum. In some cases, the thermogravimetric spectrum of the test sample has one or more peaks corresponding to the one or more most intensity peak in the reference thermogram such as two or more peaks corresponding to the two or more most intense peaks in the reference spectrum, or three or more peaks corresponding to the three or more most intense peaks in the reference spectrum. Experimental error and deviations also include the detection of a particular compound, as measured by its m/z ratio, in the mass spectrum of the TGA-MS analysis.

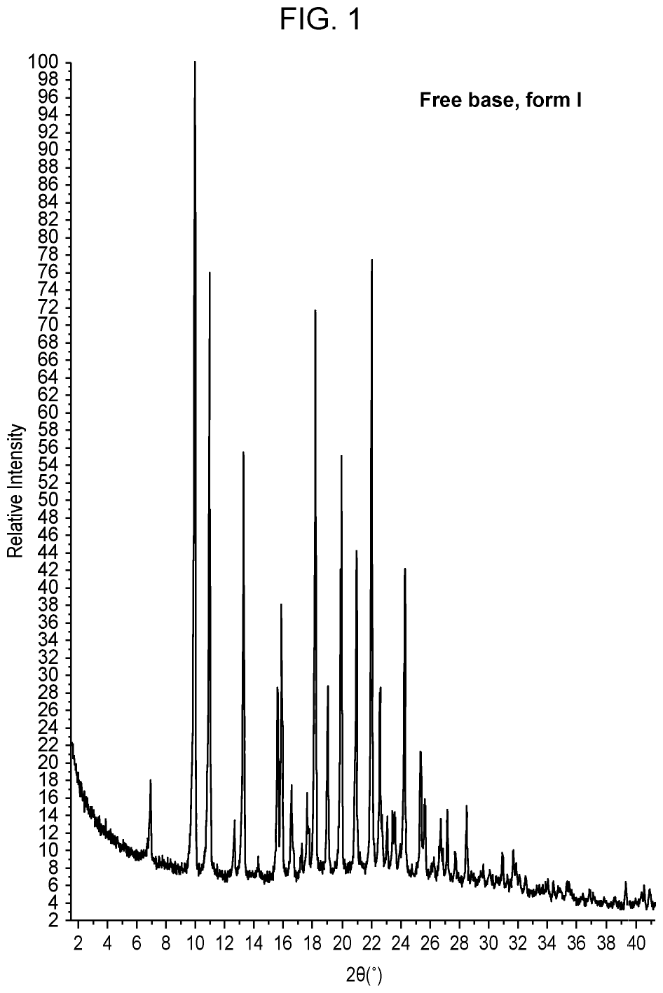

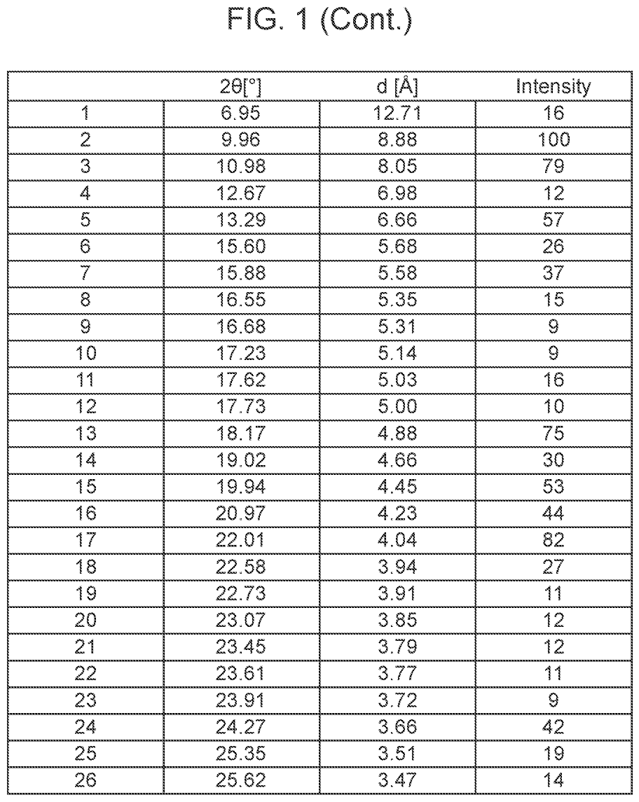

Provided are polymorphic forms I, II, and III of the free base of Compound A. As used herein, “form I” is used interchangeably with “Form I” and “form II” is used interchangeably with “Form II”. The forms can have XRD, DSC, and TGA-MS characterizations substantially as shown in FIGS. 1-6, 7A-7C, 8A-8C, and 9A-9C. The forms can of the free base can be characterized by XRD peaks with 2θ values, ±0.2 degrees 2θ, at:

Form I: 10.0, 11.0, 13.3, 18.2, and 22.0

Form II: 7.2, 14.4, 21.7, and 22.5

Form III: amorphous

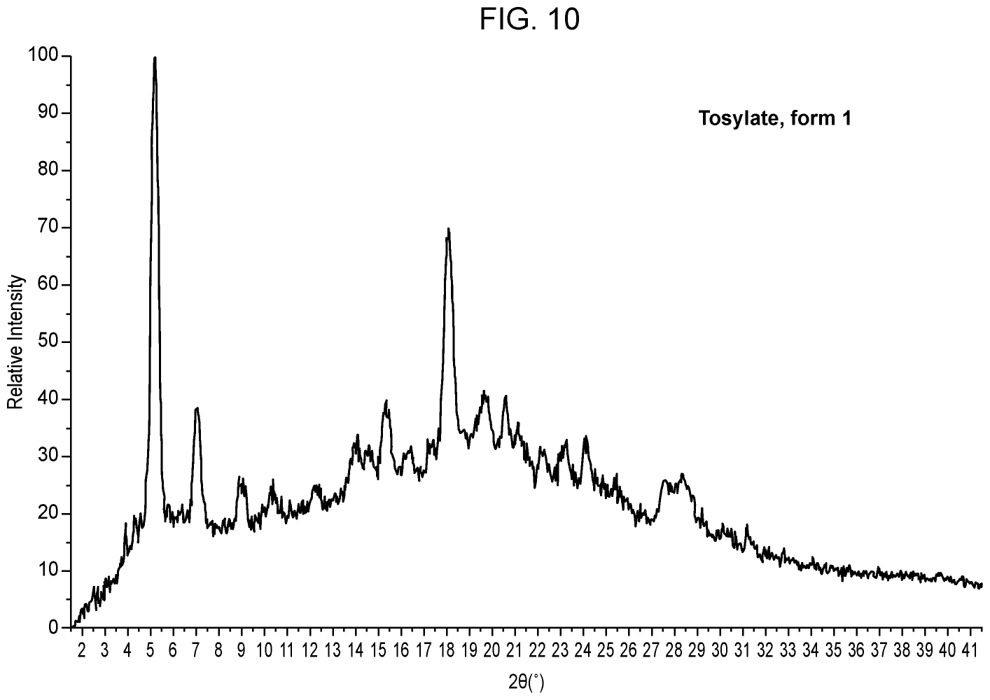

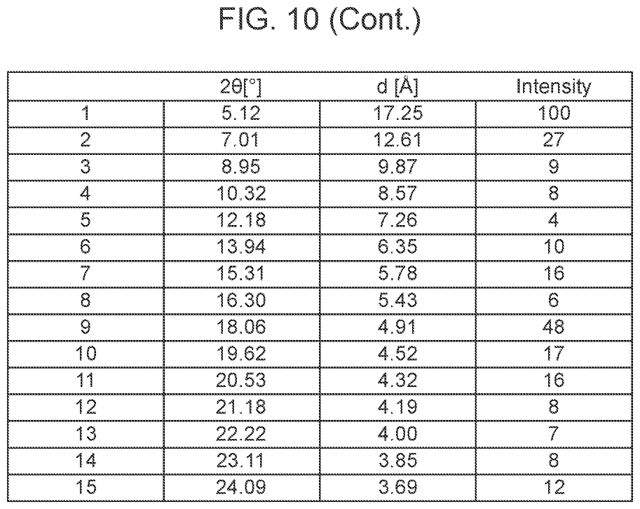

Provided are polymorphic forms I, II, III, and IV of a tosylate salt of Compound A. Such forms can be characterized by XRD, DSC, and TGA-MS spectra as shown in FIGS. 10-17. In some cases the polymorphic forms of the tosylate salt have XRD peaks, ±0.2 degrees 2θ, of:

Form I: 5.1, 7.0, and 18.1

Form II: 5.2, 7.5, 18.1, 19.1, 20.6

Form III: 6.5, 15.4, 18.4, 21.8

Form IV: 5.4, 6.2, 15.7, 18.2

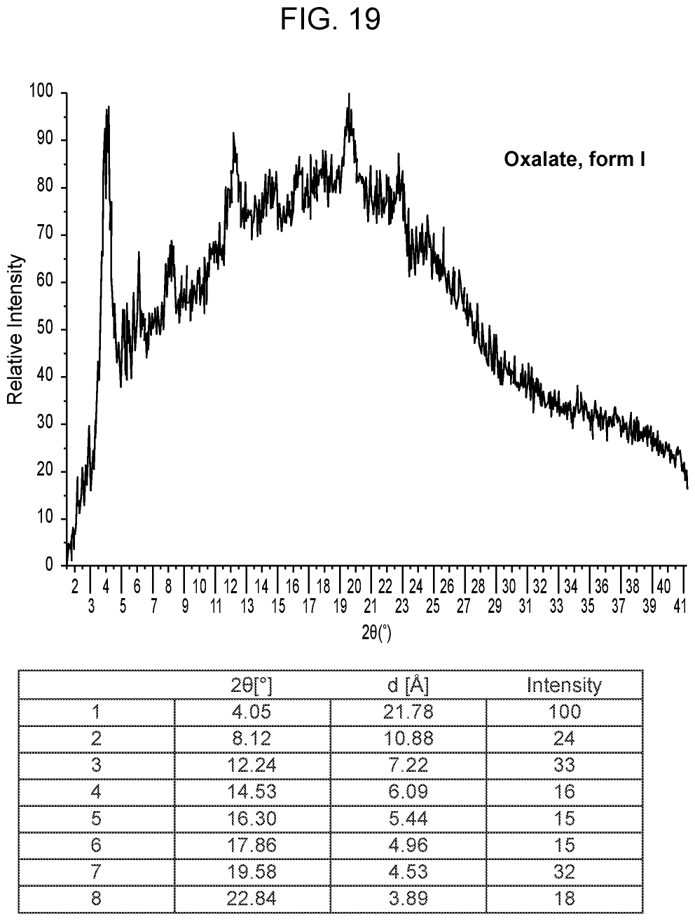

Provided are polymorphic forms I, II, and III of an oxalate salt of Compound A. Such forms can be characterized by XRD, DSC, and TGA-MS spectra as shown in FIGS. 19-27. In some cases the polymorphic forms of the oxalate salt have XRD peaks, ±0.2 degrees 2θ, of:

Form I: 4.1, 8.1, 12.2, and 19.6

Form II: 6.1, 8.2, 12.3, 19.6, 22.8

Form III: 10.8, 18.3, 19.9

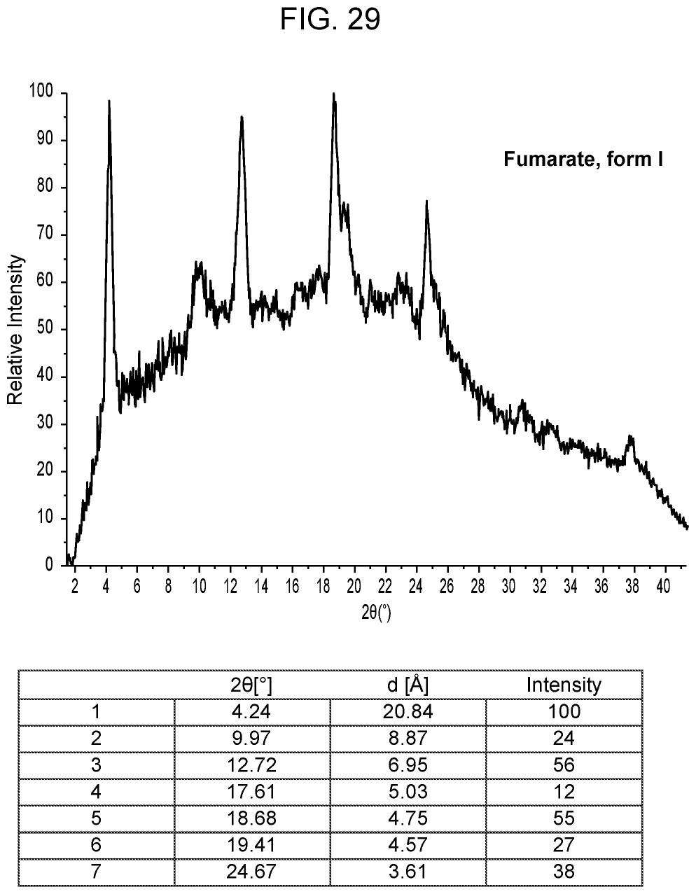

Provided are polymorphic forms I, II, III, IV, and V of a fumarate salt of Compound A. Such forms can be characterized by XRD, DSC, and TGA-MS spectra as shown in FIGS. 29-35. In some cases the polymorphic forms of the fumarate salt have XRD peaks, ±0.2 degrees 2θ, of:

Form I: 4.2, 12.7, 18.7, 24.7

Form II: 4.3, 8.0, 11.5, 11.9, 24.5

Form III: 10.6, 11.5, 21.0 22.2, 24.6

Form IV: 3.9, 13.3, 23.3, 24.3

Form V: 4.1, 12.4, 20.5, 21.0

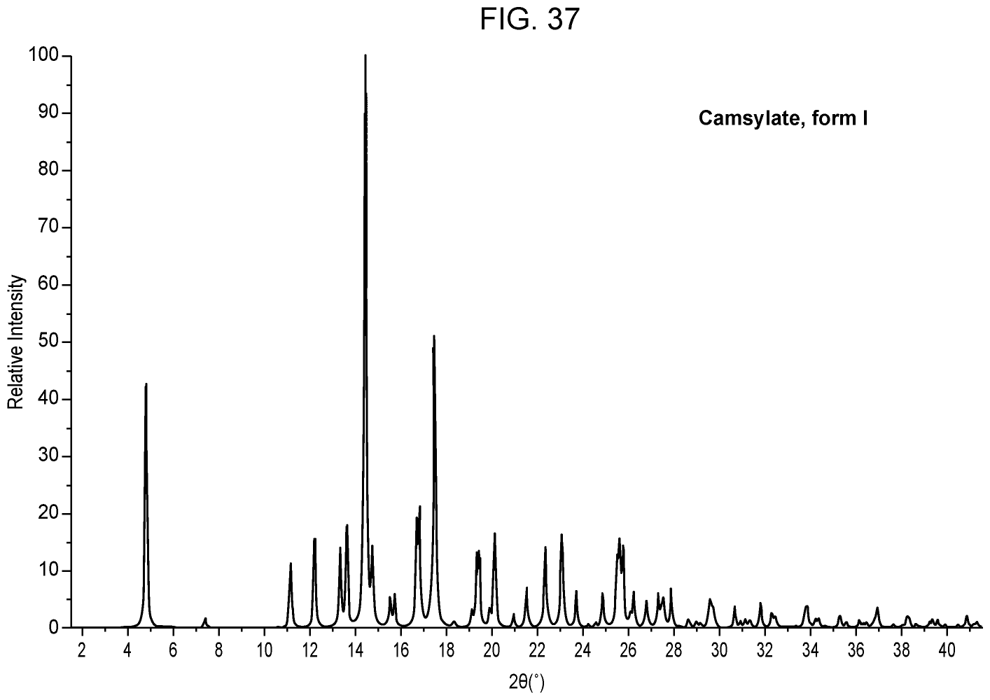

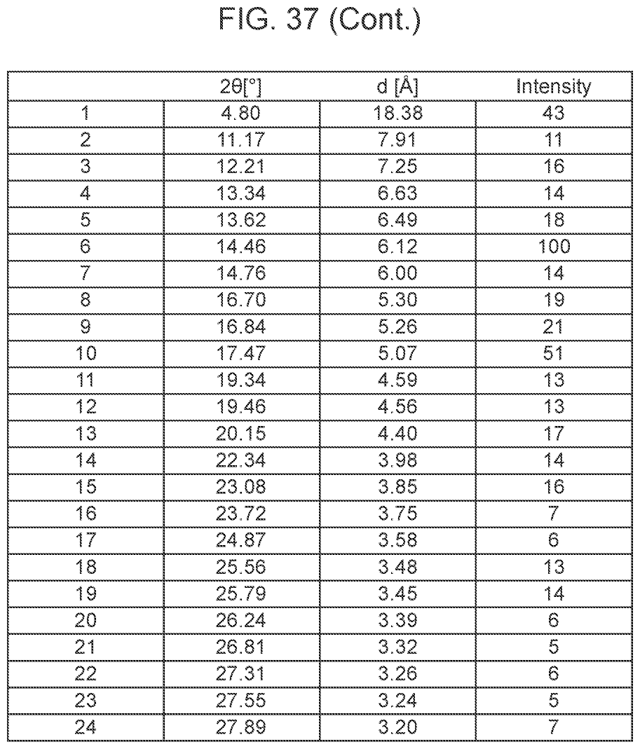

Provided are polymorphic forms I, II, and III of a camsylate salt of Compound A. The term “cas” is an abbreviation for camsylate. Such forms can be characterized by XRD, DSC, and TGA-MS spectra as shown in FIGS. 37-42. In some cases the polymorphic forms of the camsylate salt have XRD peaks, ±0.2 degrees 2θ, of:

Form I: 4.8, 14.5, 16.8, 17.5, 20.2

Form II: 5.3, 12.6, 15.2, 17.2

Form III: 5.0, 12.2, 14.7, 23.0

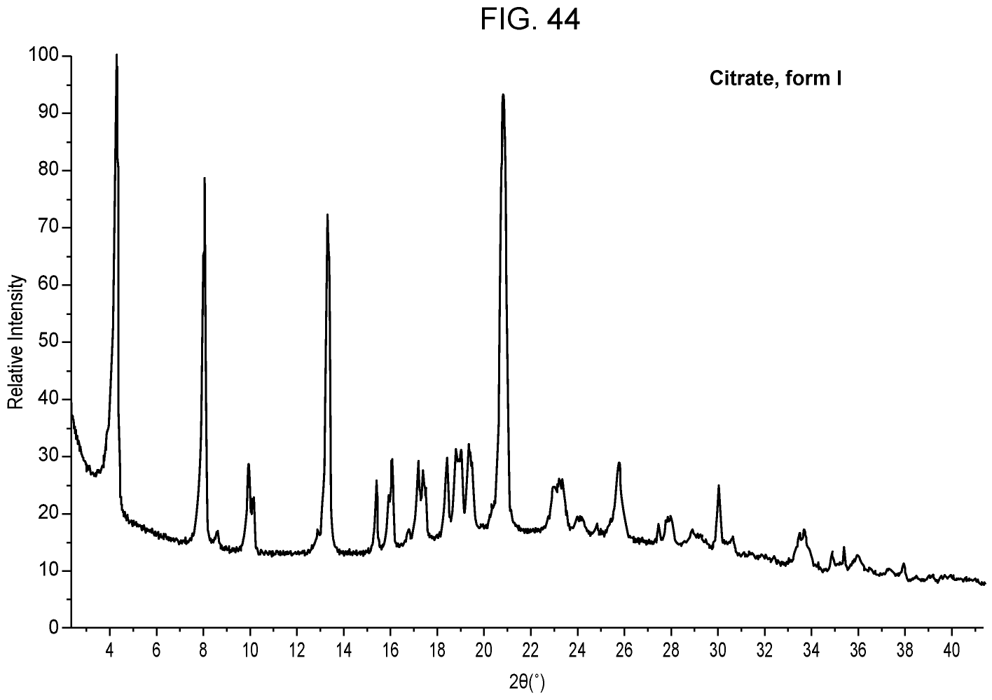

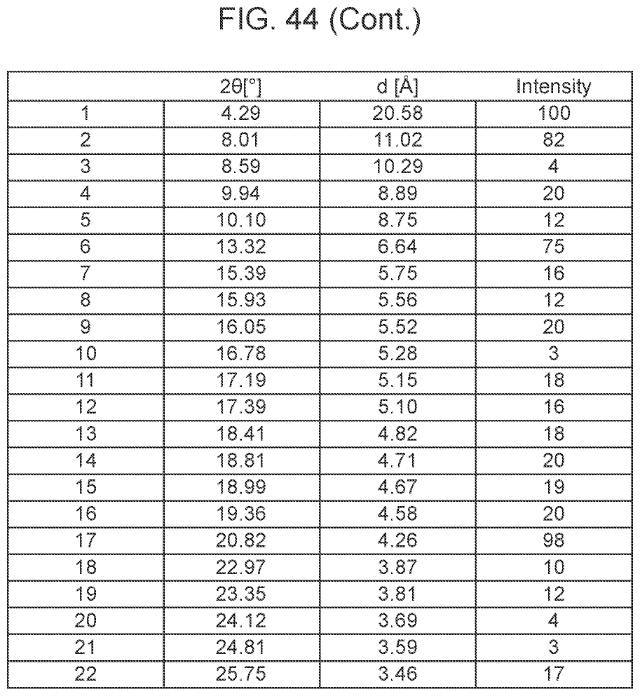

Provided is polymorphic form I of a citrate salt of Compound A. Such forms can be characterized by XRD, DSC, and TGA-MS spectra as shown in FIGS. 44-46. In some cases the polymorphic form of the citrate salt have XRD peaks, ±0.2 degrees 2θ, of:

Form I: 4.3, 8.0, 13.3, 20.8

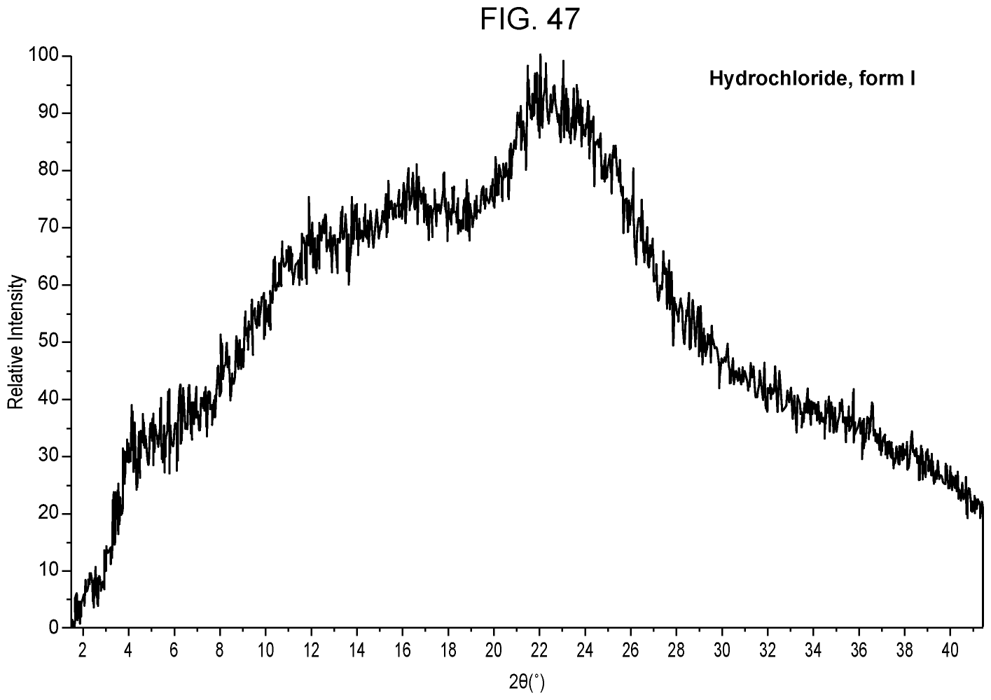

Provided are polymorphic forms I, II, and III of a hydrochloride salt of Compound A. Such forms can be characterized by XRD, DSC, and TGA-MS spectra as shown in FIGS. 47-53. In some cases the polymorphic form of the hydrochloride salt have XRD peaks, ±0.2 degrees 2θ, of:

Form I: amorphous X-ray diffraction pattern

Form II: 9.9, 17.7, 21.3, 24.8

Form III: 9.4, 20.8, 22.7, 25.6

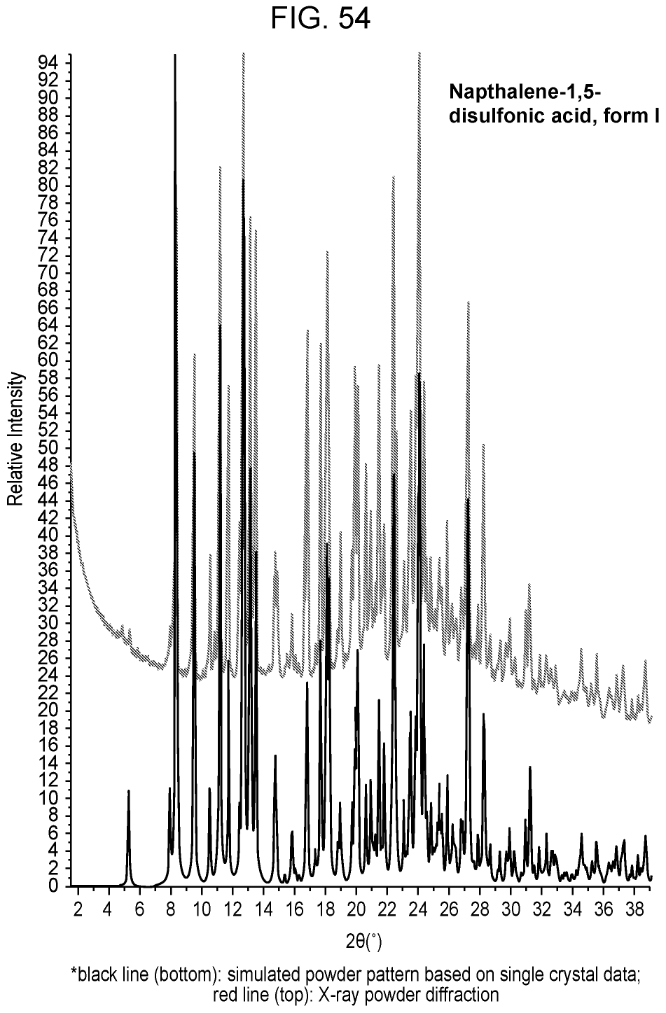

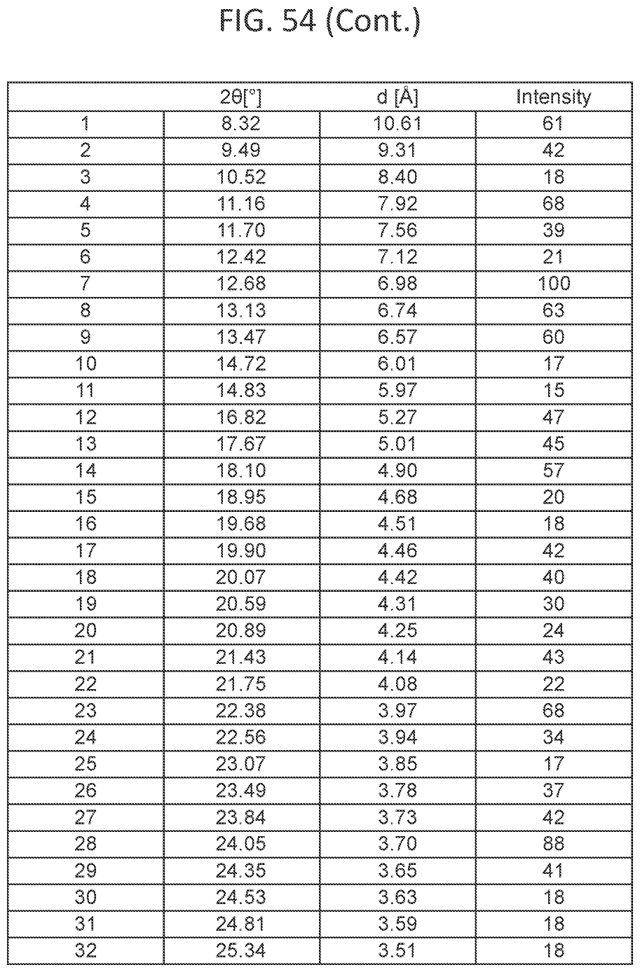

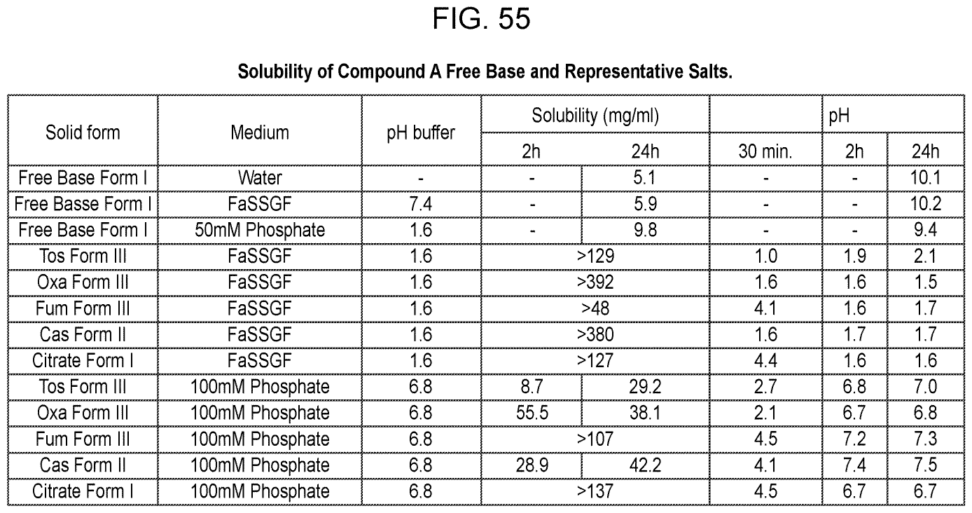

Provided is polymorphic form I of a naphthalene-1,5-disulfonic acid salt of Compound A. Such forms can be characterized by XRD, DSC, and TGA-MS spectra as shown in FIG. 54. In some cases the polymorphic form of the naphthalene-1,5-disulfonic acid salt have XRD peaks, ±0.2 degrees 2θ, of:

Form I: 8.3, 12.7, 13.1, 22.4, 24.1

In some cases, the compound is a mono-salt, e.g. the compound includes the structure of formula (I) and one equivalent of HCl. In other cases, the compound is a di-salt, e.g. the compound includes the structure of formula (I) and two equivalents of HCl. The compound can also be a mono-salt or di-salt of tosylate, oxalate, fumarate, camsylate, citrate, or naphthalene-1,5-disulfonate. The compound can also have a non-integer number of equivalents, e.g. 1.5 HCl per formula (I) molecule or 2.5 HCl per formula (I) molecule.

### Methods of Preparing Polymorph Forms

Also provided are methods of synthesizing a polymorph of a tosylate salt, an oxalate salt, a fumarate salt, a camsylate salt, a citrate salt, a hydrochloride salt, or a naphthalene-1,5-disulfonic acid salt of formula (I).

In some cases, the method includes: (i) contacting a free base of formula (I) with a solvent and a compound comprising a tosylate moiety, an oxalate moiety, a fumarate moiety, a camsylate moiety, a citrate moiety, a chloride moiety, or a naphthalene-1,5-disulfonic acid moiety, thereby generating a composition comprising the solvent and a salt of formula (I); and (ii) evaporating the solvent to generate a solid product.

In some cases, the compound comprising the salt moiety is the corresponding acid. For instance, in some cases the compound is oxalic acid, wherein deprotonating oxalic acid twice results in the oxalate anion. Thus, contacting the free base of Compound A with oxalic acid can result in a protonated and cationic analog of formula (I) along with an anionic oxalate ion. As another example, the compound can be naphthalene-1,5-disulfonic acid (also known as Armstrong's acid), which can result in the protonation of the formula (I) structure and the formation of the anionic naphthalene-1,5-disulfonate anion. Similarly, the compound can be camphorsulfonic acid to form the camsylate salt, fumaric acid for the fumarate salt, or citric acid for the citrate salt.

Provided are methods of synthesizing a polymorph of a free base of formula (I), e.g. form I, form II, or form III. In some cases, the method is a method of synthesizing form III, e.g. by spray drying a solution of another form of the free base, such as form I. For instance, the method can include spray drying a methanol solution of form I, e.g. in the presence of a particle such as PVP K30.

### Pharmaceutical Compositions

Provided are pharmaceutical compositions comprising a polymorph as described herein and a pharmaceutically acceptable carrier. In some cases the pharmaceutical composition further comprises a pharmaceutically acceptable excipient, a pharmaceutically acceptable diluent, a pharmaceutically acceptable adjuvant, a pharmaceutically acceptable disintegrant, a pharmaceutically acceptable lubricant, or a combination thereof. In some cases the pharmaceutical composition is an aqueous solution. In other embodiments the pharmaceutical composition is a tablet or pill.

The pharmaceutical compositions can be formulated to contain suitable pharmaceutically acceptable carriers, and optionally can comprise excipients and auxiliaries that facilitate processing of the polymorphic forms described herein into preparations that can be used pharmaceutically. The mode of administration generally determines the nature of the carrier. For example, formulations for parenteral administration can include aqueous solutions of the active compounds in water-soluble form. Carriers suitable for parenteral administration can be selected from among saline, buffered saline, dextrose, water, and other physiologically compatible solutions. Exemplary carriers for parenteral administration are physiologically compatible buffers such as Hanks's solution, Ringer's solution, or physiologically buffered saline. For tissue or cellular administration, penetrants appropriate to the particular barrier to be permeated are used in the formulation. Such penetrants are generally known in the art. For preparations including proteins, the formulation can include stabilizing materials, such as polyols (e.g., sucrose) and/or surfactants (e.g., nonionic surfactants), and the like.

Alternatively, formulations for parenteral use can include dispersions or suspensions of polymorphic forms described herein prepared as appropriate oily injection suspensions. Suitable lipophilic solvents or vehicles include fatty oils, such as sesame oil, and synthetic fatty acid esters, such as ethyl oleate or triglycerides, or liposomes. Aqueous injection suspensions can contain substances that increase the viscosity of the suspension, such as sodium carboxymethylcellulose, sorbitol, dextran, and mixtures thereof. Optionally, the suspension also can contain suitable stabilizers or agents that increase the solubility of the compounds to allow for the preparation of highly concentrated solutions. Aqueous polymers that provide pH-sensitive solubilization and/or sustained release of the active agent also can be used as coatings or matrix structures, e.g., methacrylic polymers, such as the EUDRAGIT™ series available from Rohm America Inc. (Piscataway, N.J.). Emulsions, e.g., oil-in-water and water-in-oil dispersions, also can be used, optionally stabilized by an emulsifying agent or dispersant (surface active materials; surfactants). Suspensions can contain suspending agents such as ethoxylated isostearyl alcohols, polyoxyethlyene sorbitol and sorbitan esters, microcrystalline cellulose, aluminum metahydroxide, bentonite, agar-agar, gum tragacanth, and mixtures thereof.

Liposomes containing the polymorphic forms described herein also can be employed for parenteral administration. Liposomes generally are derived from phospholipids or other lipid substances. The compositions in liposome form also can contain other ingredients, such as stabilizers, preservatives, excipients, and the like. Exemplary lipids include phospholipids and phosphatidyl cholines (lecithins), both natural and synthetic. Methods of forming liposomes are known in the art. See, e.g., Prescott (Ed.), Methods in Cell Biology, Vol. XIV, p. 33, Academic Press, New York (1976).

In some embodiments, the polymorph, or composition thereof, disclosed herein is formulated for oral administration using pharmaceutically acceptable carriers well known in the art. Preparations formulated for oral administration can be in the form of tablets, pills, capsules, cachets, dragees, lozenges, liquids, gels, syrups, slurries, elixirs, suspensions, or powders. To illustrate, pharmaceutical preparations for oral use can be obtained by combining the active compounds with a solid excipient, optionally grinding the resulting mixture, and processing the mixture of granules, after adding suitable auxiliaries if desired, to obtain tablets or dragee cores. Oral formulations can employ liquid carriers similar in type to those described for parenteral use, e.g., buffered aqueous solutions, suspensions, and the like.

Exemplary oral formulations include tablets, dragees, and gelatin capsules. These preparations can contain one or more excipients, which include, without limitation: a) diluents, such as microcrystalline cellulose and sugars, including lactose, dextrose, sucrose, mannitol, or sorbitol; b) binders, such as sodium starch glycolate, croscarmellose sodium, magnesium aluminum silicate, starch from com, wheat, rice, potato, etc.; c) cellulose materials, such as methylcellulose, hydroxypropylmethyl cellulose, and sodium carboxymethylcellulose, polyvinylpyrrolidone, gums, such as gum arabic and gum tragacanth, and proteins, such as gelatin and collagen; d) disintegrating or solubilizing agents such as cross-linked polyvinyl pyrrolidone, starches, agar, alginic acid or a salt thereof, such as sodium alginate, or effervescent compositions; e) lubricants, such as silica, talc, stearic acid or its magnesium or calcium salt, and polyethylene glycol; f) flavorants and sweeteners; g) colorants or pigments, e.g., to identify the product or to characterize the quantity (dosage) of active compound; and h) other ingredients, such as preservatives, stabilizers, swelling agents, emulsifying agents, solution promoters, salts for regulating osmotic pressure, and buffers.

Examples of carriers include, but are not limited to, aluminum monostearate, aluminum stearate, carboxymethylcellulose, carboxymethylcellulose sodium, crospovidone, glyceryl isostearate, glyceryl monostearate, hydroxyethylcellulose, hydroxymethylcellulose, hydroxyoctacosanyl hydroxystearate, hydroxypropylcellulose, hydroxypropylmethylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, and silicone emulsion. It should be understood, however, that the carriers selected for the pharmaceutical compositions provided in the present disclosure, and the amounts of such carriers in the composition, may vary depending on the method of formulation (e.g., dry granulation formulation, solid dispersion formulation).

In certain variations, the pharmaceutical composition comprises a polymorph as described herein and at least one pharmaceutically acceptable carrier selected from the group consisting of hydroxypropylmethylcellulose, mannitol, crospovidone, poloxamer, colloidal silicon dioxide, microcrystalline cellulose, magnesium stearate, and any mixtures thereof. In another variation, the pharmaceutical composition comprises a polymorph as described herein, hydroxypropylmethylcellulose, and at least one additionally pharmaceutically acceptable carrier selected from the group consisting of mannitol, crospovidone, poloxamer, colloidal silicon dioxide, microcrystalline cellulose, magnesium stearate, and any mixtures thereof.

Regardless of the route of administration selected, the agents/compounds of the present disclosure are formulated into pharmaceutically-acceptable dosage forms by conventional methods known to those of skill in the art using pharmaceutically acceptable carriers well-known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly (orthoesters), and poly (anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition of the disclosure is “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutical compositions of the disclosure may, optionally, contain additional ingredients and/or materials commonly used in such pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Pharmaceutical compositions which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable carrier. The ointments, pastes, creams and gels may contain excipients. Powders and sprays may contain excipients and propellants.

Pharmaceutical compositions suitable for parenteral administrations comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, to prolong the effect of a drug (e.g., pharmaceutical formulation), it is desirable to slow its absorption from subcutaneous or intramuscular injection. This may be accomplished by use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

### Methods of Use

Provided are methods of treating a condition in a subject by administering to the subject a polymorph as described herein or a pharmaceutical composition as described herein including such a polymorph. In some cases, the condition is cancer or an autoimmune disease. In some embodiments the subject is a human, e.g. a human with cancer or an autoimmune disease. In some cases the method is performed with a kit as described herein.

A pharmaceutical composition of the present disclosure may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a pharmaceutical composition of the present disclosure may be administered in conjunction with other treatments. A pharmaceutical composition of the present disclosure maybe encapsulated or otherwise protected against gastric or other secretions, if desired.

A suitable, non-limiting example of a dosage of the compounds according to the present disclosure is from about 1 ng/kg to about 1000 mg/kg, such as from about 1 mg/kg to about 100 mg/kg, including from about 5 mg/kg to about 50 mg/kg. Other representative dosages of a PI3K inhibitor include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg.

### Kits

Provided are kits that include a polymorph as described herein, or a pharmaceutical composition comprising such a polymorph, and instructions for using the polymorph or pharmaceutical composition. In some cases, the instructions direct a user to administer the polymorph or pharmaceutical composition to a subject having a condition, e.g. cancer or an autoimmune disorder. In some cases the instructions direct administration by oral administration, injection, or any other suitable route of administration. In some cases the subject is human. The instructions can direct administration based on any feature described in the methods of use section above. The instructions can have any suitable format, e.g. printed on a label or contained in an electronic format on an electronic storage media such as a universal serial (USB) storage device.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Celsius, and pressure is at or near atmospheric. Standard abbreviations may be used, e.g., s or sec, second(s); min, minute(s); h or hr, hour(s); and the like.

### Experimental Methodology

The horizontal axis of DSC (differential scanning calorimetry) thermograms in the figures is 25 to 300° C. unless otherwise specified. For TGA-MS (thermal gravimetric mass spectrometry) in the figures, the graph starting higher is the sample weight, and the graph starting lower is the heat flow.

### TGA/SDTA and TGA-MS Analysis

Mass loss due to solvent or water loss from the crystals was determined by TGA/DSC. Monitoring the sample weight, during heating in a TGA/DSC 3+ STARe system (Mettler-Toledo GmbH, Switzerland), resulted in a weight vs. temperature curve and a heat flow signal. The TGA/DSC 3+ was calibrated for temperature with samples of indium and aluminum. Samples (circa 1 mg) were weighed in 100 μL aluminum crucibles and sealed. The lids were pin-holed, and the crucibles heated in the TGA from 25 to 300° C. at a heating rate of 10° C./min. Dry N2 gas was used for purging.

The gases coming from the TGA samples were analyzed by a mass spectrometer Omnistar GSD 301 T2 (Pfeiffer Vacuum GmbH, Germany). The latter is a quadrupole mass spectrometer, which analyzes masses in the m/z range of 0-200 amu.

### DSC Analysis

Thermal events were obtained from DSC thermograms, which were recorded with a heat flux DSC3+ STARe system (Mettler-Toledo GmbH, Switzerland). The DSC3+ was calibrated for temperature and enthalpy with indium (m.p.=156.6° C.; δHf=28.45 J/g) and zinc (m.p.=419.6° C.; δHf=107.5 J/g). Samples (circa 1 mg) were sealed in standard 40 μL aluminum pans, pin-holed and heated in the DSC from 25° C. to 300° C., at a heating rate of 10° C./min if not specified differently. Dry N2 gas, at a flow rate of 50 mL/min was used to purge the DSC equipment during measurement.

### HPLC Analysis

Details on the HPLC analyses for the assay and purity determination are provided in separate documents. The HPLC method summary is provided in the tables below.

| Parameter | Conditions |
|---|---|
| Column | Phenomenex Luna Phenyl-Hexyl |
| | 100A 150 mm × 4.6 mm, 5 μm |
| Column temperature | 25° C. ± 3° C. |
| Sample temperature | 5° C. ± 5° C. |
| Flow rate | 1.0 ml/min |
| Needle wash | MeOH (extended) |
| Injection volume | 10 μL |
| Detection wavelength | 220 nm |
| Mobile phase A | 20 mM ammonium acetate in water |
| Mobile phase B | Acetonitrile |

| Gradient | | |
|---|---|---|
| Time [min] | A [%] | B [%] |
| 0 | 95 | 5 |
| 2.0 | 95 | 5 |
| 11.0 | 10 | 90 |
| 19.0 | 10 | 90 |
| 20.0 | 95 | 5 |
| 25.0 | 95 | 5 |

### pH Determination

The pH was recorded with a Metrohm 913 PH meter, equipped with a WTW SenTix® MIC-B electrode.

### Dynamic Vapor Sorption

Moisture sorption isotherms were collected on a DVS-1 system from Surface Measurement Systems (London, UK). A sample size of about 8 mg of solid material was used. The material was subjected to a relative humidity profile of 40-95-0-40% RH with an increase of 10% RH level per step. Weight equilibration time was set for a minimum holding time of 60 minutes and a maximum of 6 hours per relative humidity step, and with a dm/dt 0.002%/min.

### Example 1

### Synthesis of Free Base of Compound A

The precursor to Compound A was prepared according to the procedure of US Patent Application Publication 2020/0131189, which is incorporated herein by reference.

Generating form I: HCl and the precursor were added to a reactor and stirred at 20° C. to 25° C. until the reaction as monitored by HPLC showed less than 0.5% of the precursor remaining. The pH of the reaction mixture is adjusted to 6 to 7 with 48% aqueous NaOH, and methyl tert butyl ether (MTBE) is added. The pH is then further adjusted to pH=14 with 48% aqueous NaOH, and the layers are separated. The aqueous layer is back extracted with MTBE, and the combined organic layers are washed with water. The MTBE solution is then concentrated partially under atmospheric pressure to remove residual water and then cooled to 20° C. to 25° C. The solution is polish filtered and the filtrate concentrated and co evaporated with n-heptane. The residue is then dissolved in n-heptane at 95° C. to 100° C. and then slowly cooled to 20° C. to 25° C. to crystallize the product. The material is collected by filtration, washed with n-heptane, and then dried to afford Compound I Free Base Form 1. The form was characterized by XRD, DSC, and TGA-MS as shown in FIGS. 1-3.

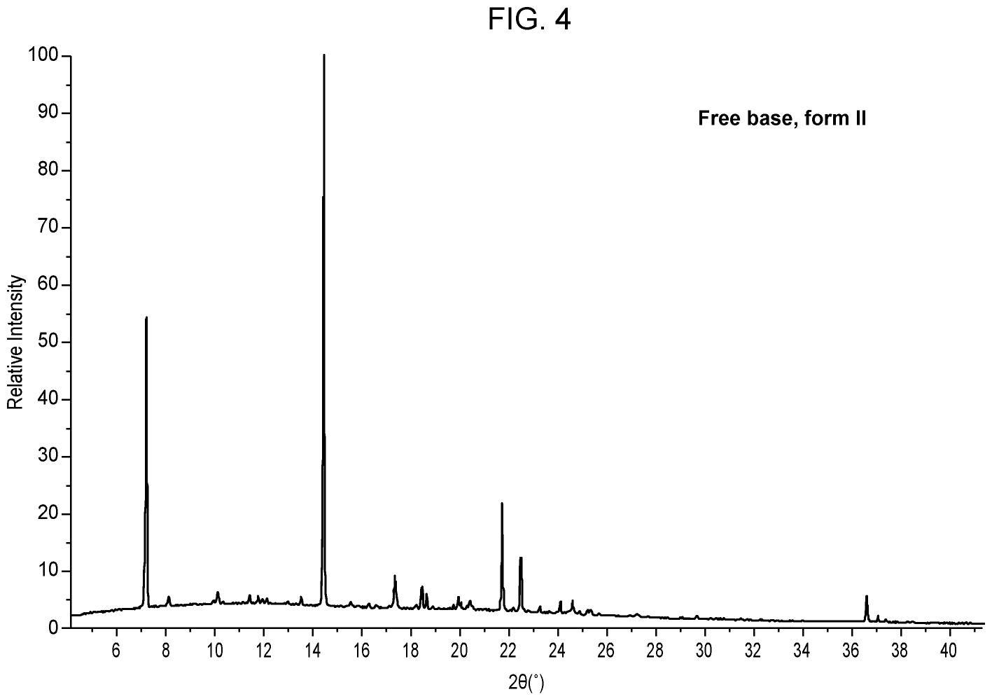

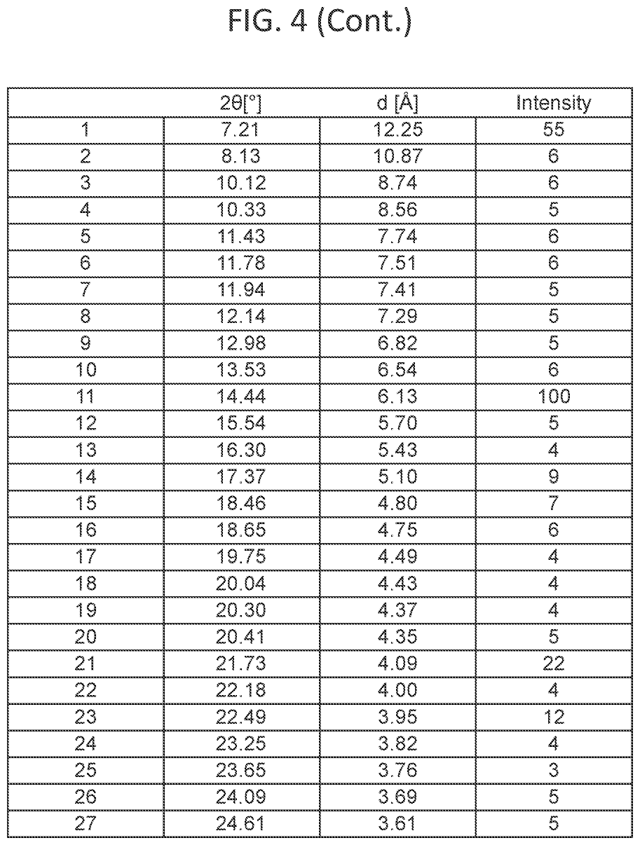

Generating form II: Form I of the free base was dissolved in a solvent, frozen in liquid nitrogen, and then freeze dried with vacuum (Alpha 2-4 LD, Martin Christ) for 18 hours. Alternatively, form I was dissolved in a solvent and the solvent was allowed to slowly evaporate. As shown in the table below, such methods resulted in the regeneration of form I, the generation of form II, generation of mixtures of forms I and II, or an amorphous product. Form II was characterized by XRD, DSC, and TGA-MS as shown in FIGS. 4-6. XRPD refers to X-ray powder diffraction.

| | Concentration | Crystallization | |
|---|---|---|---|
| Solvent (V/V) | (mg/ml) | method | XRPD outcome |
| EtOH/water (9/1) | 40.0 | Freeze-drying | Form I |
| EtOH/water (9/1) | 40.0 | Freeze-drying | Form II + Form I |
| EtOH | 41.0 | Slow evaporation | Amorphous |
| EtOH | 40.0 | Slow evaporation | Form II + Form I |
| EtOH | 40.0 | Fast evaporation | Form II + Form I |
| EtOH/water (7/3) | 20.0 | Freeze-drying | Form II |
| EtOH/water (7/3) | 42.1 | Freeze-drying | Form II |
| EtOH/water (7/3) | 42.1 | Freeze-drying | Form II |
| EtOH/water (7/3) | 42.1 | Freeze-drying | Form II |
| EtOH/water (7/3) | 42.1 | Freeze-drying | Form II |
| EtOH/water (7/3) | 42.1 | Freeze-drying | Form II |
| EtOH/water (7/3) | 42.1 | Freeze-drying | Form II |
| EtOH/water (7/3) | 42.1 | Freeze-drying | Form II |
| EtOH/water (7/3) | 42.1 | Freeze-drying | Form II |
| EtOH/water (7/3) | 39.8 | Freeze-drying | Form II |
| EtOH/water (7/3) | 39.8 | Freeze-drying | Form II |
| EtOH/water (7/3) | 39.8 | Freeze-drying | Form II |
| EtOH/water (7/3) | 39.8 | Freeze-drying | Form II |
| EtOH/water (7/3) | 39.8 | Freeze-drying | Form II |
| EtOH/water (7/3) | 39.8 | Freeze-drying | Form II |

### Generating Form III

Initial attempts to generate an amorphous form of the free base were conducted by slow evaporation of a form I solution. In particular, form I was dissolved in ethanol according to the chart below and then allowed to slowly evaporate.

| Vial Size | Form I | Concentration |
|---|---|---|
| (ml) | (mg) | (mg/ml) |
| 1.8 | 30 | 41 |
| 1.8 | 30 | 60 |
| 8 | 30 | 41 |
| 8 | 30 | 20 |
| 40 | 30 | 41 |

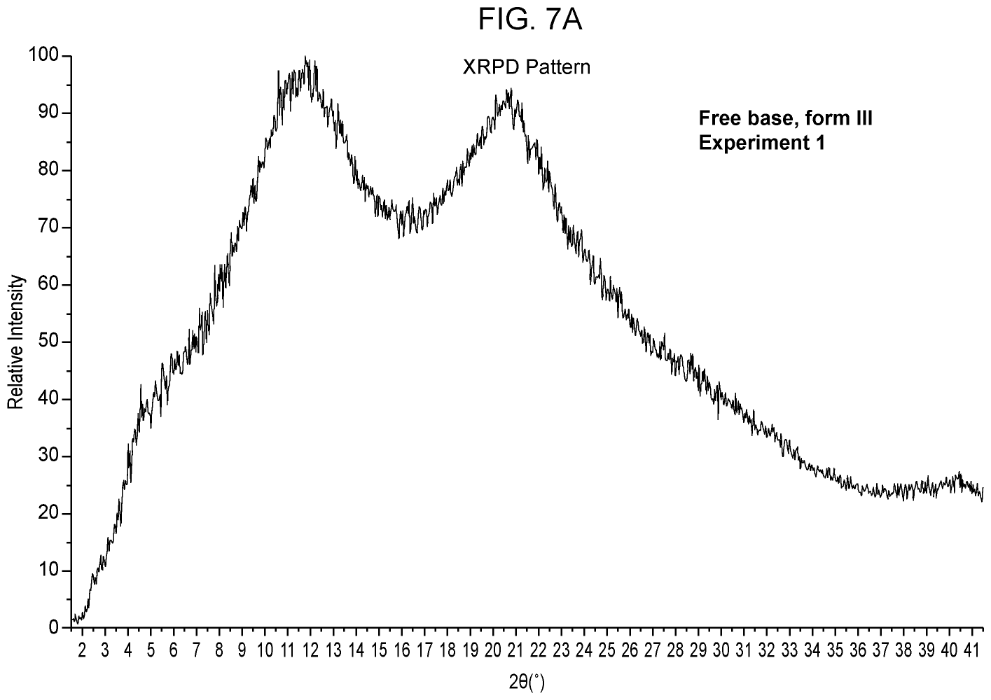

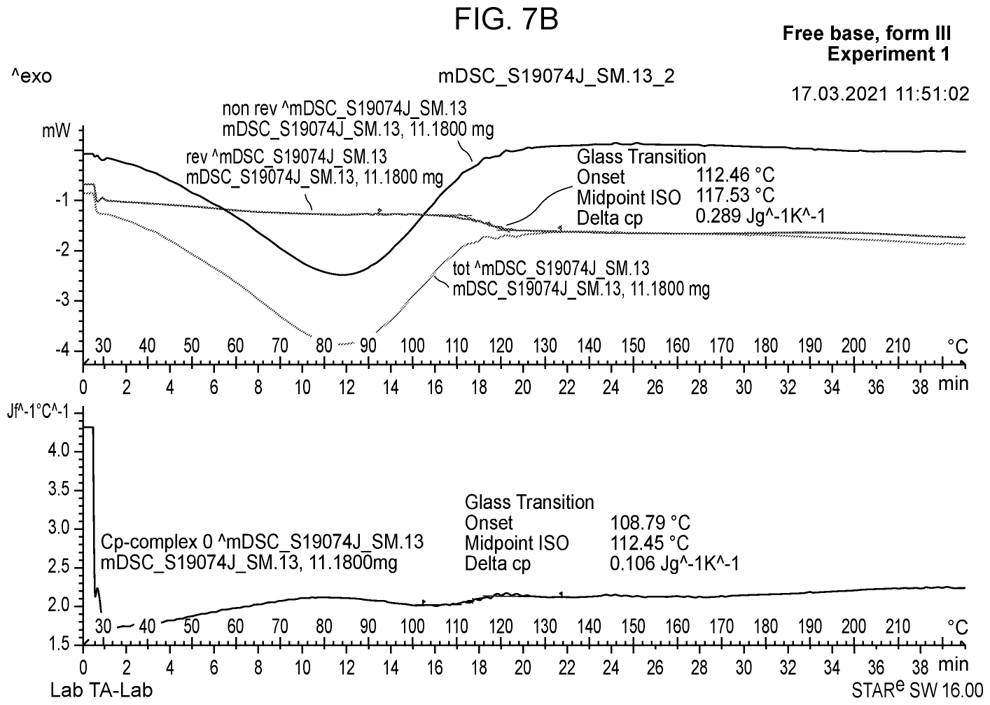

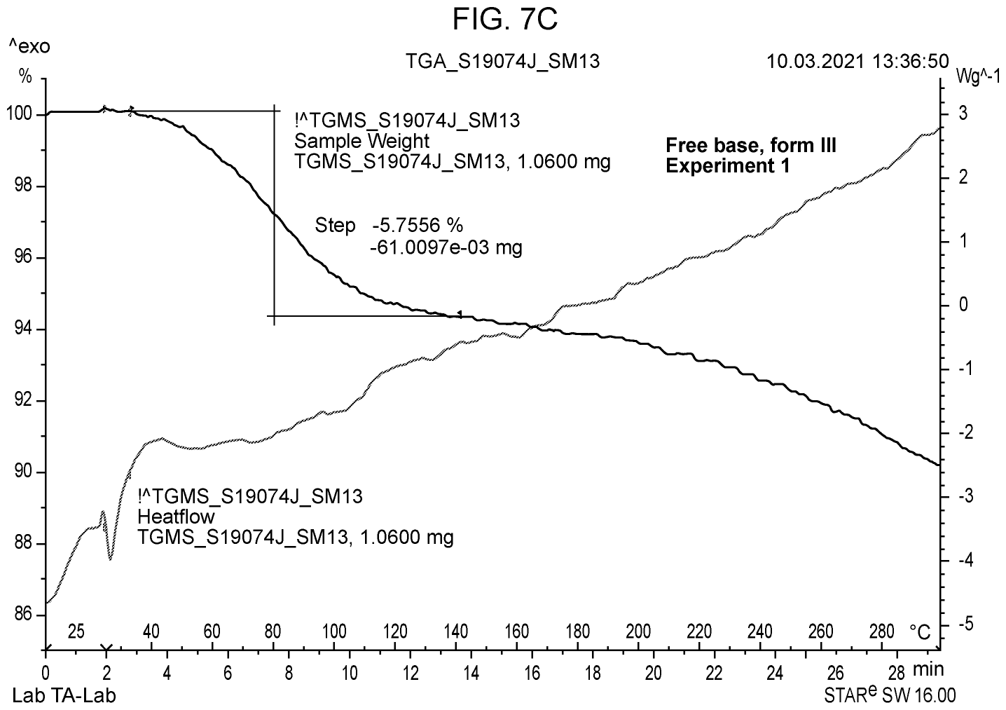

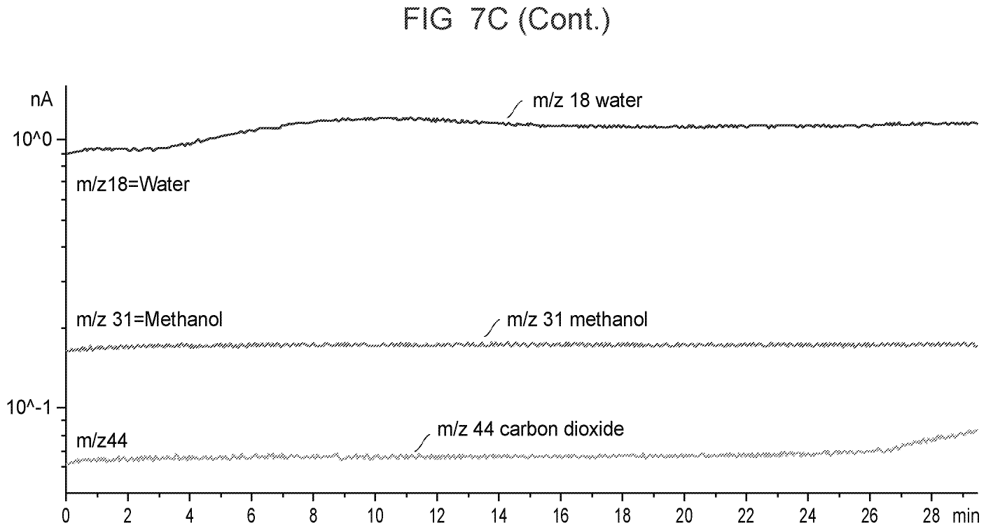

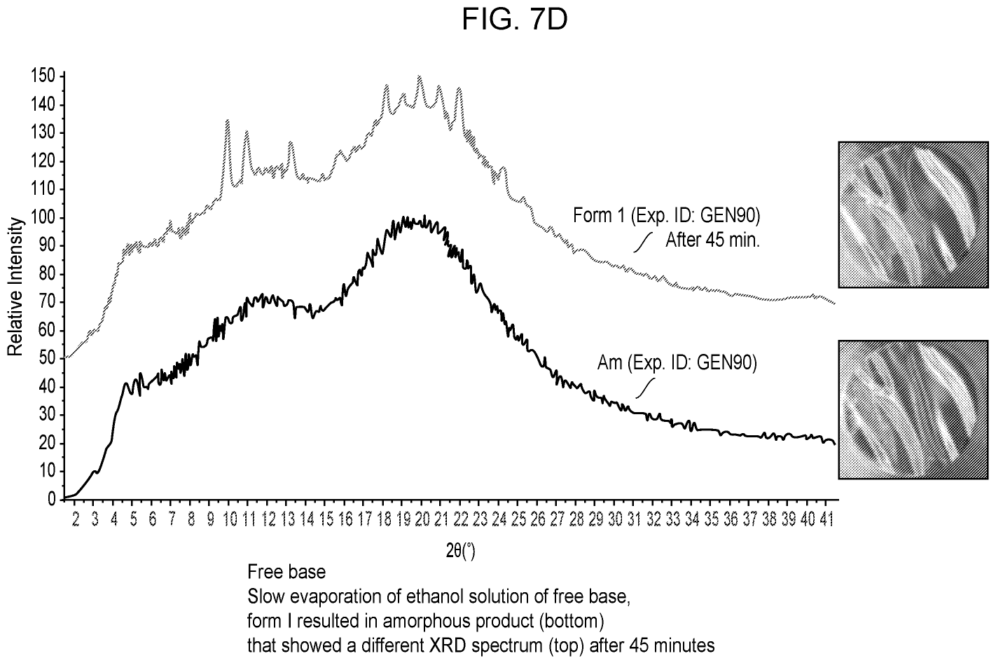

The resulting products were physically observed to be an oil, and XRD showed the product to be amorphous. However, upon conducting additional XRD characterizations after about 45 minutes, it was observed that the amorphous product began crystalizing. FIG. 7D shows the initial XRD (bottom) and the XRD after about 45 minutes (top).

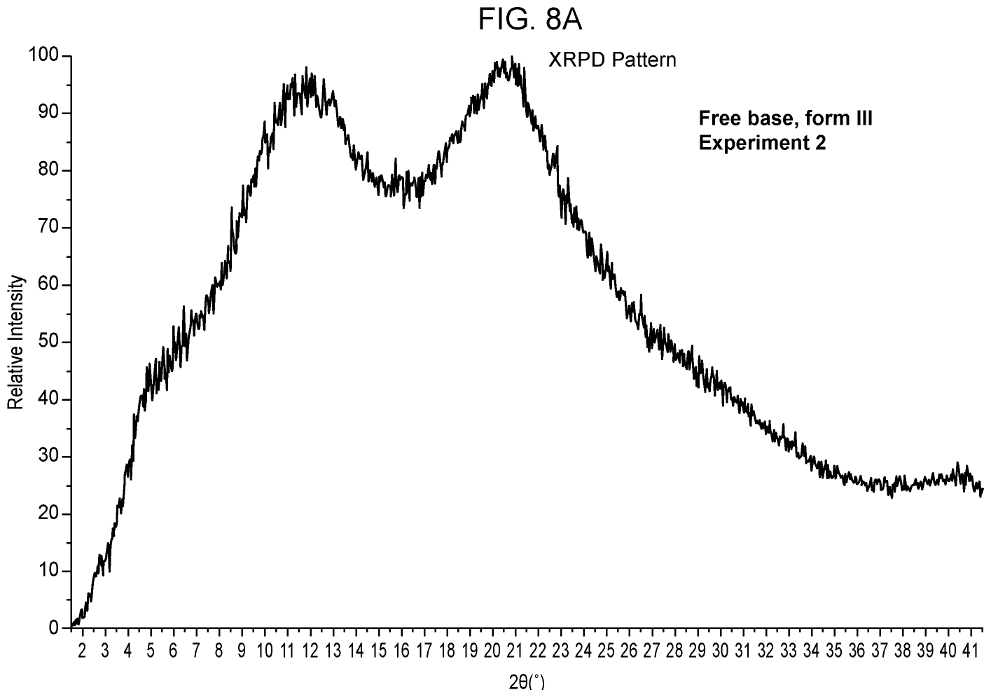

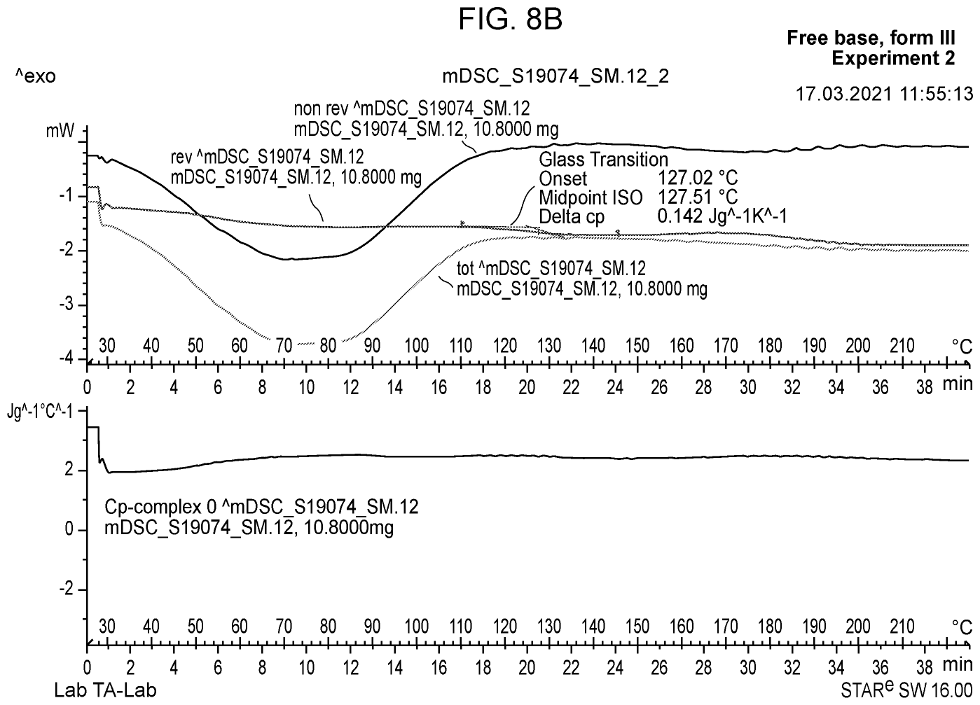

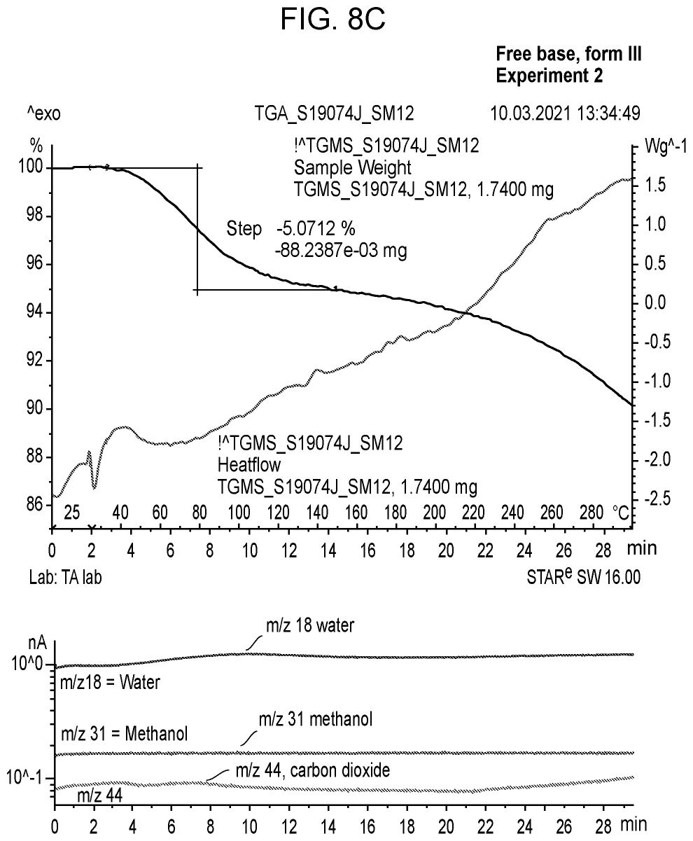

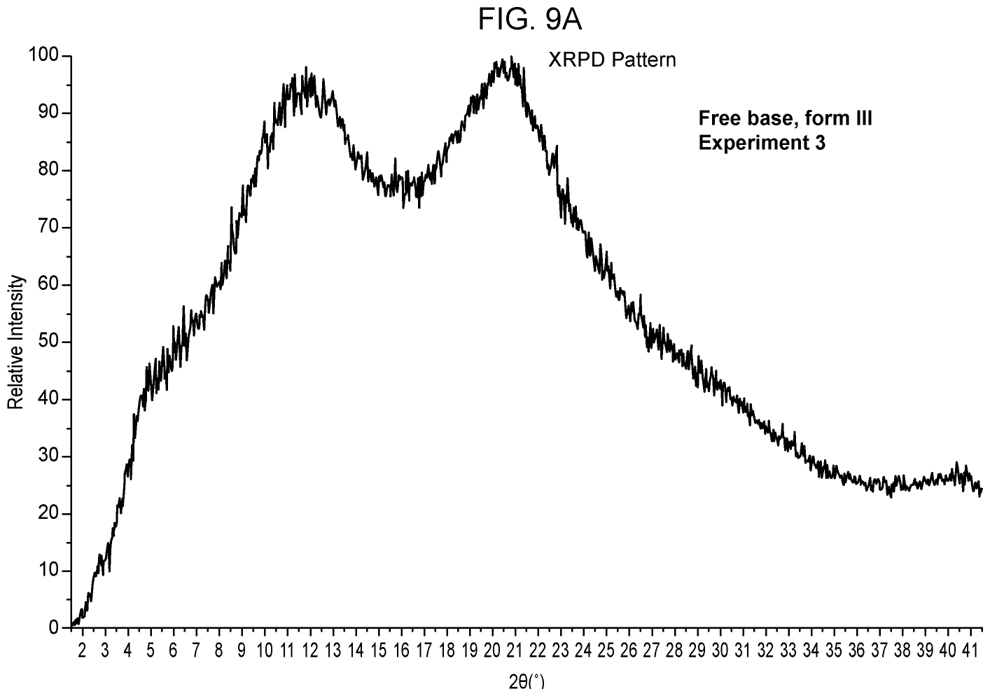

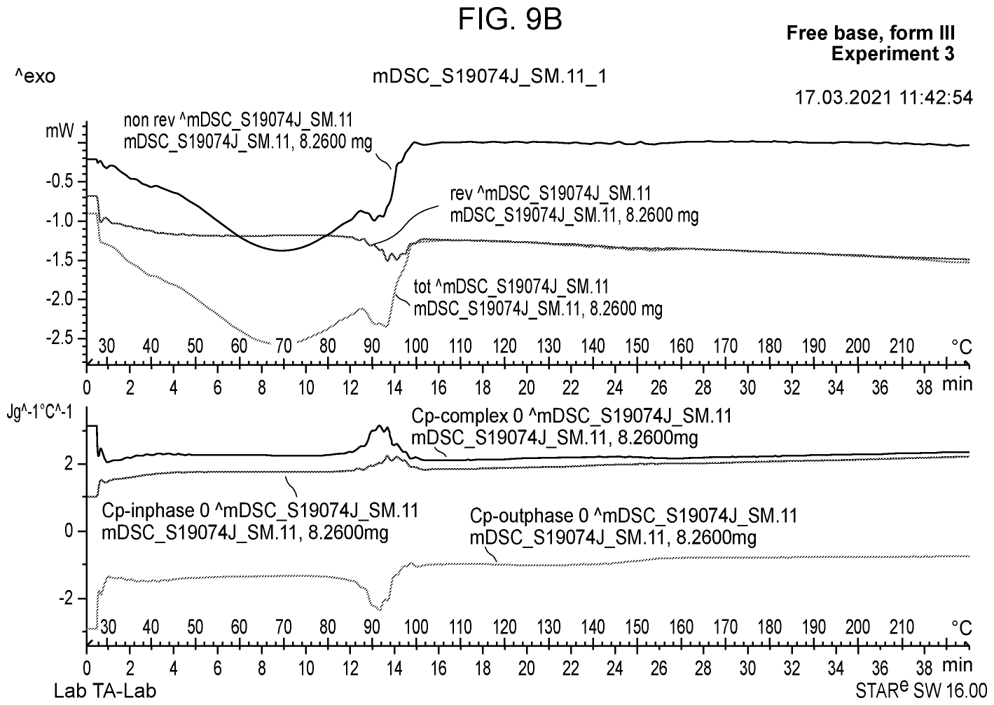

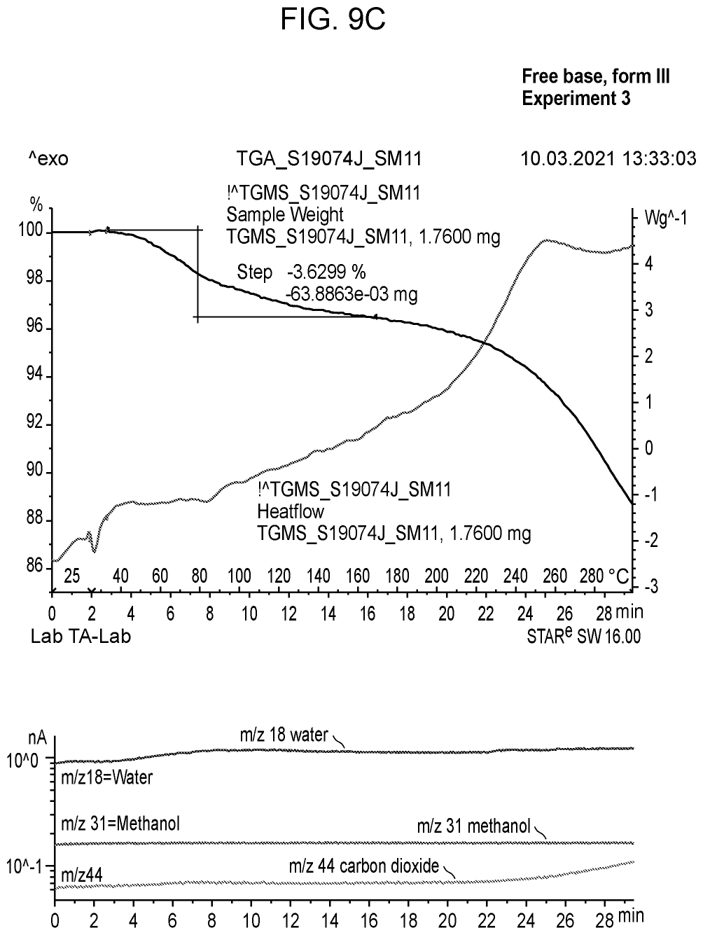

Subsequent attempts to create an amorphous form of the free base used spray drying. PVP K30 (Kollidon 30) was stirred in MeOH at room temperature until solution is obtained. The Compound A Free Base Form I was then added into the solution and stirred to dissolve. The ratio between Free Base Form I and PVP K30 in MeOH is depicted in the table below. The spray drying parameters are shown in another table below. It was found that the spray drying resulted in a crystalized form that has been assigned as Form III.

| | Experiment 1 | Experiment 2 | Experiment 3 |
|---|---|---|---|
| Free base form I | 2 | 3 | 5 |
| PVP K30 polymer | 8 | 7 | 5 |
| Methanol | 90 | 90 | 90 |
| Crystallization method | Spray drying | Spray drying | Spray drying |
| XRPD Outcome | Form III | Form III | Form III |
| Characterization | FIGS. 7A-C | FIGS. 8A-C | FIGS. 9A-C |

** values are in % w/w

| | |
|---|---|
| Air speed (m³/min) | 0.6 |
| Air temperature (C) | 70 |
| Chamber out temperature (C) | 45.2 |
| Air out temperature (C) | 39.1 |
| UP chamber (mbar) | 4.6 |
| DP cyclone (mbar) | 22.1 |
| Air nozzle (L/min) | 7.0 |
| Cyclone air (L/min) | 100 |
| Pump speed (rpm) | 12 (approximately 9 g/min) |

### Example 2

### Tosylate Salts of Compound A

Synthesis of the tosylate salts began by mixing form I of the free base and 1M aqueous solutions of the counterion (tosylate) in water/t-butanol (1:9, v/v). This mixture was then frozen in liquid nitrogen and freeze-dried for 18 hours with a freeze dryer (Alpha 2-4 LD, Martin Christ).

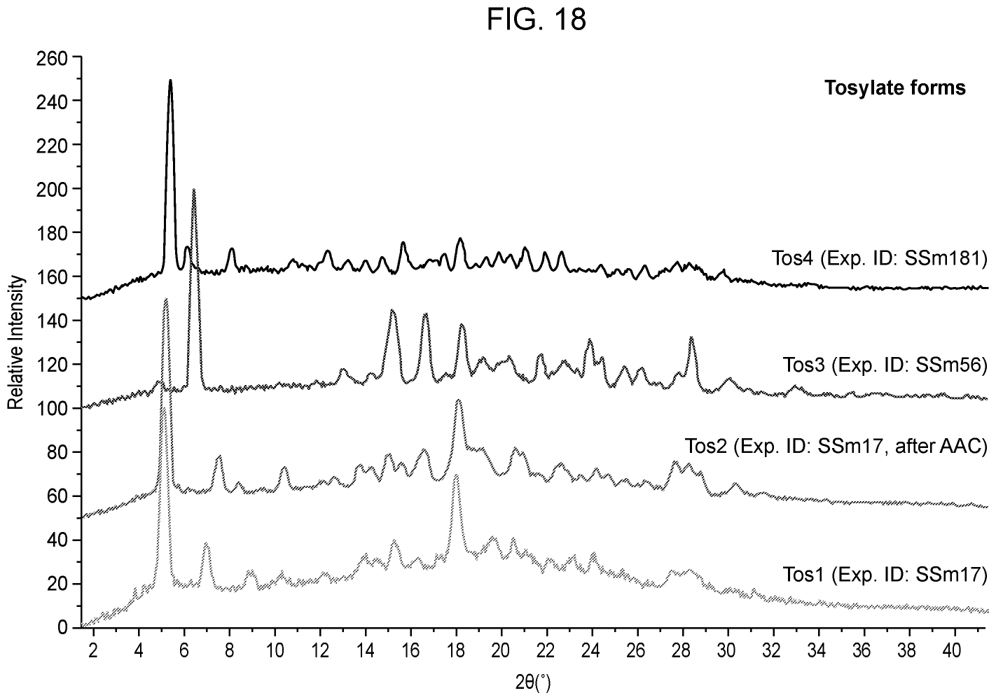

Next, the resulting solids were combined with another solvent (acetonitrile (AcN) or ethanol) and the resulting mixtures were stirred at room temperature (RT). After stirring, the solids were isolated by centrifugation and part of the solids was harvested on a 96-well plate and dried under ambient conditions. The remaining part of the solids was dried under vacuum (5 mbar [i.e. 500 Pa], RT, 18 h). All solids were analyzed by XRPD, subjected to accelerated aging conditions (AAC; 40° C./75% RH) and reanalyzed by XRPD. The procedure and resulting tosylate forms are shown in the table below, wherein forms were identified by XRD. Characterizations of the forms is shown in FIGS. 10-18.

| | Conc | Ambient | Freeze | 3 days | 5 days |
|---|---|---|---|---|---|
| Solvent | (mg/ml) | Evap | Dry | AAC | AAC |
| Acetonitrile | 200 | Form IV | Form IV | Forms I, | Form |
| | | | | II, III | III |
| Acetonitrile | 750 | Amorphous | Amorphous | Form II | Form II |
| Ethanol | 312 | Form III | Form III | — | — |
| Ethanol | 348 | Form III | Form III | — | — |

### Example 3

### Oxalate Salts of Compound A

The oxalate salts were generated in a similar manner to the tosylate salts.

Synthesis of the oxalate salts began by mixing form I of the free base and 1M aqueous solutions of the counterion (oxalate) in water/t-butanol (1:9, v/v). This mixture was then frozen in liquid nitrogen and freeze-dried for 18 hours with a freeze dryer (Alpha 2-4 LD, Martin Christ).

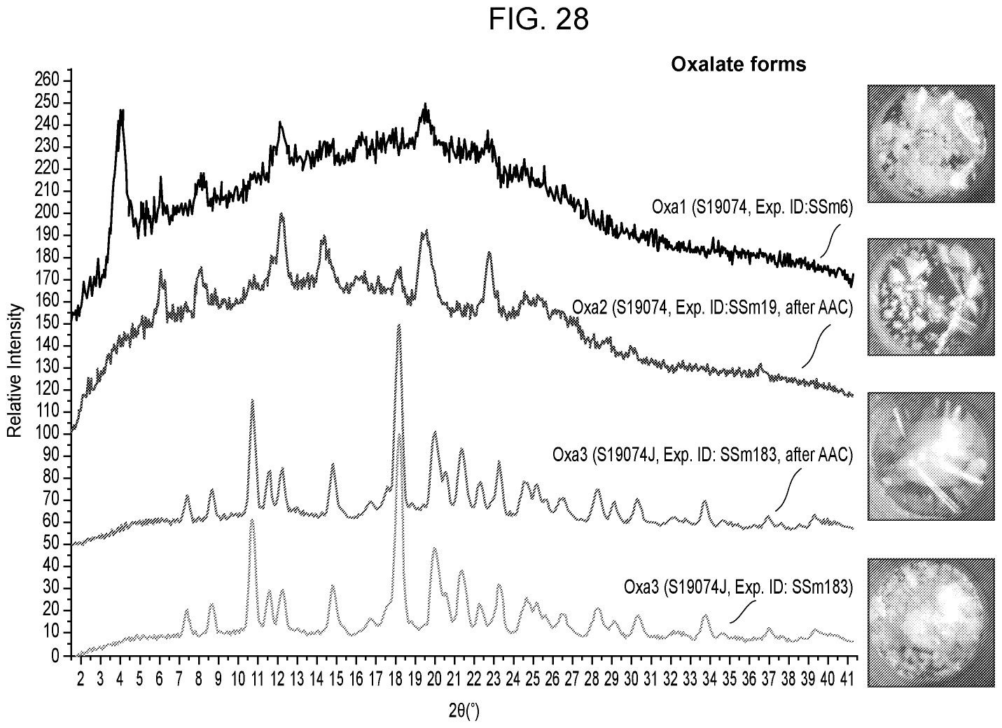

Next, the resulting solids were combined with another solvent (acetonitrile (AcN) or chloroform) and the resulting mixtures were stirred at room temperature (RT). After stirring, the solids were isolated by centrifugation and part of the solids was harvested on a 96-well plate and dried under ambient conditions. The remaining part of the solids was dried under vacuum (5 mbar, RT, 18 h). All solids were analyzed by XRPD, subjected to AAC (accelerated aging conditions; 40° C./75% RH) and reanalyzed by XRPD. The methods and results are summarized in the table below, and the characterizations are shown in FIGS. 19-28.

| | Counterion | Conc | Ambient | Freeze | 3 days | 5 days |
|---|---|---|---|---|---|---|
| Solvent | Ratio | (mg/ml) | Evaporation | Dry | AAC | AAC |
| Acetonitrile | 1.1 | 200 | Form I | — | — | — |
| Chloroform | 2 | 200 | Form II | Form II | Form II | Form II |
| Acetonitrile | 2 | 100 | Form III | Form III | — | — |
| Acetonitrile | 2 | 103 | Form II, III | Form III | — | — |

### Example 4

### Fumarate Salts of Compound A

The fumarate salts were generated in a similar manner to the tosylate salts.

Synthesis of the fumarate salts began by mixing form I of the free base and 1M aqueous solutions of the counterion (fumarate) in water/t-butanol (1:9, v/v). This mixture was then frozen in liquid nitrogen and freeze-dried for 18 hours with a freeze dryer (Alpha 2-4 LD, Martin Christ).

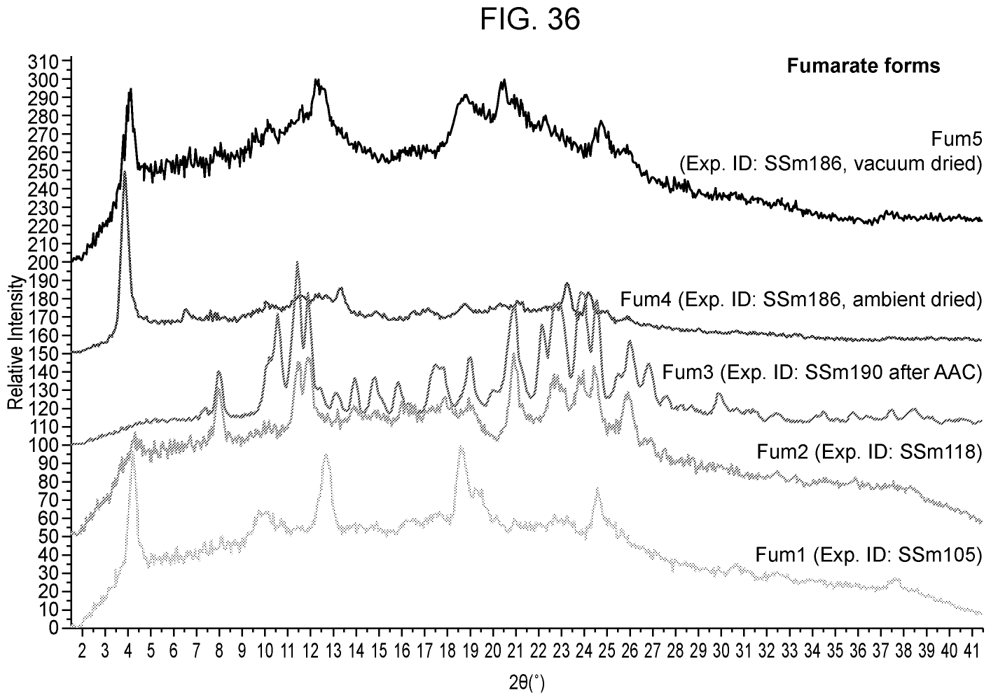

Next, the resulting solids were combined with another solvent (AcN, diethyl ether, or toluene), and the resulting mixtures were stirred at room temperature (RT). After stirring, the solids were isolated by centrifugation and part of the solids was harvested on a 96-well plate and dried under ambient conditions. The remaining part of the solids was dried under vacuum (5 mbar, RT, 18 h). All solids were analyzed by XRPD, subjected to AAC (40° C./75% RH) and reanalyzed by XRPD. The methods and results are summarized in the table below, and the characterizations are shown in FIGS. 29-36.

| | Conc | Ambient | Freeze | 3 days | 5 days |
|---|---|---|---|---|---|
| Solvent | (mg/ml) | Evaporation | Dry | AAC | AAC |
| Acetonitrile | 200 | Form I | — | — | — |
| Diethyl ether, | 200 | Form II | — | Form III | — |
| chloroform, | | | | | |
| or toluene | | | | | |
| Acetonitrile | 97 | Form IV | Form V | Form III | — |
| Acetonitrile | 103 | — | — | Form III | — |

### Example 5

### Camsylate Salts of Compound A

The camsylate salts were generated in a similar manner to the tosylate salts.

Synthesis of the camsylate salts began by mixing form I of the free base and 1M aqueous solutions of the counterion (camsylate) in water/t-butanol (1:9, v/v). This mixture was then frozen in liquid nitrogen and freeze-dried for 18 hours with a freeze dryer (Alpha 2-4 LD, Christ).

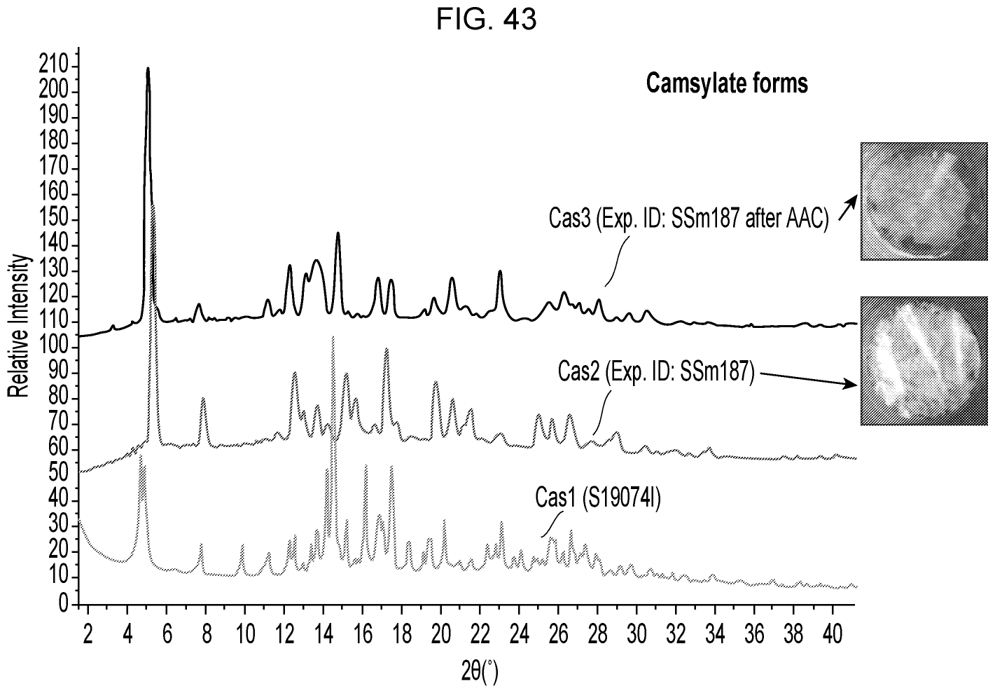

Next, the resulting solids were combined with another solvent (1,4-dioxane, acetonitrile, ethyl acetate), and the resulting mixtures were stirred at room temperature (RT). After stirring, the solids were isolated by centrifugation and part of the solids was harvested on a 96-well plate and dried under ambient conditions. The remaining part of the solids was dried under vacuum (5 mbar, RT, 18 h). All solids were analyzed by XRPD, subjected to AAC (40° C./75% RH) and reanalyzed by XRPD. The methods and results are summarized in the table below, and the characterizations are shown in FIGS. 37-43.

| | Conc | Ambient | Freeze | 3 days | 5 days |
|---|---|---|---|---|---|
| Solvent | (mg/ml) | Evaporation | Dry | AAC | AAC |
| 1,4-dioxane | 151 | Form II | Form II | Form II | — |
| Acetonitrile | 438 | Form II | Form II | Form II | Form III |
| Ethyl acetate | 71 | Form II | Form II | Form II | Form III |
| Acetonitrile | 438 | Form II | Form II | Form II | — |

Form I (single crystal) was prepared by dissolving 20-30 mg Compound A into 100 μl dioxane followed by addition of camsylate counterion in 300 μl dioxane. After solution is obtained, the mixture was left open for slow evaporation.

### Example 6

### Citrate Salts of Compound A

The citrate salts were generated in a similar manner to the tosylate salts.

Synthesis of the citrate salts began by mixing form I of the free base and 1M aqueous solutions of the counterion (citrate) in water/t-butanol (1:9, v/v). This mixture was then frozen in liquid nitrogen and freeze-dried for 18 hours with a freeze dryer (Alpha 2-4 LD, Christ).

Next, the resulting solids were combined with another solvent (acetonitrile or ethyl acetate), and the resulting mixtures were stirred at room temperature (RT). After stirring, the solids were isolated by centrifugation and part of the solids was harvested on a 96-well plate and dried under ambient conditions. The remaining part of the solids was dried under vacuum (5 mbar, RT, 18 h). All solids were analyzed by XRPD, subjected to AAC (40° C./75% RH) and reanalyzed by XRPD. The methods and results are summarized in the table below, and the characterizations are shown in FIGS. 44-46.

| | Conc | Ambient | Freeze | 3 days | 5 days |
|---|---|---|---|---|---|
| Solvent | (mg/ml) | Evaporation | Dry | AAC | AAC |
| Acetonitrile | 53 | Form I | Form I | — | — |
| Ethyl acetate | 52 | Form I | Form I | — | — |
| Acetonitrile | 103 | Form I | Form I | — | — |

### Example 7

### Hydrochloride Salts of Compound A

The hydrochloride salts were generated in a similar manner to the tosylate salts.

Synthesis of the hydrochloride salts began by mixing form I of the free base and 1M aqueous solutions of the counterion (chloride) in water/t-butanol (1:9, v/v). This mixture was then frozen in liquid nitrogen and freeze-dried for 18 hours with a freeze dryer (Alpha 2-4 LD, Christ).

Next, the resulting solids were combined with another solvent (acetonitrile or diethyl ether), and the resulting mixtures were stirred at room temperature (RT). After stirring, the solids were isolated by centrifugation and part of the solids was harvested on a 96-well plate and dried under ambient conditions. The remaining part of the solids was dried under vacuum (5 mbar, RT, 18 h). All solids were analyzed by XRPD, subjected to AAC (40° C./75% RH) and reanalyzed by XRPD. The methods and results are summarized in the table below, and the characterizations are shown in FIGS. 47-53.

| | Conc | Counterion | Ambient | Freeze | 3 days | 5 days |
|---|---|---|---|---|---|---|
| Solvent | (mg/ml) | Ratio | Evaporation | Dry | AAC | AAC |
| Acetonitrile | 200 | 2.2 | Form II | — | Changed | — |
| | | | | | to oil-like | |
| Diethyl ether | 200 | 2.2 | Form III | — | Changed | — |
| | | | | | to poorly | |
| | | | | | crystalline | |

### Example 8

Napthalene-1,5-disulfonic acid Salts of Compound A

The napthalene-1,5-disulfonic acid salts were prepared by dissolving 20-30 mg compound I in 100 μL water in 8 ml vial. One equivalent of counterion (Nds) was added in 300 μL of water solution at room temperature. The vial was sealed and shaken for 30 seconds before it was left open for several days to allow for evaporation. The solid was characterized by XRD, as shown in FIG. 54.

### Example 9

### Solubility of Free Base and Salt Forms of Compound A

As shown in the table below, the salts of Compound A exhibit varying degree of improved solubility in physiological conditions, making them suitable for formulation development of different immediate release and modified release oral dosage formulations. FaSSGF is Fasted State Simulated Gastric Fluid. Solubility is reported in mg/mL.

| | | pH | Solubility | Solubility | pH | pH | pH |
|---|---|---|---|---|---|---|---|
| Form | Medium | Buffer | (2 h) | (24 h) | (30 min) | (2 h) | (24 h) |
| Free base I | Water | | | 5.1 | | | 10.1 |
| Free base I | FaSSGF | 7.4 | | 5.9 | | | 10.2 |
| Free base I | 50 mM | 1.6 | | 9.8 | | | 9.4 |
| | phosphate | | | | | | |
| Tosylate III | FaSSGF | 1.6 | >129 | >129 | 1.0 | 1.9 | 2.1 |
| Oxalate III | FaSSGF | 1.6 | >392 | >392 | 1.6 | 1.6 | 1.5 |
| Fumarate III | FaSSGF | 1.6 | >48 | >48 | 4.1 | 1.6 | 1.7 |
| Camsylate II | FaSSGF | 1.6 | >380 | >380 | 1.6 | 1.7 | 1.7 |
| Citrate I | FaSSGF | 1.6 | >127 | >127 | 4.4 | 1.6 | 1.6 |
| Tosylate III | 100 mM | 6.8 | 8.7 | 2.92 | 2.7 | 6.8 | 7.0 |
| | phosphate | | | | | | |
| Oxalate III | 100 mM | 6.8 | 55.5 | 38.1 | 2.1 | 6.7 | 6.8 |
| | phosphate | | | | | | |
| Fumarate III | 100 mM | 6.8 | >107 | >107 | 4.5 | 7.2 | 7.3 |
| | phosphate | | | | | | |
| Camsylate II | 100 mM | 6.8 | 28.9 | 42.2 | 4.1 | 7.4 | 7.5 |
| | phosphate | | | | | | |
| Citrate I | 100 mM | 6.8 | >137 | >137 | 4.5 | 6.7 | 6.7 |
| | phosphate | | | | | | |

As shown above, it was observed that solubility of the free base in water was 5.9 mg/ml, whereas the solubility was increased for the tosylate, oxalate, fumarate, camsylate, and citrate forms studied.

### Clauses

Notwithstanding the appended claims, the disclosure is also defined by the following clauses:
1. A polymorph of a free base of formula (I):

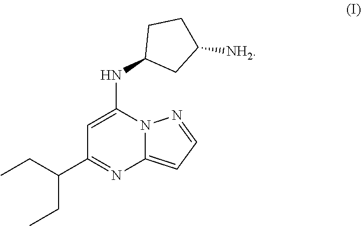 [SMILES: CCC(CC)c1cc(N[C@H]2CC[C@H](N)C2)n2nccc2n1]

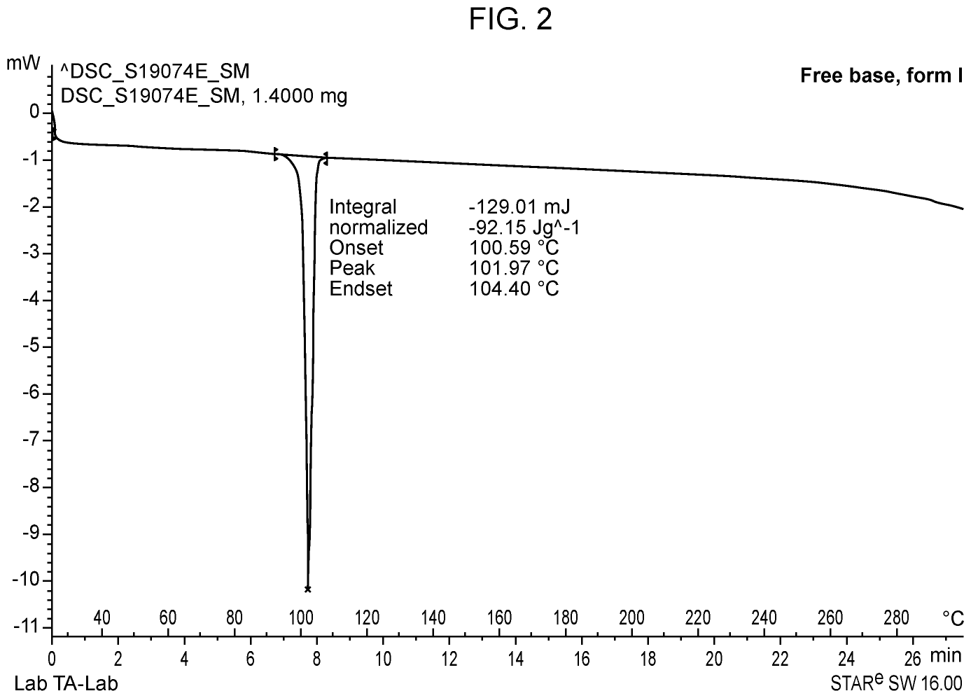

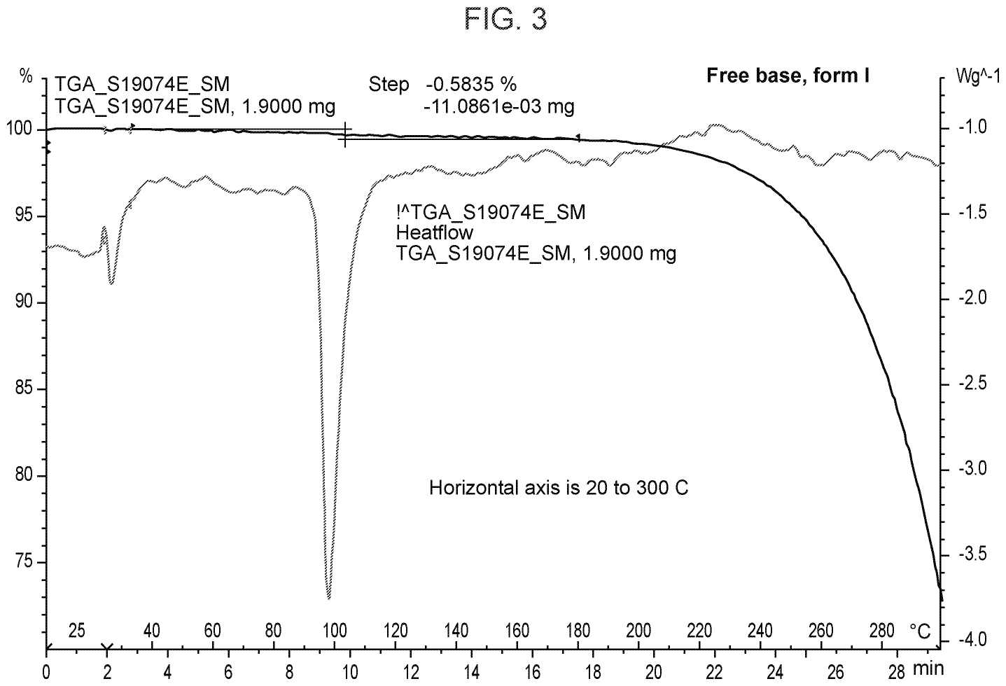

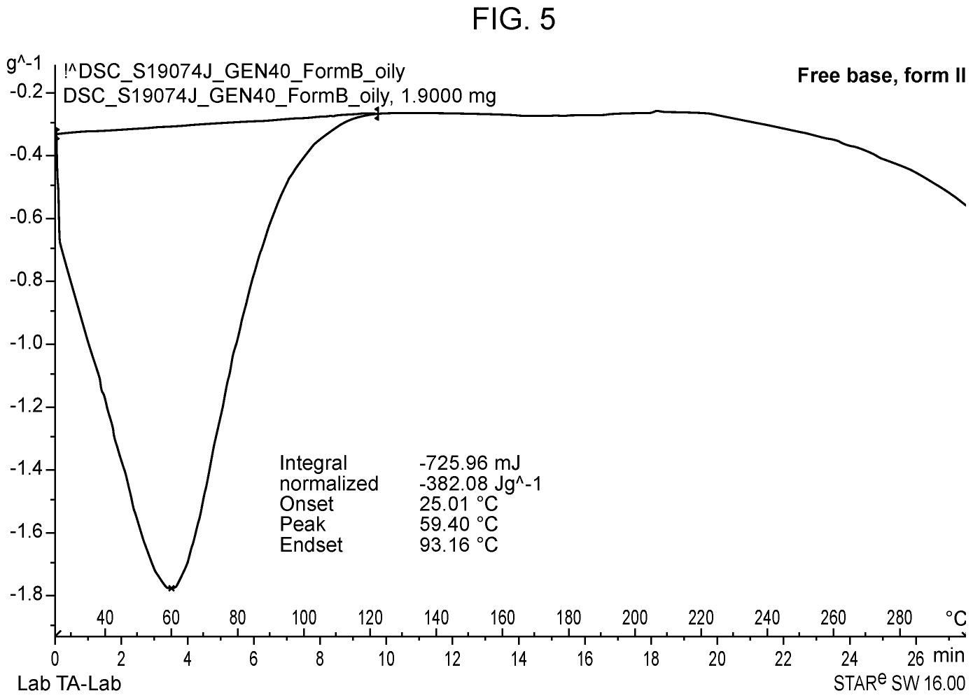

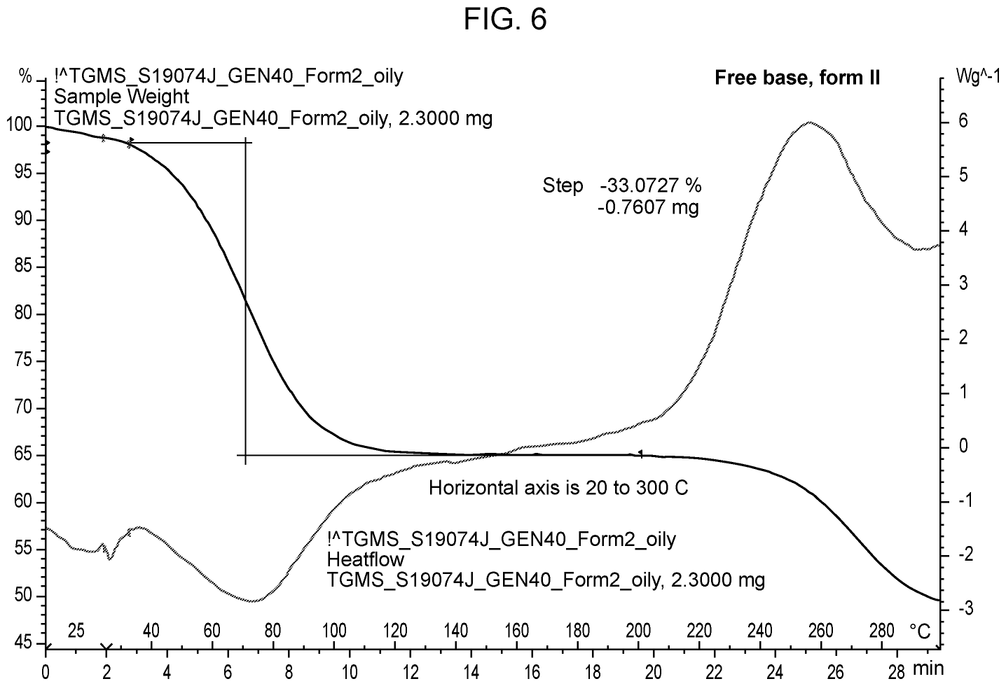

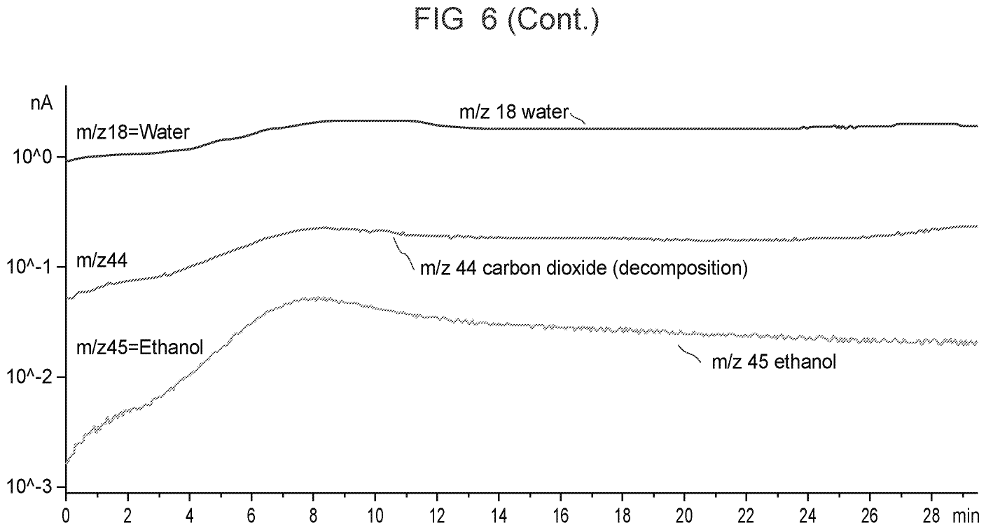

2. The polymorph of clause 1, wherein the polymorph has form I.
3. The polymorph of clause 2, characterized by an X-ray diffraction (XRD) pattern comprising 2θ peaks, ±0.2 degrees 2θ, at 10.0, 11.0, 13.3, 18.2, and 22.0.
4. The polymorph of any one of clauses 2-3, characterized by an XRD pattern substantially as shown in FIG. 1.
5. The polymorph of any one of clauses 2-4, characterized by a differential scanning calorimetry (DSC) thermogram substantially as shown in FIG. 2.
6. The polymorph of any one of clauses 2-5, characterized by a thermogravimetric analysis coupled to mass spectrometry (TGA-MS) spectrum substantially as shown in FIG. 3.
7. The polymorph of clause 1, wherein the polymorph has form II.
8. The polymorph of clause 7, characterized by an XRD pattern comprising 2θ peaks, ±0.2 degrees 2θ, at 7.2, 14.4, 21.7, and 22.5.
9. The polymorph of any one of clauses 7-8, characterized by an XRD pattern substantially as shown in FIG. 4.
10. The polymorph of any one of clauses 7-9, characterized by a DSC thermogram substantially as shown in FIG. 5.
11. The polymorph of any one of clauses 7-10, characterized by a TGA-MS spectrum substantially as shown in FIG. 6.
12. The polymorph of clause 1, wherein the polymorph has form III.
13. The polymorph of clause 12, characterized by an amorphous XRD pattern.
14. The polymorph of any one of clauses 12-13, characterized by an XRD pattern substantially as shown in FIGS. 7A, 8A, or 9A.
15. The polymorph of any one of clauses 12-14, characterized by a DSC thermogram substantially as shown in FIGS. 7B, 8B, or 9B.
16. The polymorph of any one of clauses 7-10, characterized by a TGA-MS spectrum substantially as shown in FIGS. 7C, 8C, or 9C.
17. A polymorph of a tosylate salt of formula (I):

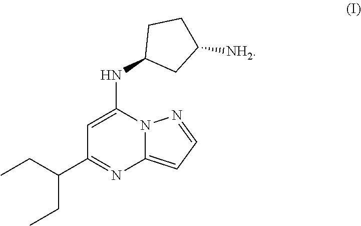 [SMILES: CCC(CC)c1cc(N[C@H]2CC[C@H](N)C2)n2nccc2n1]

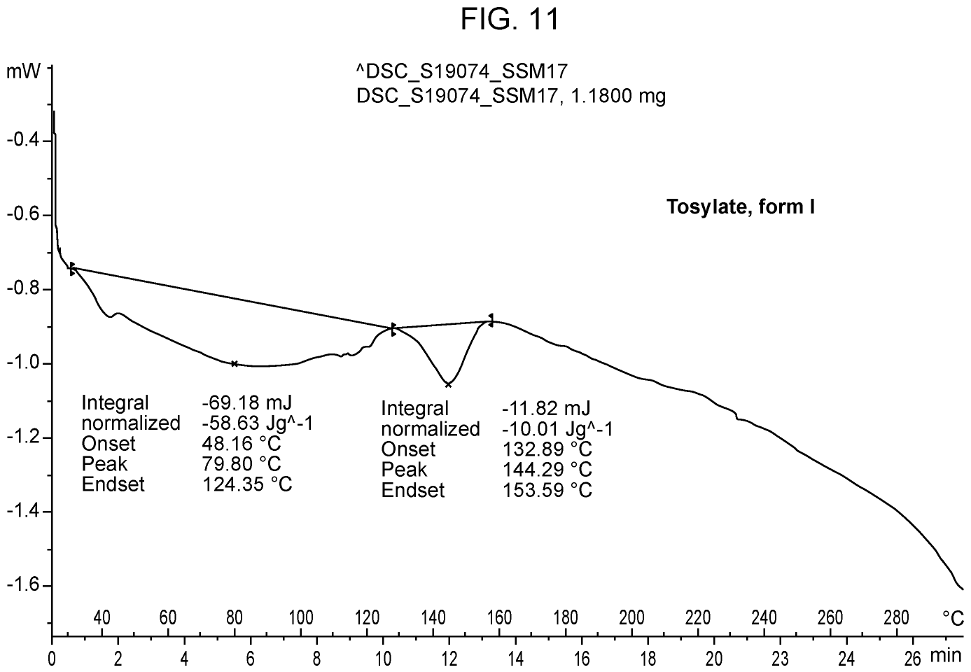

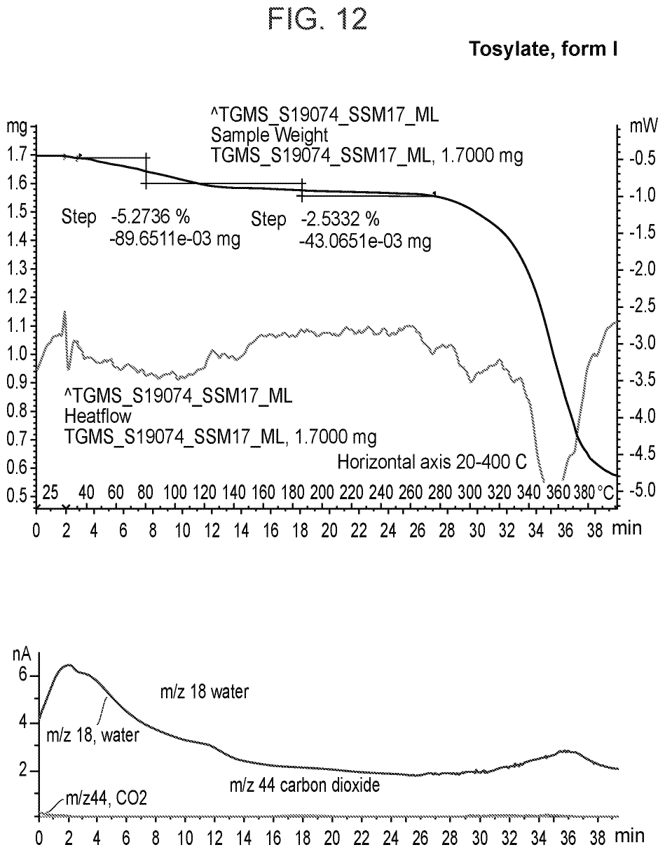

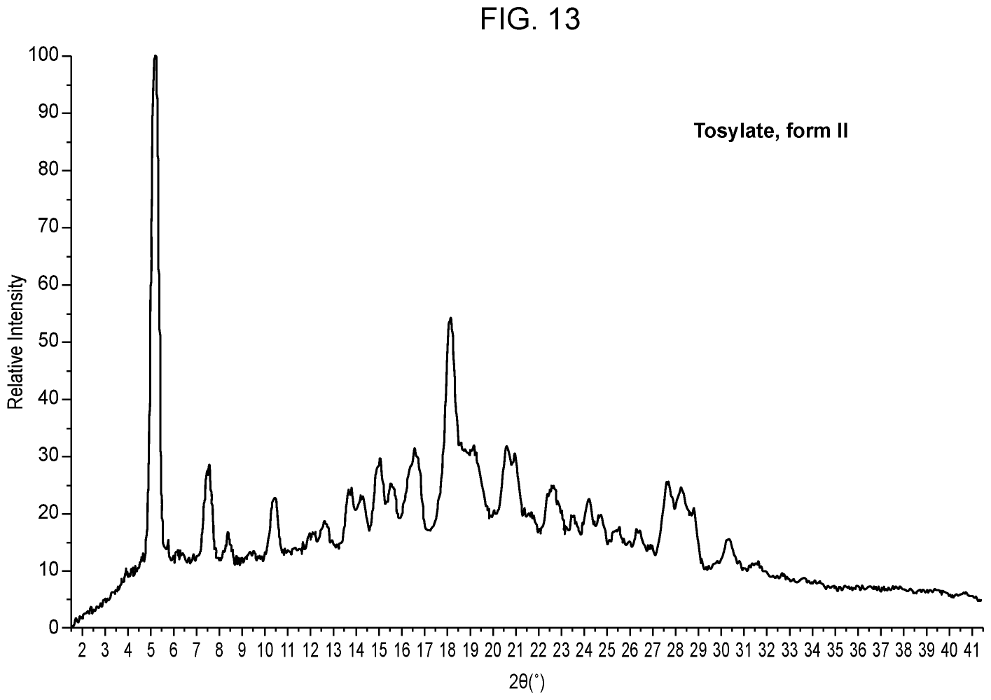

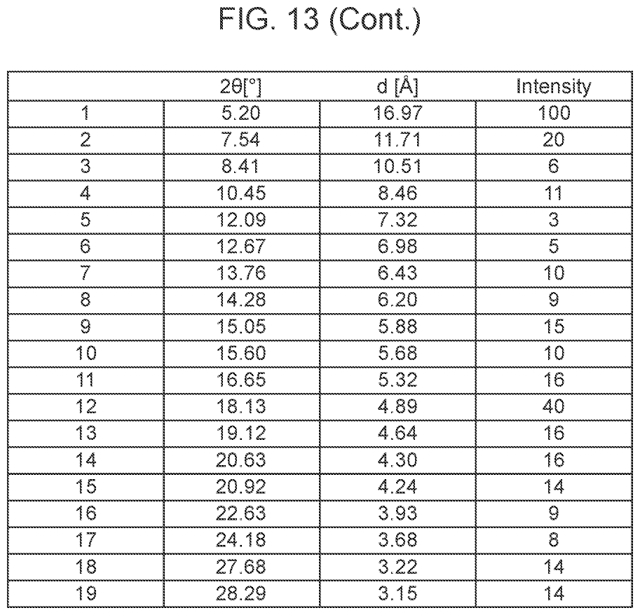

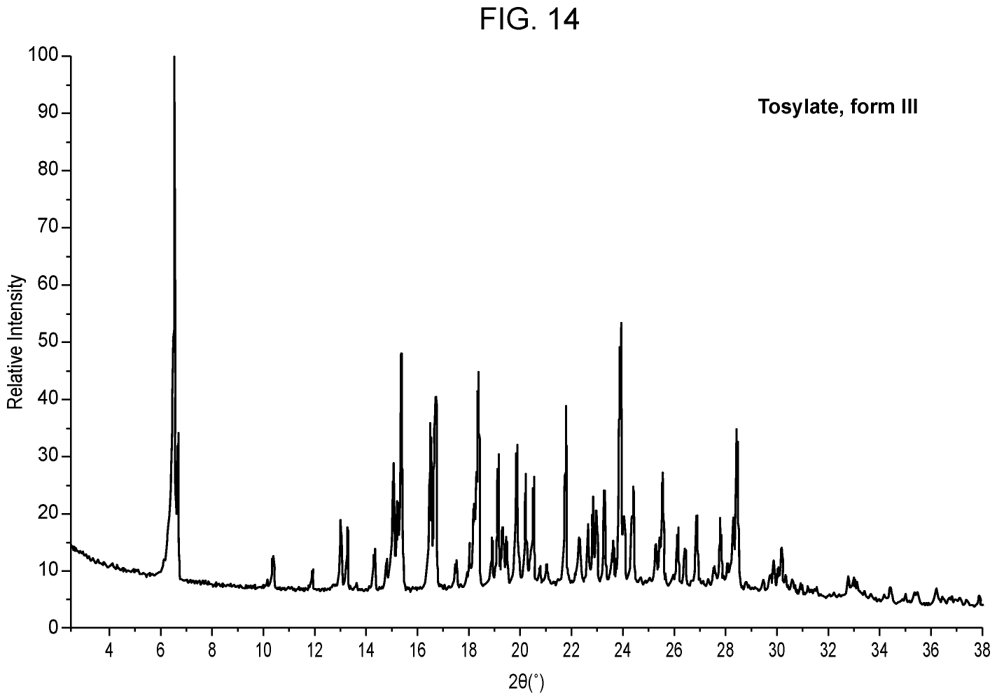

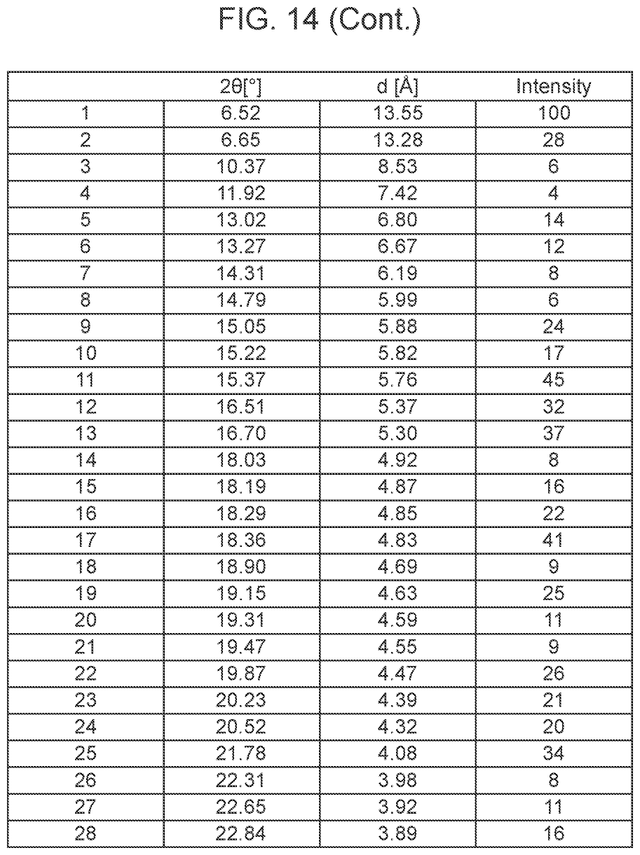

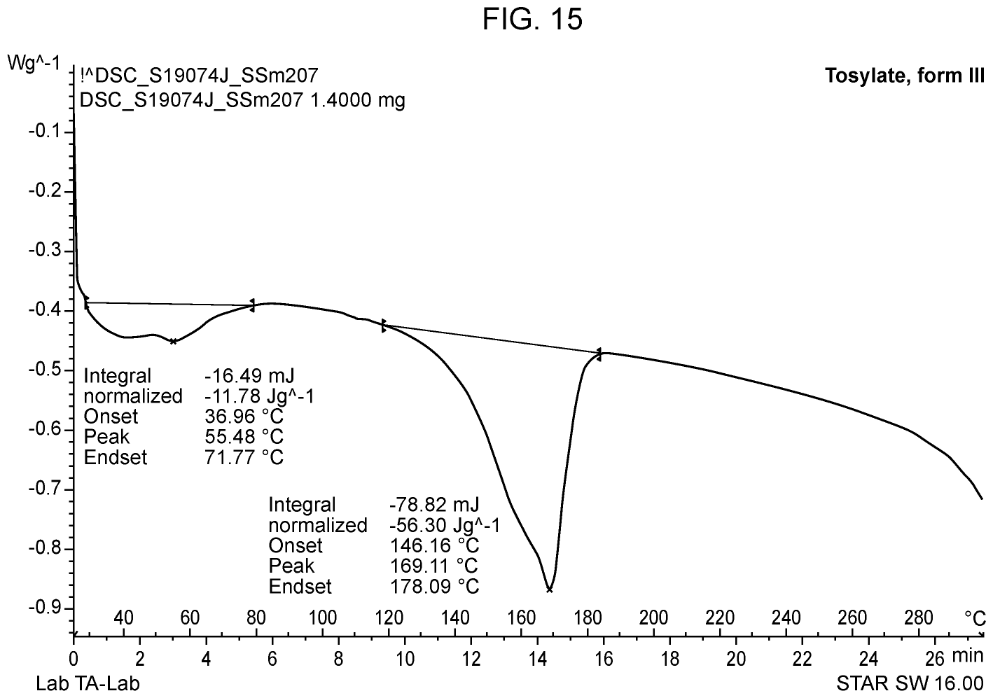

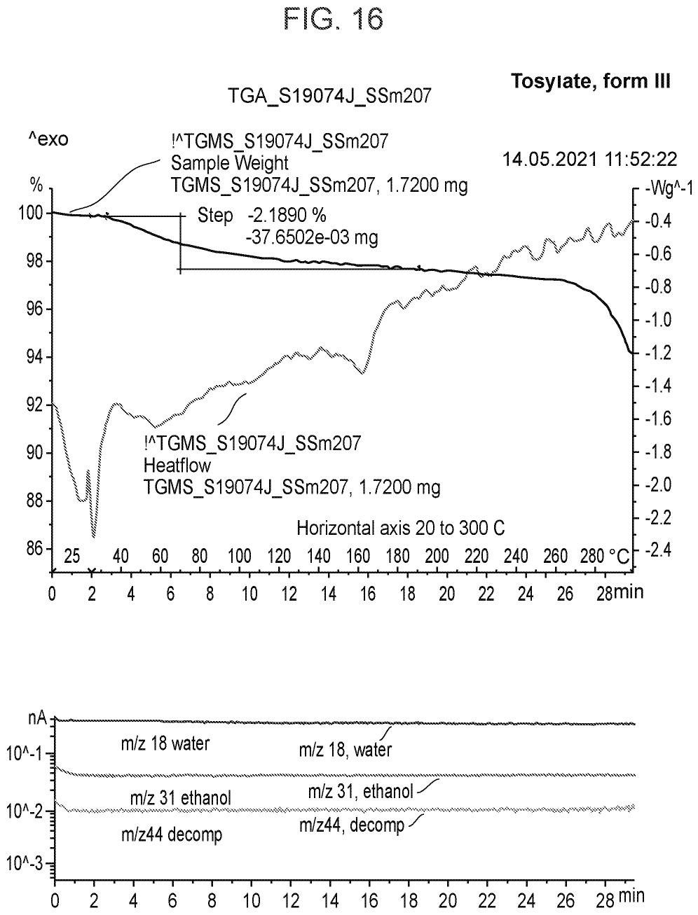

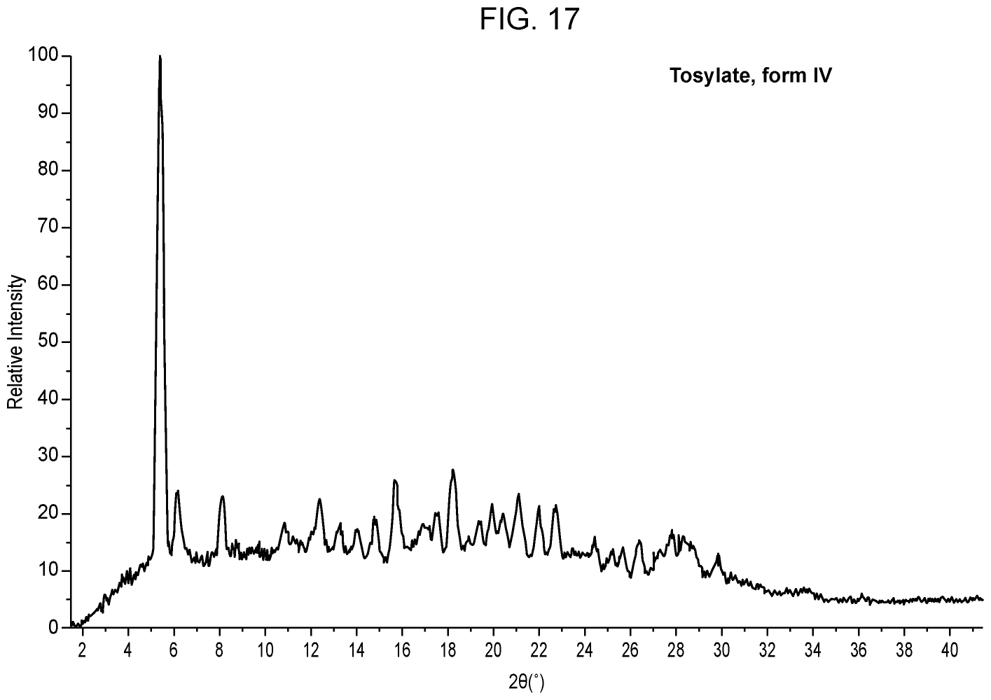

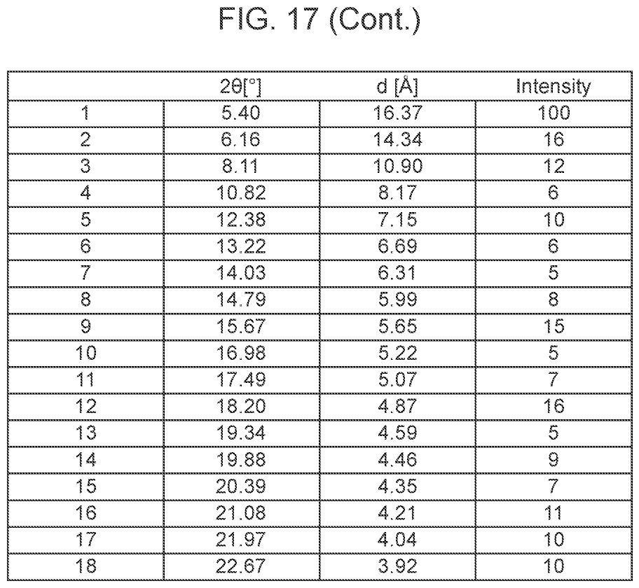

18. The polymorph of clause 17, wherein the polymorph has form I.
19. The polymorph of clause 18, characterized by an XRD pattern comprising 2θ peaks, ±0.2 degrees 2θ, at 5.1, 7.0, and 18.1.
20. The polymorph of any one of clauses 18-19, characterized by an XRD pattern substantially as shown in FIG. 10.
21. The polymorph of any one of clauses 18-20, characterized by a DSC thermogram substantially as shown in FIG. 11.
22. The polymorph of any one of clauses 18-21, characterized by a TGA-MS spectrum substantially as shown in FIG. 12.
23. The polymorph of clause 17, wherein the polymorph has form II.
24. The polymorph of clause 23, characterized by an XRD pattern comprising 2θ peaks, ±0.2 degrees 2θ, at 5.2, 7.5, 18.1, 19.1, and 20.6.
25. The polymorph of any one of clauses 23-24, characterized by an XRD pattern substantially as shown in FIG. 13.
26. The polymorph of clause 17, wherein the polymorph has form III.
27. The polymorph of clause 26, characterized by an XRD pattern comprising 2θ peaks, ±0.2 degrees 2θ, at 6.5, 15.4, 18.4, and 21.8.
28. The polymorph of any one of clauses 26-27, characterized by an XRD pattern substantially as shown in FIG. 14.
29. The polymorph of any one of clauses 26-28, characterized by a DSC thermogram substantially as shown in FIG. 15.
30. The polymorph of any one of clauses 26-29, characterized by a TGA-MS spectrum substantially as shown in FIG. 16.
31. The polymorph of clause 17, wherein the polymorph has form IV.
32. The polymorph of clause 31, characterized by an XRD pattern comprising 2θ peaks, ±0.2 degrees 2θ, at 5.4, 6.2, 15.7, and 18.2.
33. The polymorph of any one of clauses 31-32, characterized by an XRD pattern substantially as shown in FIG. 17.
34. A polymorph of an oxalate salt of formula (I):

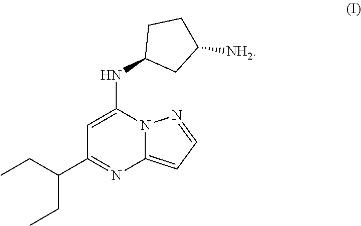 [SMILES: CCC(CC)c1cc(N[C@H]2CC[C@H](N)C2)n2nccc2n1]

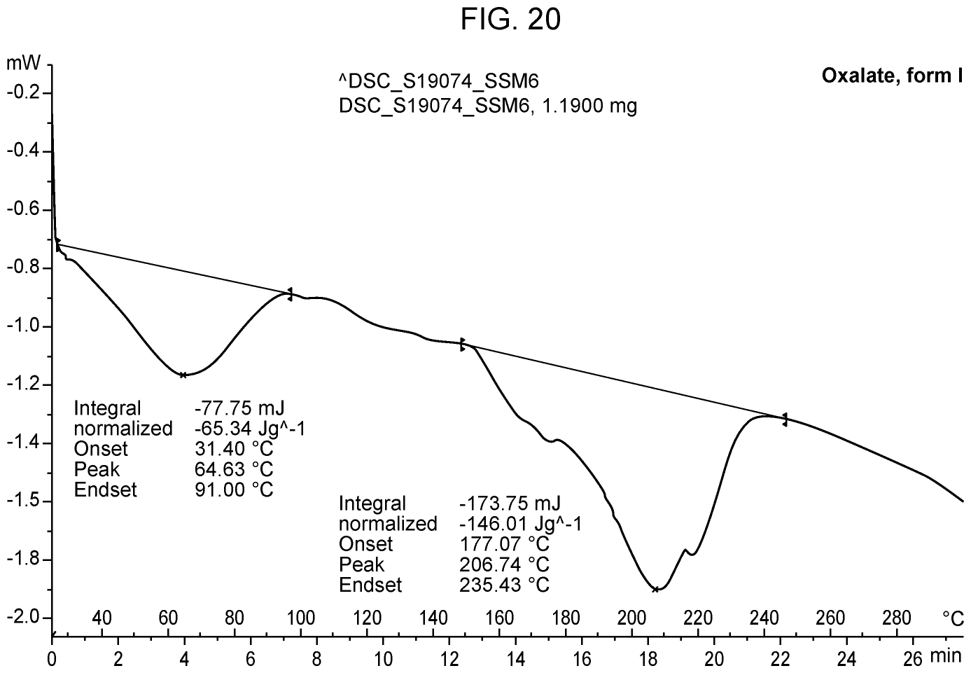

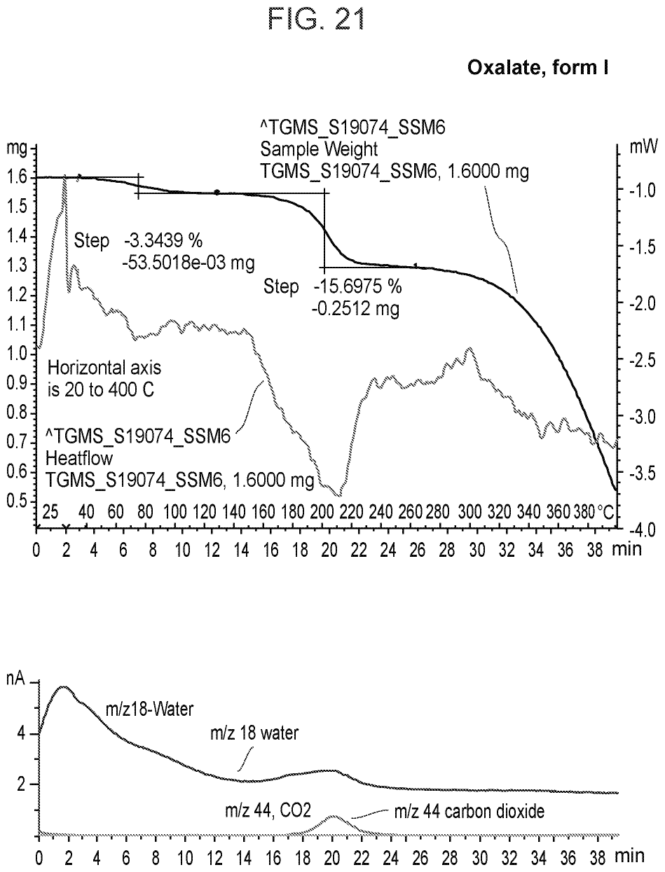

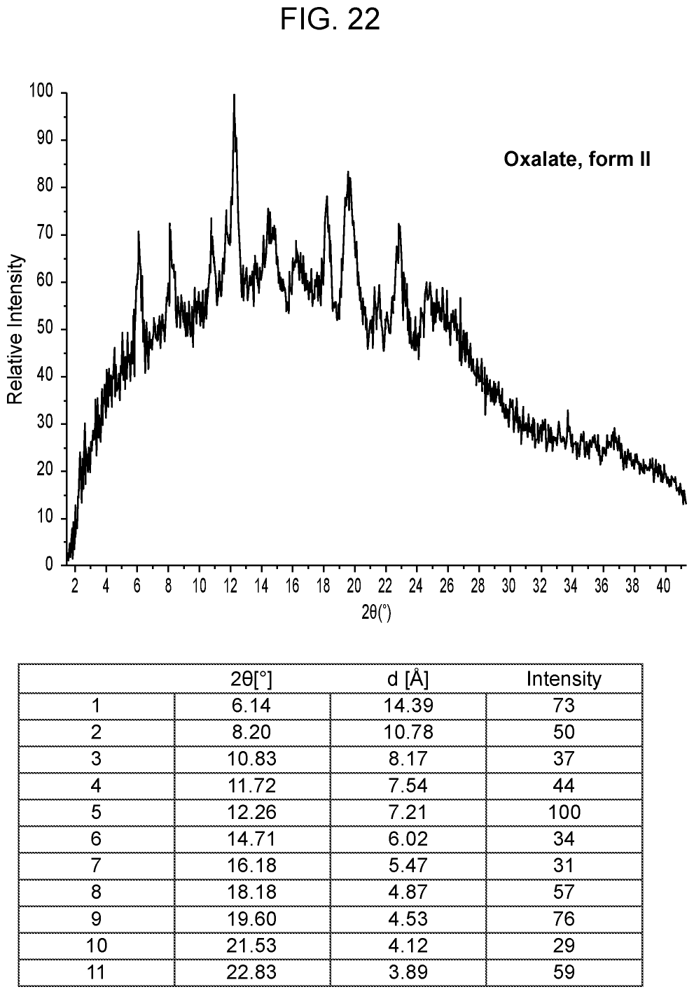

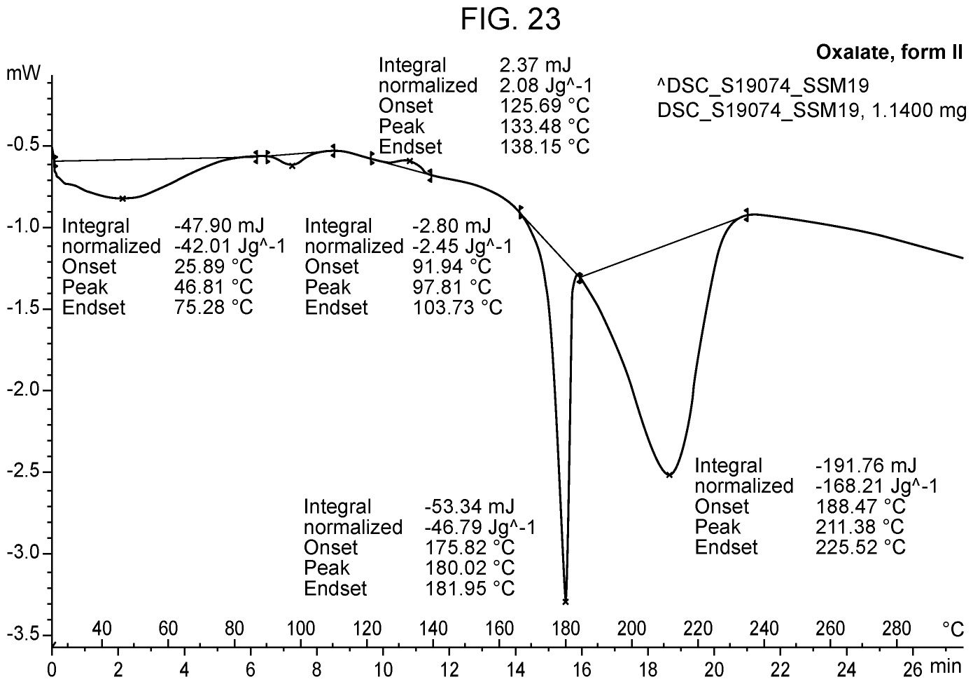

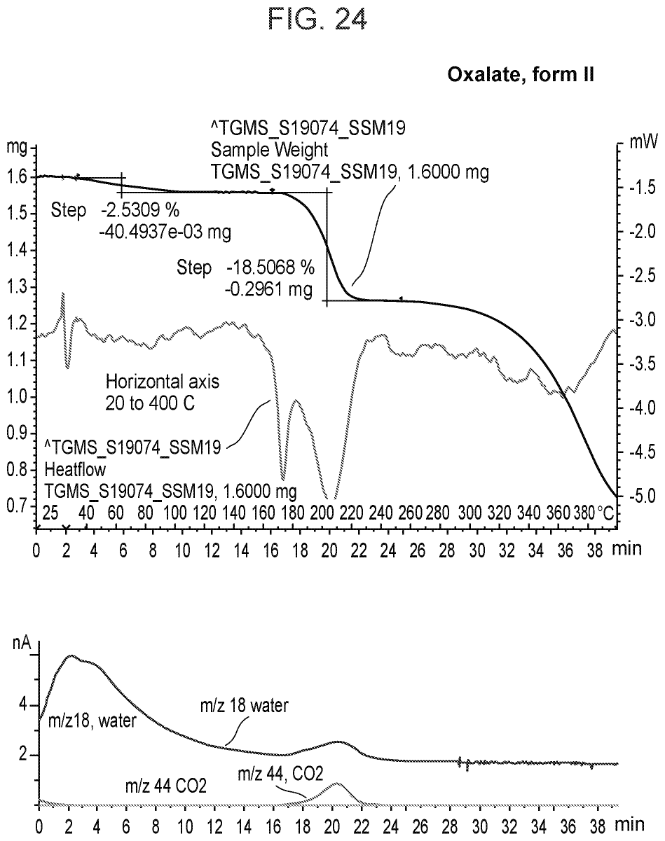

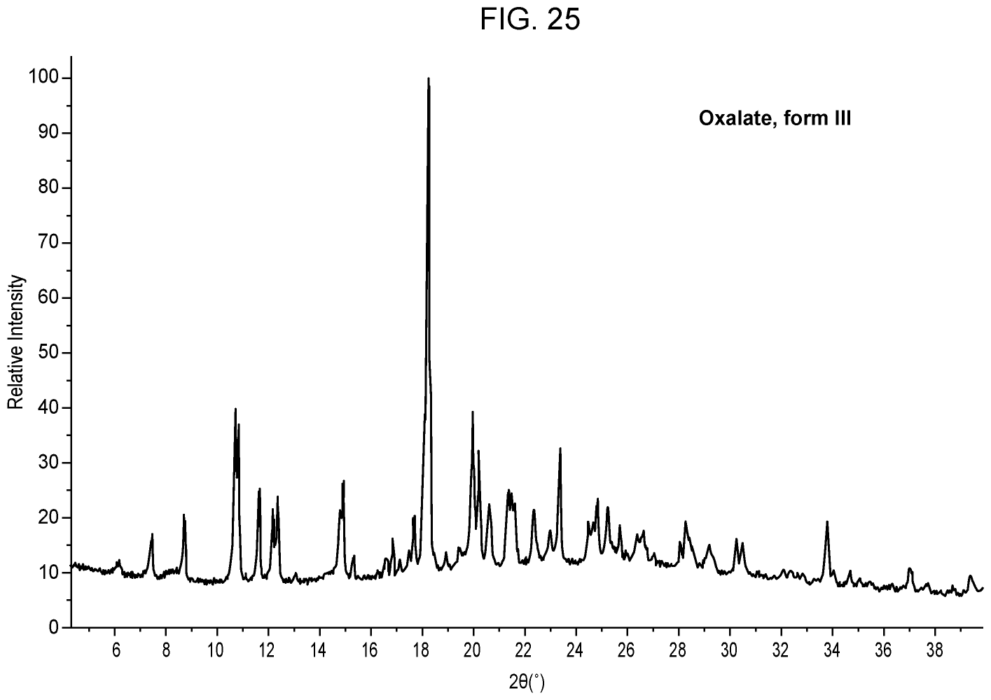

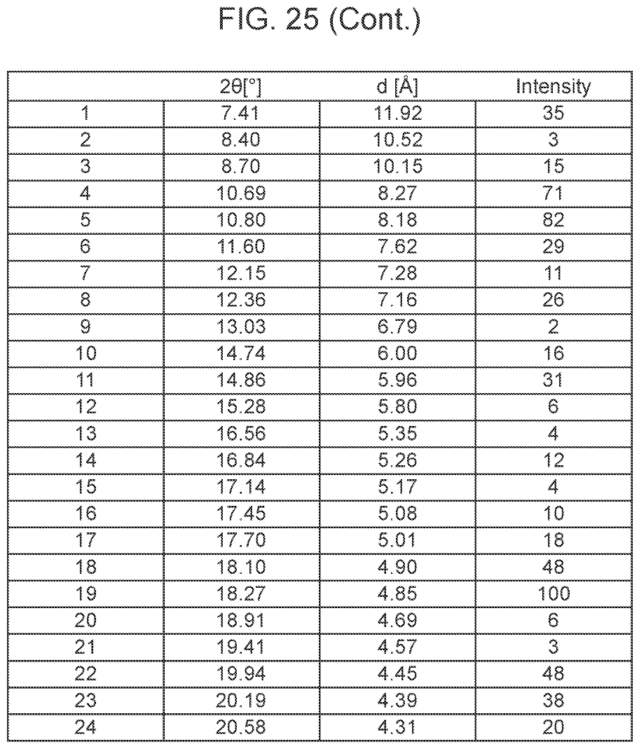

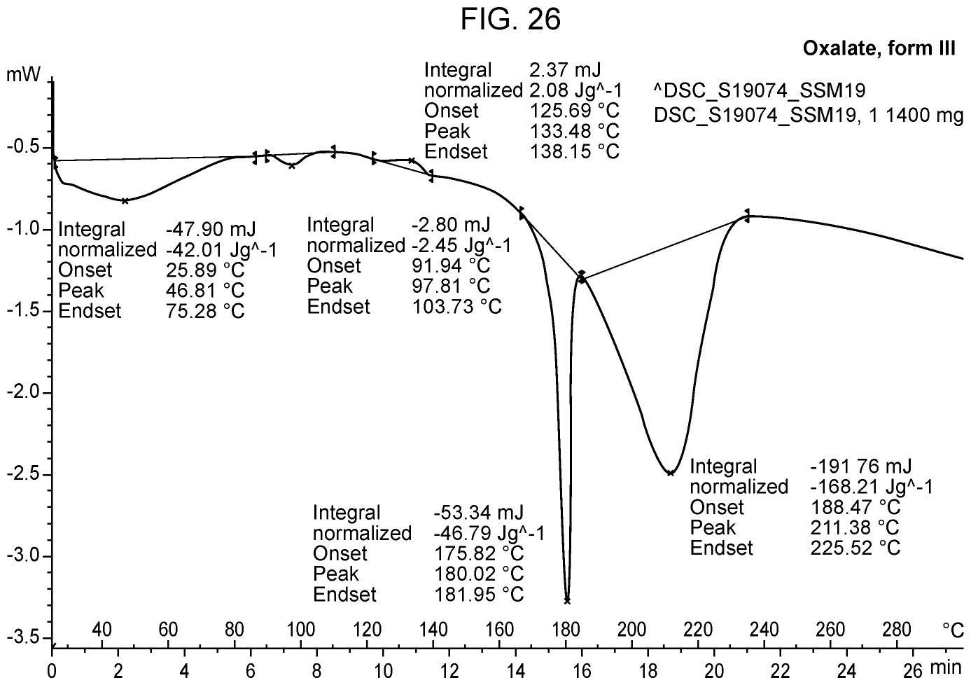

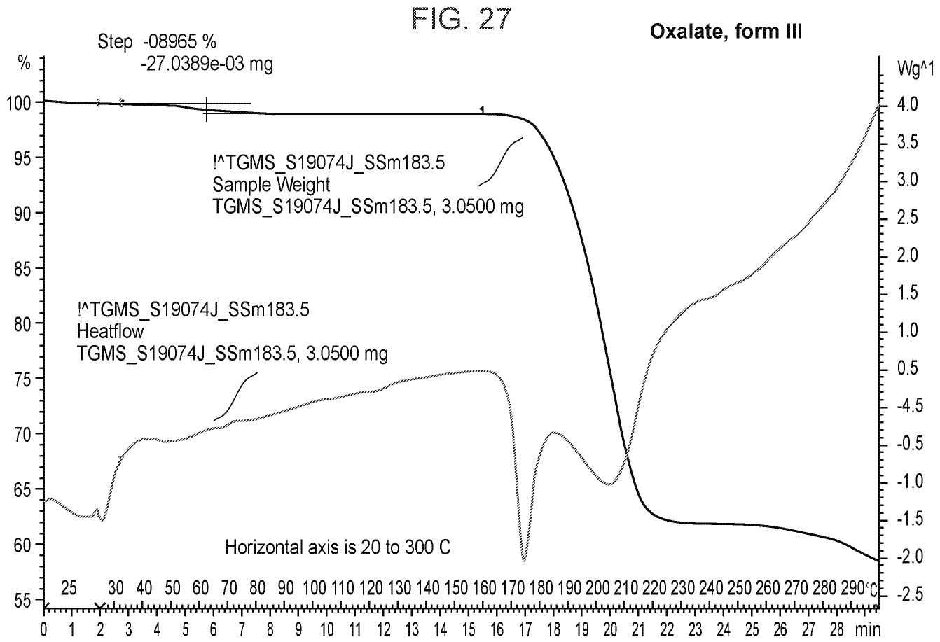

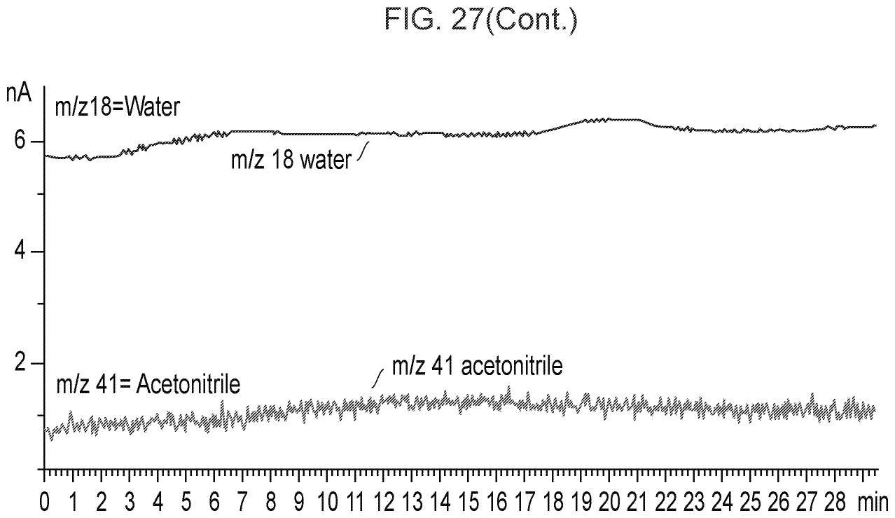

35. The polymorph of clause 34, wherein the polymorph has form I.
36. The polymorph of clause 35, characterized by an XRD pattern comprising 2θ peaks, ±0.2 degrees 2θ, at 4.1, 8.1, 12.2, and 19.6.
37. The polymorph of any one of clauses 35-36, characterized by an XRD pattern substantially as shown in FIG. 19.
38. The polymorph of any one of clauses 35-37, characterized by a DSC thermogram substantially as shown in FIG. 20.
39. The polymorph of any one of clauses 35-38, characterized by a TGA-MS spectrum substantially as shown in FIG. 21.
40. The polymorph of clause 34, wherein the polymorph has form II.
41. The polymorph of clause 40, characterized by an XRD pattern comprising 2θ peaks, ±0.2 degrees 2θ, at 6.1, 8.2, 12.3, 19.6, and 22.8.
42. The polymorph of any one of clauses 40-41, characterized by an XRD pattern substantially as shown in FIG. 22.
43. The polymorph of any one of clauses 40-42, characterized by a DSC thermogram substantially as shown in FIG. 23.
44. The polymorph of any one of clauses 40-43, characterized by a TGA-MS spectrum substantially as shown in FIG. 24.
45. The polymorph of clause 34, wherein the polymorph has form III.
46. The polymorph of clause 45, characterized by an XRD pattern comprising 2θ peaks, ±0.2 degrees 2θ, at 10.8, 18.3, and 19.9.
47. The polymorph of any one of clauses 45-46, characterized by an XRD pattern substantially as shown in FIG. 25.
48. The polymorph of any one of clauses 45-47, characterized by a DSC thermogram substantially as shown in FIG. 26.
49. The polymorph of any one of clauses 45-48, characterized by a TGA-MS spectrum substantially as shown in FIG. 27.
50. A polymorph of a fumarate salt of formula (I):

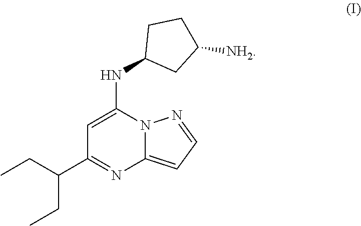 [SMILES: CCC(CC)c1cc(N[C@H]2CC[C@H](N)C2)n2nccc2n1]

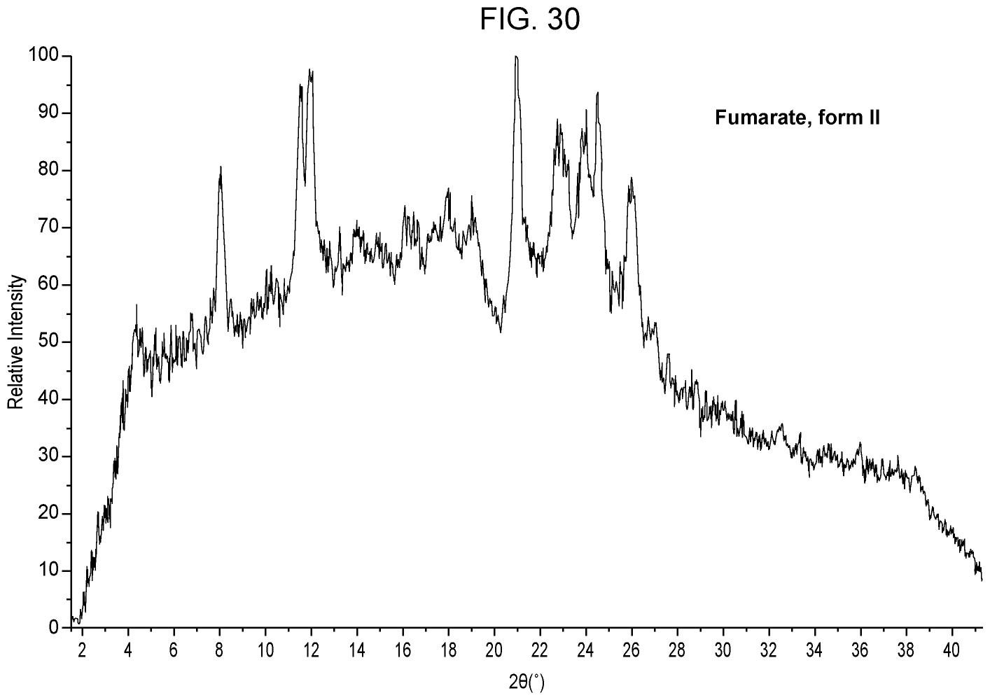

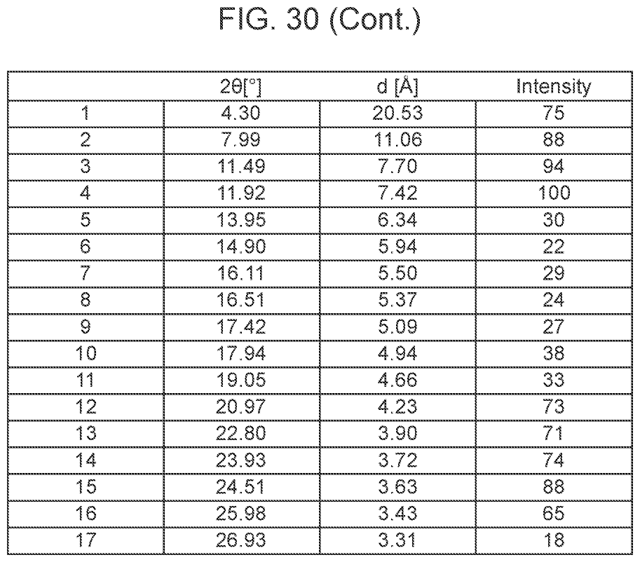

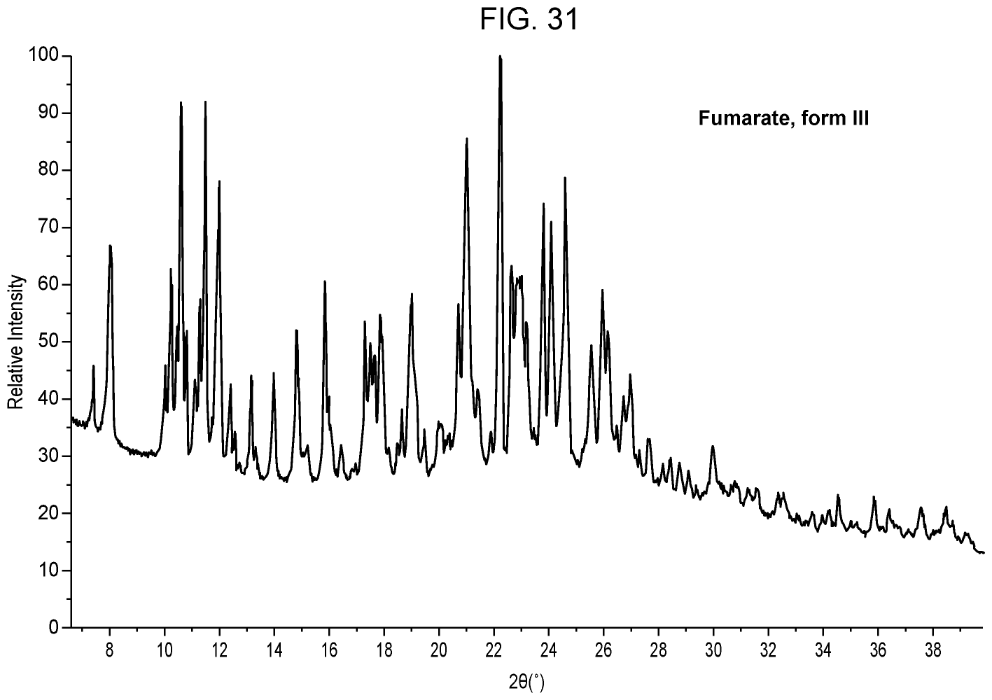

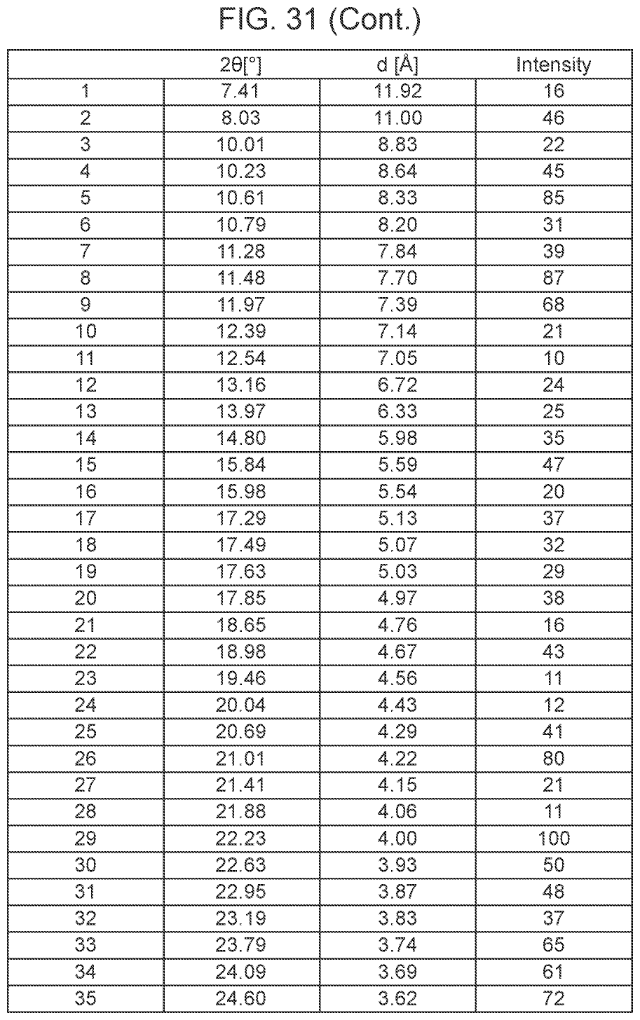

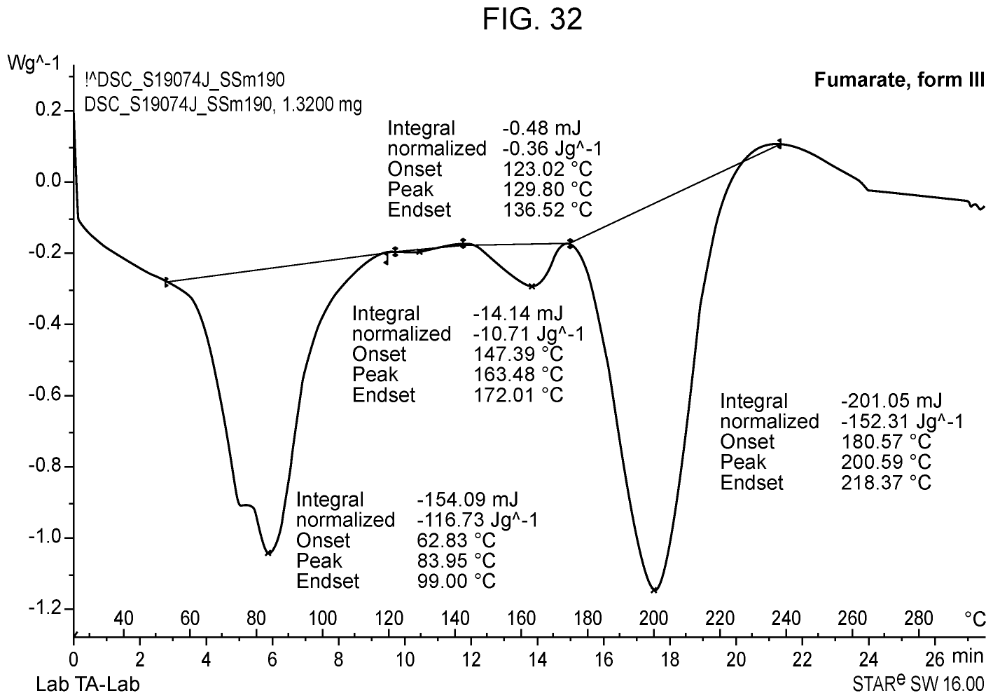

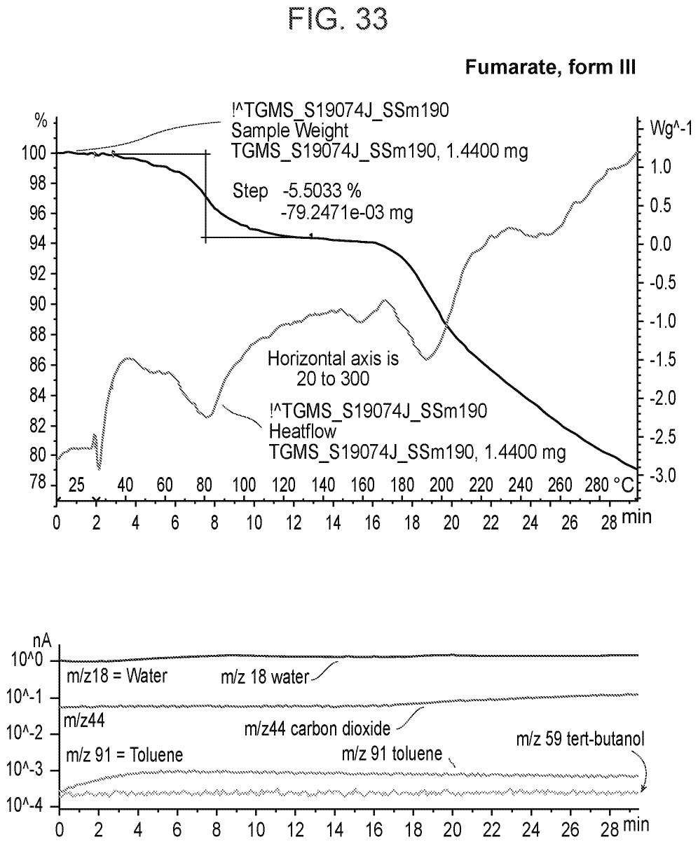

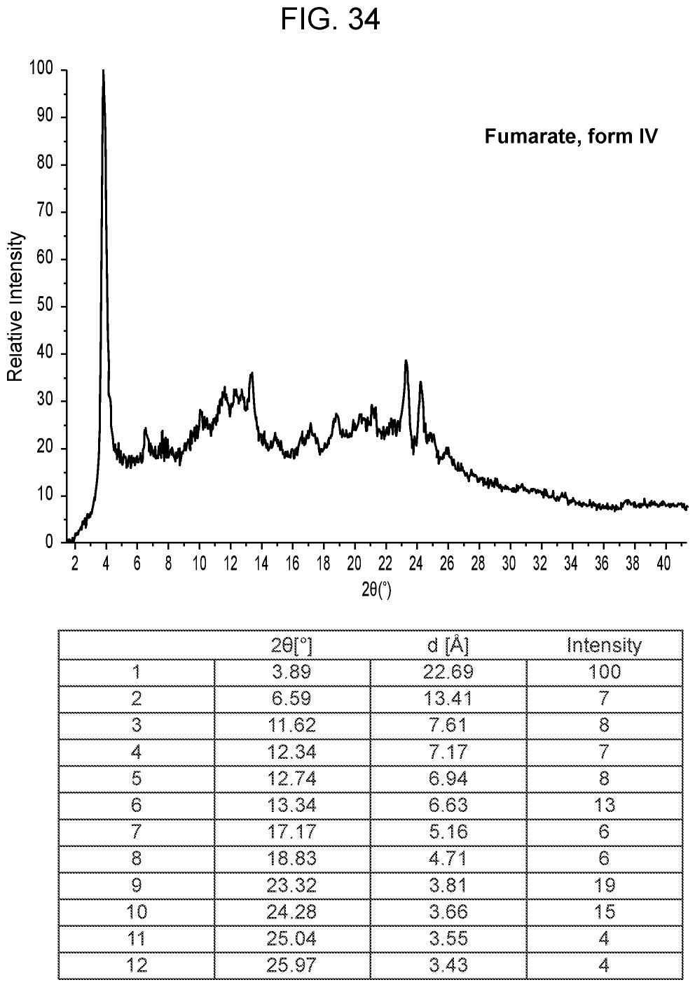

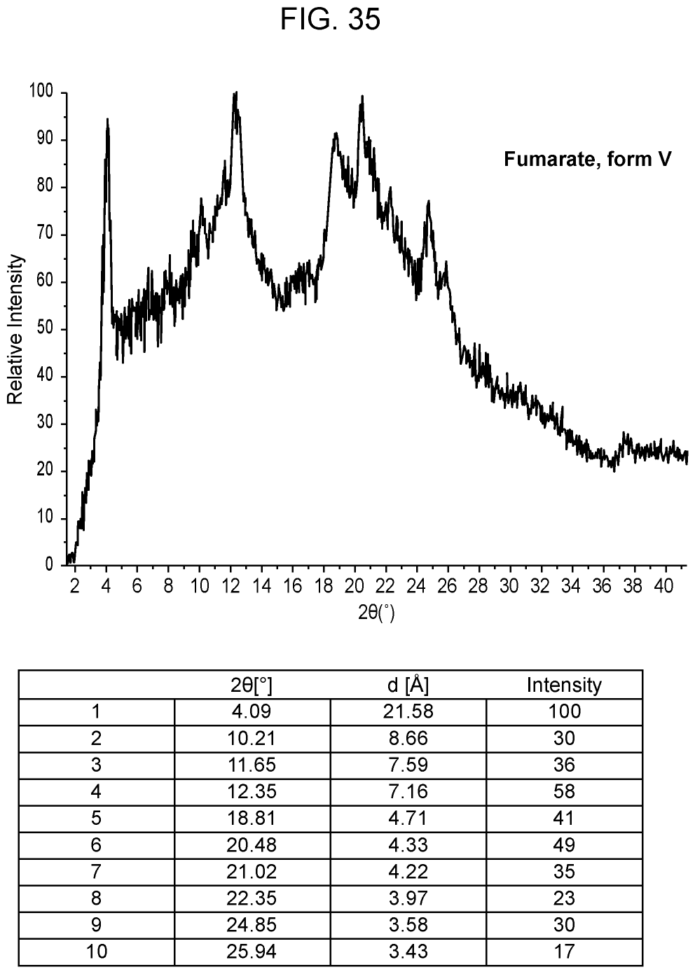

51. The polymorph of clause 50, wherein the polymorph has form I.
52. The polymorph of clause 51, characterized by an XRD pattern comprising 2θ peaks, ±0.2 degrees 2θ, at 4.2, 12.7, 18.7, and 24.7.
53. The polymorph of any one of clauses 51-52, characterized by an XRD pattern substantially as shown in FIG. 29.
54. The polymorph of clause 50, wherein the polymorph has form II.
55. The polymorph of clause 54, characterized by an XRD pattern comprising 2θ peaks, ±0.2 degrees 2θ, at 4.3, 8.0, 11.5, 11.9, and 24.5.
56. The polymorph of any one of clauses 54-55, characterized by an XRD pattern substantially as shown in FIG. 30.
57. The polymorph of clause 50, wherein the polymorph has form III.
58. The polymorph of clause 57, characterized by an XRD pattern comprising 2θ peaks, ±0.2 degrees 2θ, at 10.6, 11.5, 21.0 22.2, and 24.6.
59. The polymorph of any one of clauses 57-58, characterized by an XRD pattern substantially as shown in FIG. 31.
60. The polymorph of any one of clauses 57-59, characterized by a DSC thermogram substantially as shown in FIG. 32.
61. The polymorph of any one of clauses 57-60, characterized by a TGA-MS spectrum substantially as shown in FIG. 33.
62. The polymorph of clause 50, wherein the polymorph has form IV.
63. The polymorph of clause 62, characterized by an XRD pattern comprising 2θ peaks, ±0.2 degrees 2θ, at 3.9, 13.3, 23.3, and 24.3.
64. The polymorph of any one of clauses 62-63, characterized by an XRD pattern substantially as shown in FIG. 34.
65. The polymorph of clause 50, wherein the polymorph has form V.
66. The polymorph of clause 65, characterized by an XRD pattern comprising 2θ peaks, ±0.2 degrees 2θ, at 4.1, 12.4, 20.5, and 21.0.
67. The polymorph of any one of clauses 65-66, characterized by an XRD pattern substantially as shown in FIG. 35.
68. A polymorph of a camsylate salt of formula (I):

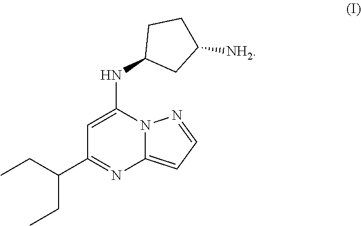 [SMILES: CCC(CC)c1cc(N[C@H]2CC[C@H](N)C2)n2nccc2n1]

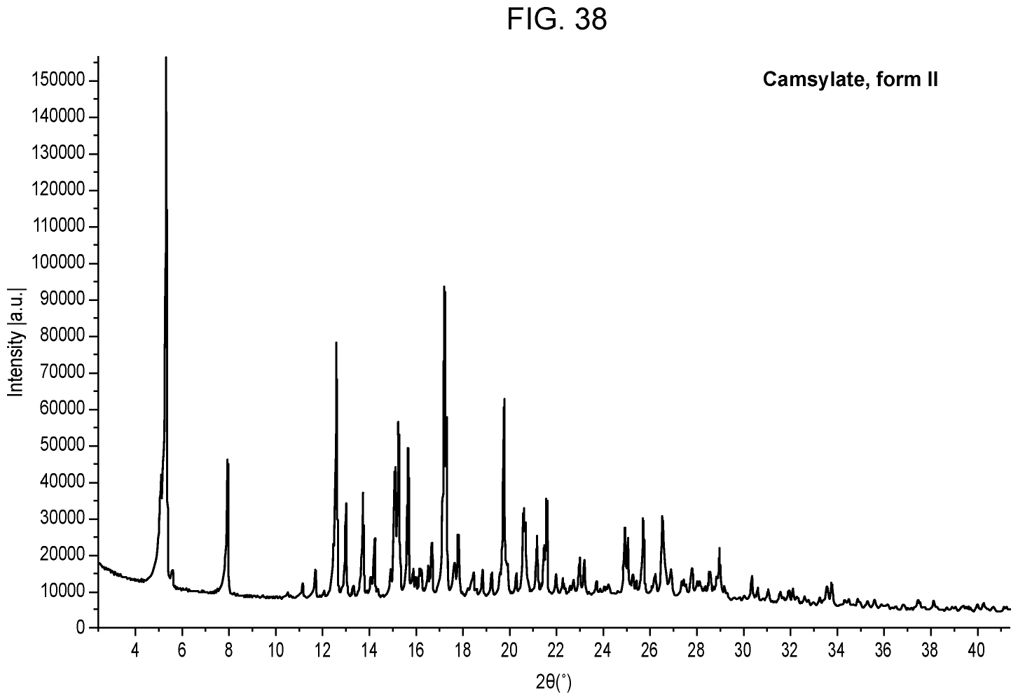

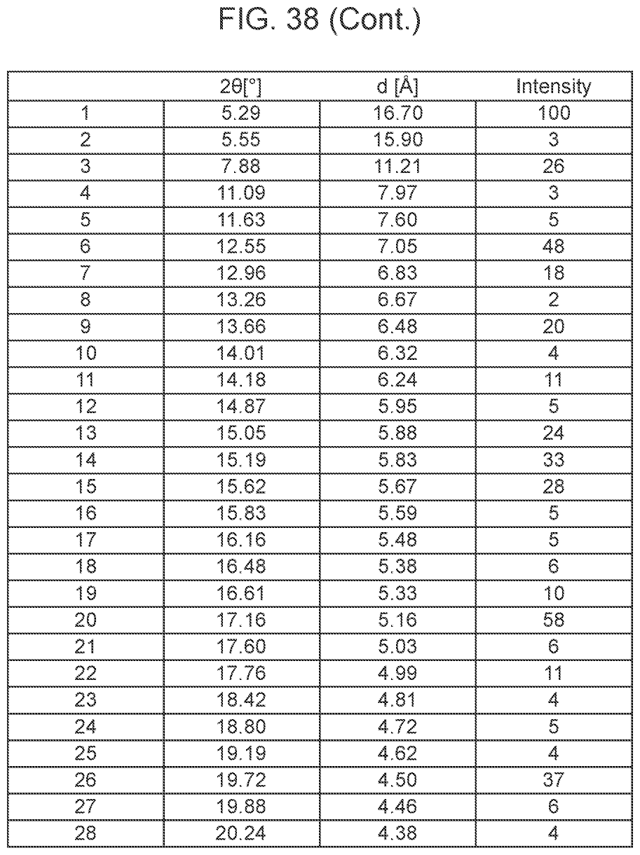

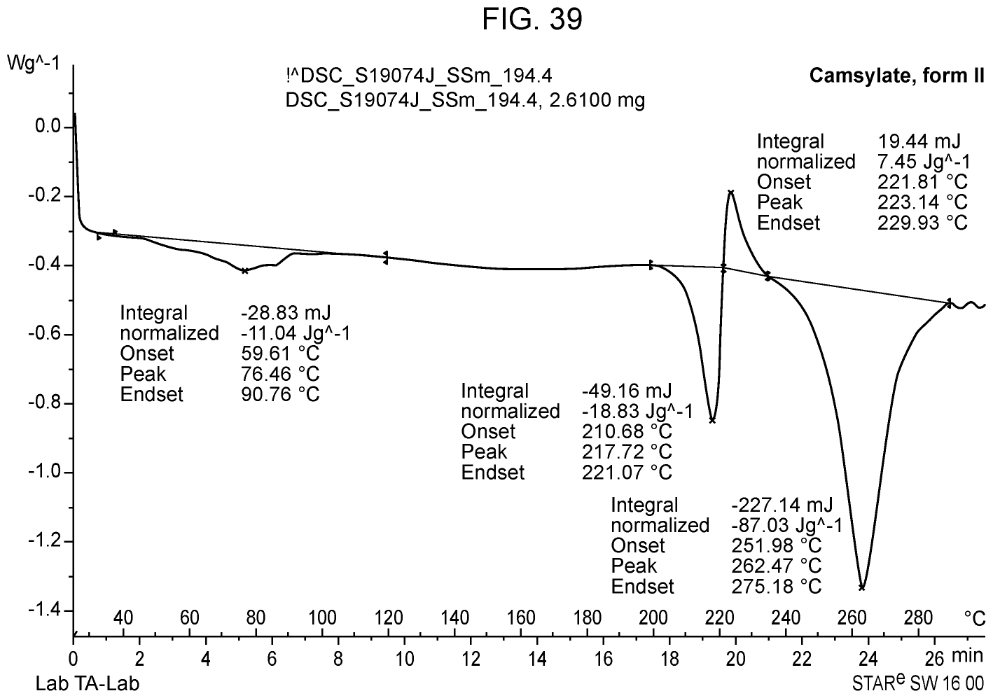

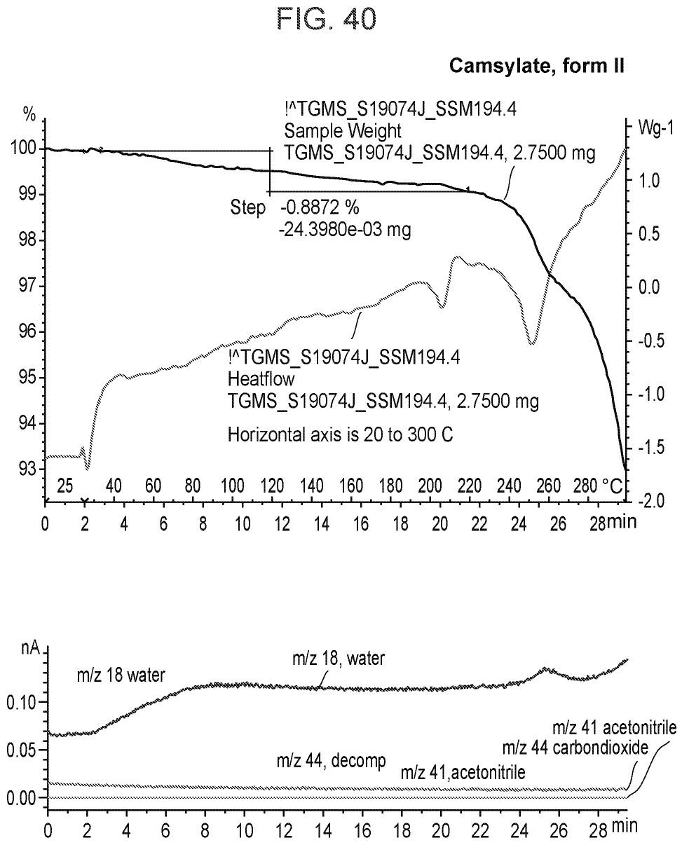

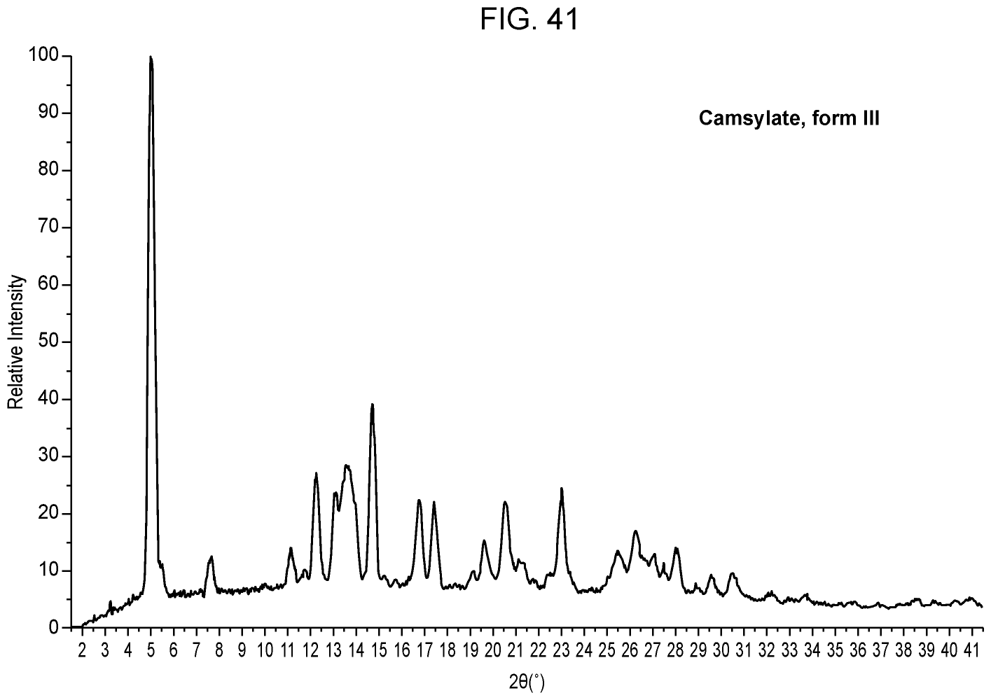

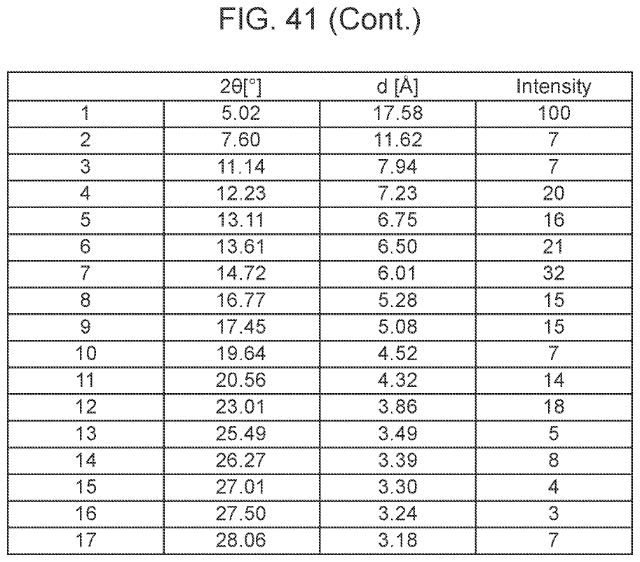

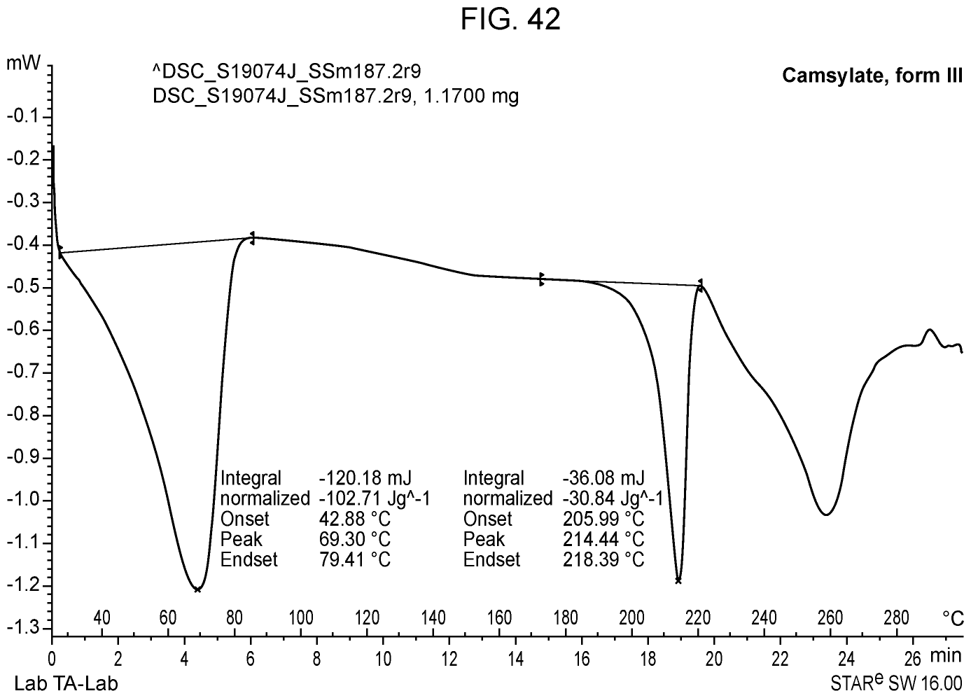

69. The polymorph of clause 68, wherein the polymorph has form I.
70. The polymorph of clause 69, characterized by an XRD pattern comprising 2θ peaks, ±0.2 degrees 2θ, at 4.8, 14.5, 16.8, 17.5, and 20.2.
71. The polymorph of any one of clauses 69-70, characterized by an XRD pattern substantially as shown in FIG. 37.
72. The polymorph of clause 68, wherein the polymorph has form II.
73. The polymorph of clause 72, characterized by an XRD pattern comprising 2θ peaks, ±0.2 degrees 2θ, at 5.3, 12.6, 15.2, 17.2, and 19.7.
74. The polymorph of any one of clauses 72-73, characterized by an XRD pattern substantially as shown in FIG. 38.
75. The polymorph of any one of clauses 72-74, characterized by a DSC thermogram substantially as shown in FIG. 39.
76. The polymorph of any one of clauses 72-75, characterized by a TGA-MS spectrum substantially as shown in FIG. 40.
77. The polymorph of clause 68, wherein the polymorph has form III.
78. The polymorph of clause 77, characterized by an XRD pattern comprising θ peaks, ±0.2 degrees 2θ, at 5.0, 12.2, 14.7, and 23.0.
79. The polymorph of any one of clauses 77-78, characterized by an XRD pattern substantially as shown in FIG. 41.
80. The polymorph of any one of clauses 77-79, characterized by a DSC thermogram substantially as shown in FIG. 42.
81. A polymorph of a citrate salt of formula (I):

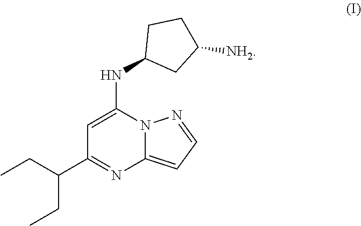 [SMILES: CCC(CC)c1cc(N[C@H]2CC[C@H](N)C2)n2nccc2n1]

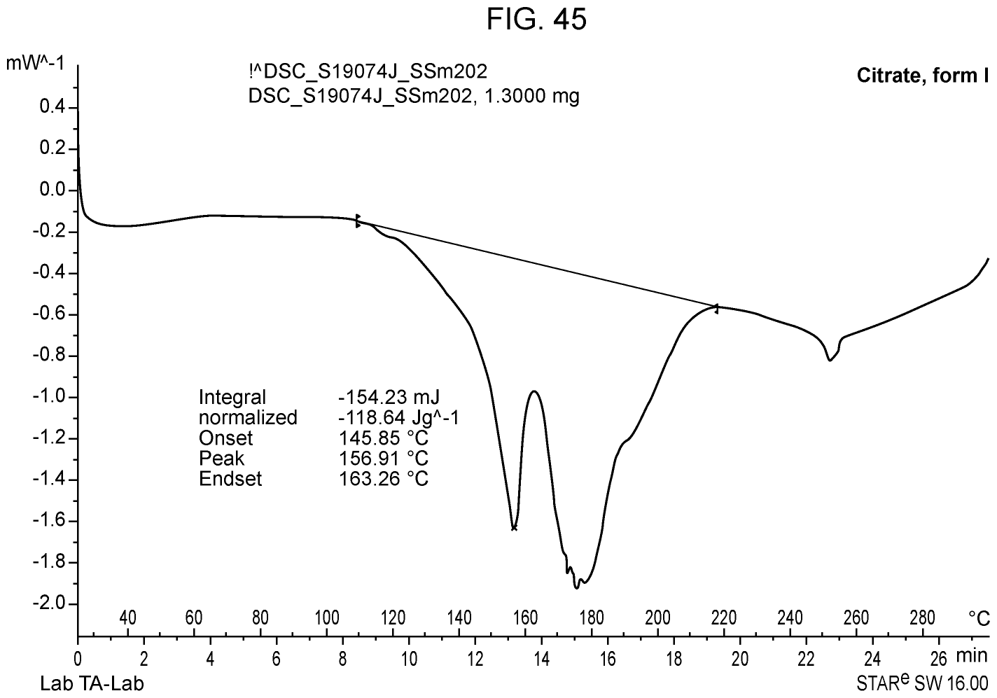

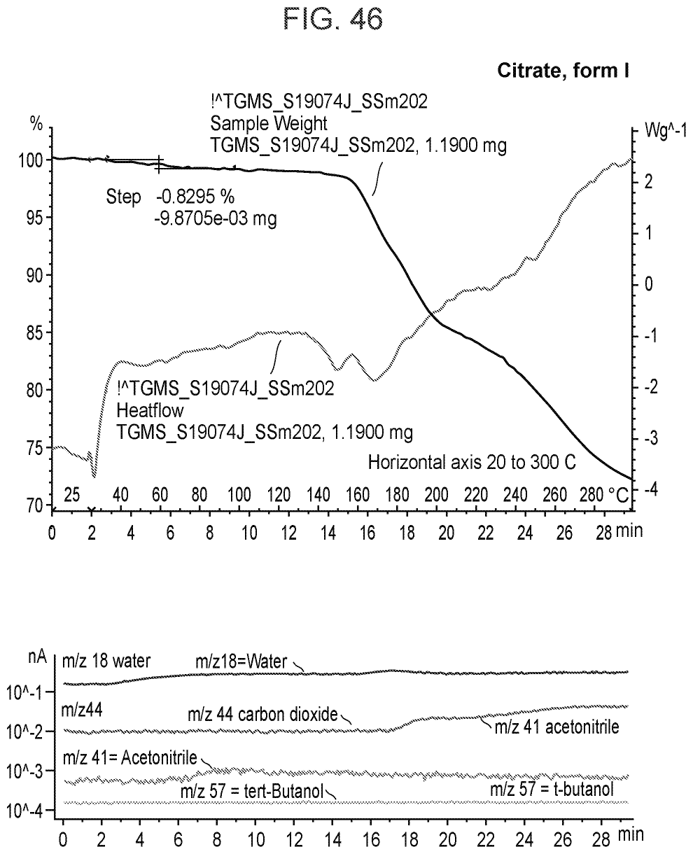

82. The polymorph of clause 81, wherein the polymorph has form I.
83. The polymorph of clause 82, characterized by an XRD pattern comprising 2θ peaks, ±0.2 degrees 2θ, at 4.3, 8.0, 13.3, and 20.8.
84. The polymorph of any one of clauses 82-83, characterized by an XRD pattern substantially as shown in FIG. 44.
85. The polymorph of any one of clauses 82-84, characterized by a DSC thermogram substantially as shown in FIG. 45.
86. The polymorph of any one of clauses 82-85, characterized by a TGA-MS spectrum substantially as shown in FIG. 46.
87. A polymorph of a hydrochloride salt of formula (I):

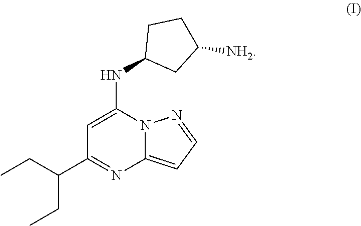 [SMILES: CCC(CC)c1cc(N[C@H]2CC[C@H](N)C2)n2nccc2n1]

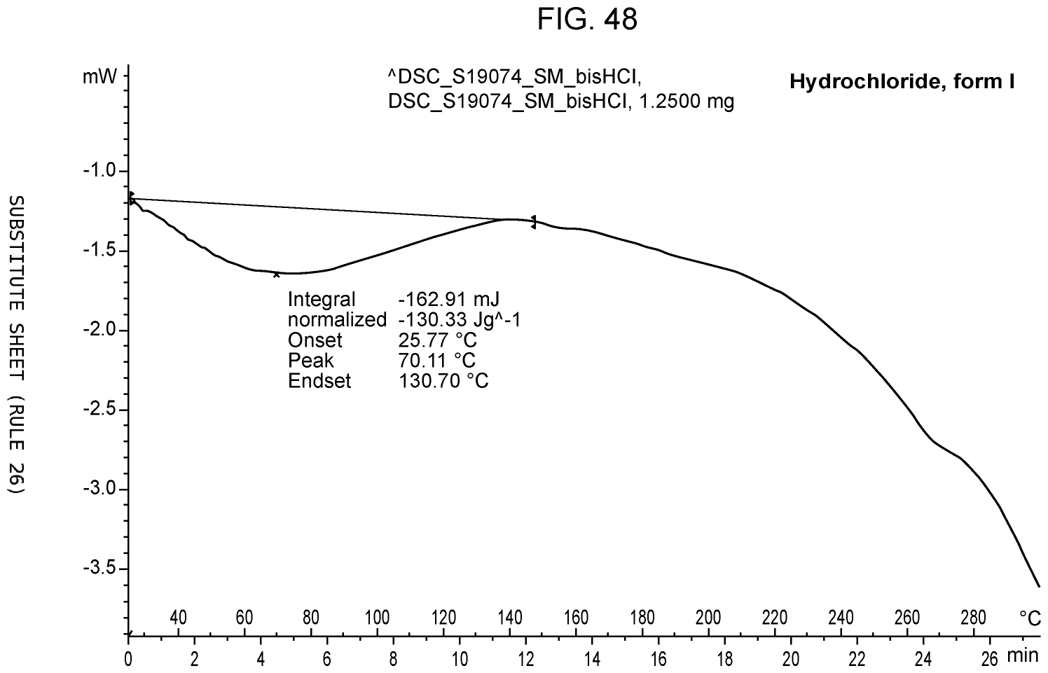

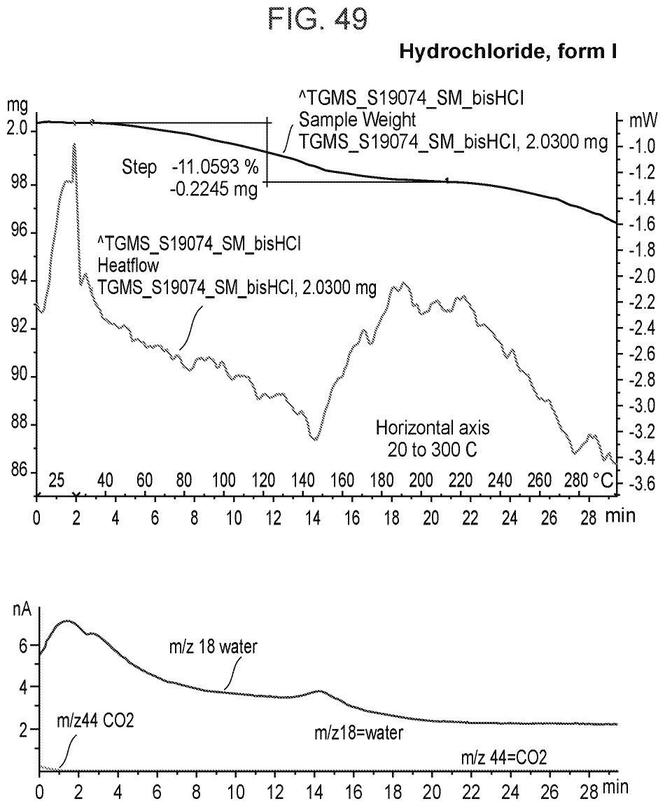

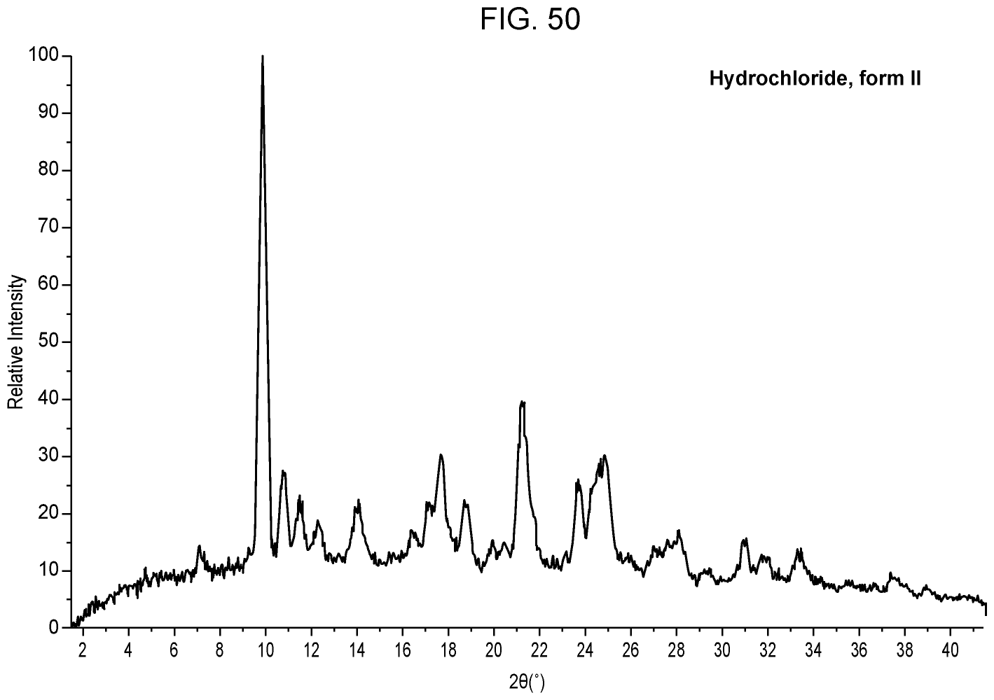

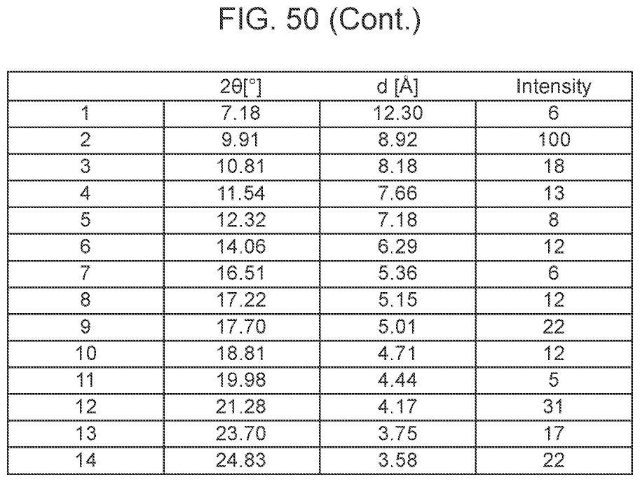

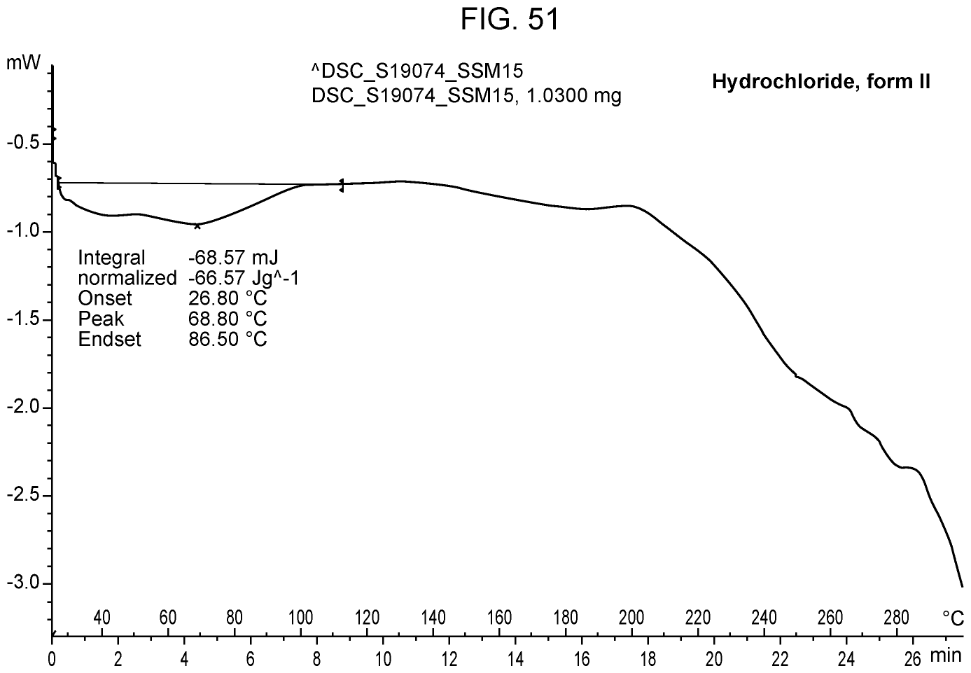

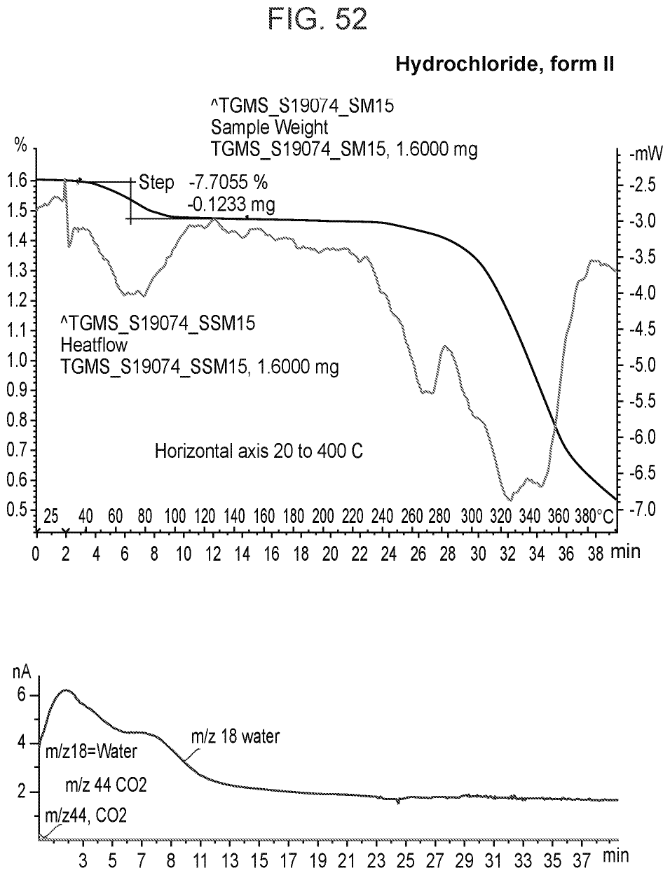

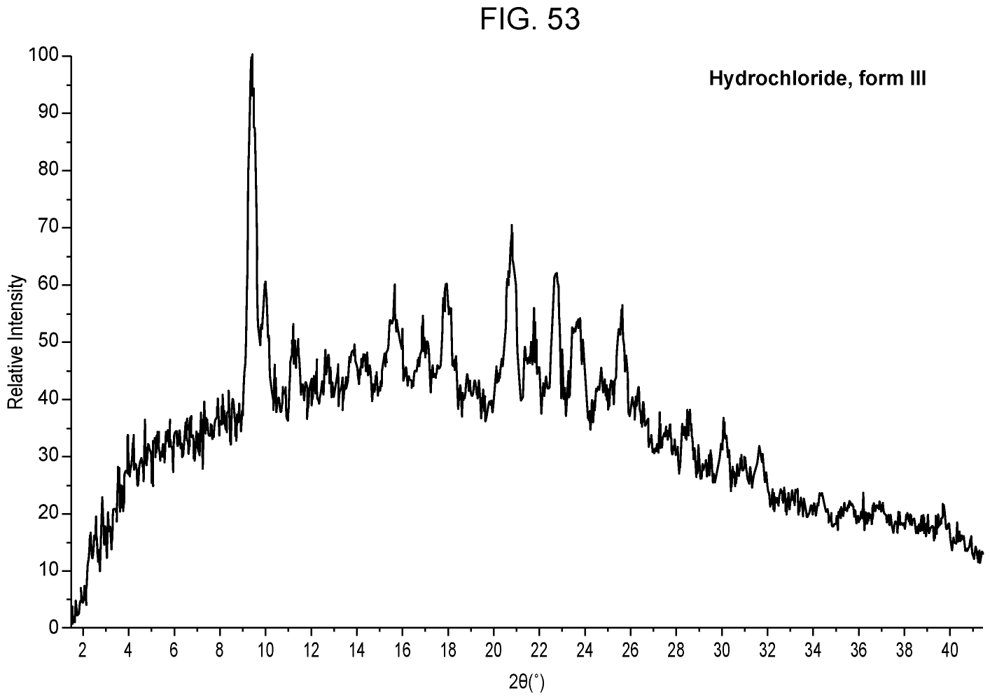

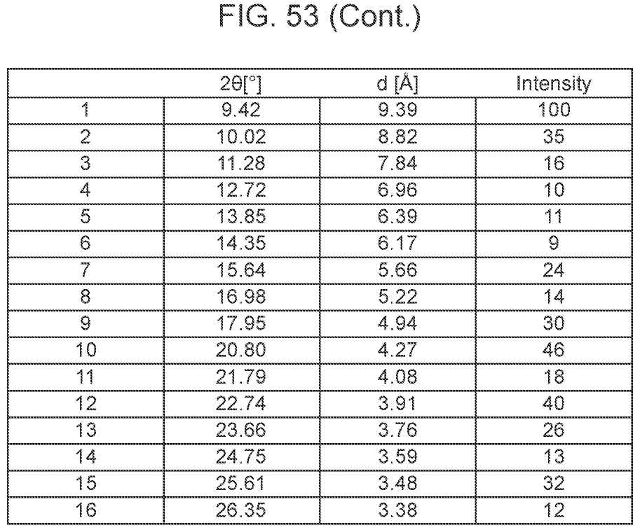

88. The polymorph of clause 87, wherein the polymorph has form I.
89. The polymorph of clause 88, characterized by an amorphous XRD pattern.
90. The polymorph of any one of clauses 87-88, characterized by an XRD pattern substantially as shown in FIG. 47.
91. The polymorph of any one of clauses 87-89, characterized by a DSC thermogram substantially as shown in FIG. 48.
92. The polymorph of any one of clauses 87-90, characterized by a TGA-MS spectrum substantially as shown in FIG. 49.
93. The polymorph of clause 87, wherein the polymorph has form II.
94. The polymorph of clause 93, characterized by an XRD pattern comprising 2θ peaks, ±0.2 degrees 2θ, at 9.9, 17.7, 21.3, and 24.8.
95. The polymorph of any one of clauses 94-95, characterized by an XRD pattern substantially as shown in FIG. 50.
96. The polymorph of any one of clauses 94-96, characterized by a DSC thermogram substantially as shown in FIG. 51.
97. The polymorph of any one of clauses 94-97, characterized by a TGA-MS spectrum substantially as shown in FIG. 52.
98. The polymorph of clause 87, wherein the polymorph has form III.
99. The polymorph of clause 98, characterized by an XRD pattern comprising 2θ peaks, ±0.2 degrees 2θ, at 9.4, 20.8, 22.7, and 25.6.
100. The polymorph of any one of clauses 98-99, characterized by an XRD pattern substantially as shown in FIG. 53.
101. A polymorph of a naphthalene-1,5-disulfonic acid salt of formula (I):

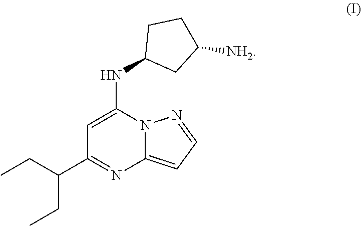 [SMILES: CCC(CC)c1cc(N[C@H]2CC[C@H](N)C2)n2nccc2n1]

102. The polymorph of clause 101, wherein the polymorph has form I.
103. The polymorph of clause 102, characterized by an XRD pattern comprising 2θ peaks, ±0.2 degrees 2θ, at 8.3, 12.7, 13.1, 22.4, and 24.1.
104. The polymorph of any one of clauses 102-103, characterized by an XRD pattern substantially as shown in FIG. 54.
105. A pharmaceutical composition, comprising:
   a polymorph of any one of clauses 1-104; and
   a pharmaceutically acceptable carrier.
106. A kit, comprising:
   a polymorph of any one of clauses 1-104 or a pharmaceutical composition of clause 117; and
   instructions for using the polymorph of any one of clauses 1-104 or the pharmaceutical composition of clause 117.
107. A method of treating a condition in a subject, the method comprising:
   administering to the subject a polymorph of any one of clauses 1-104 or a pharmaceutical composition of clause 105.
108. The method of clause 107, wherein the subject is human.
109. The method of any one of clauses 107-108, wherein the condition is cancer or an autoimmune disease.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims.

1. A polymorph of a free base or a salt of formula (I):

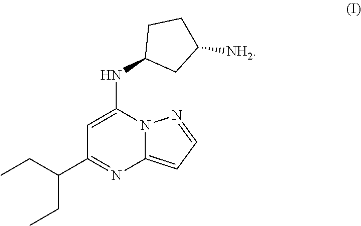 [SMILES: CCC(CC)c1cc(N[C@H]2CC[C@H](N)C2)n2nccc2n1]

2. The polymorph of claim 1, wherein the polymorph of the free base has form I, form II, or form III.

3. The polymorph of claim 1, wherein the polymorph is a tosylate salt of formula (I):

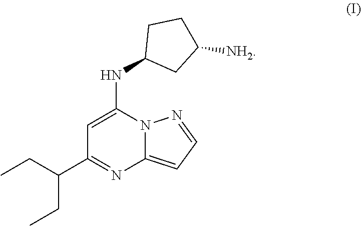 [SMILES: CCC(CC)c1cc(N[C@H]2CC[C@H](N)C2)n2nccc2n1]

4. The polymorph of claim 3, wherein the polymorph has form I, form II, form III, or form IV.

5. The polymorph of claim 1, wherein the polymorph is an oxalate salt of formula (I):

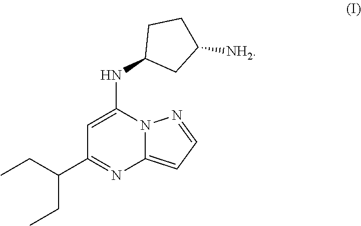 [SMILES: CCC(CC)c1cc(N[C@H]2CC[C@H](N)C2)n2nccc2n1]

6. The polymorph of claim 5, wherein the polymorph has form I, form II, or form III.

7. The polymorph of claim 1, wherein the polymorph is a fumarate salt of formula (I):

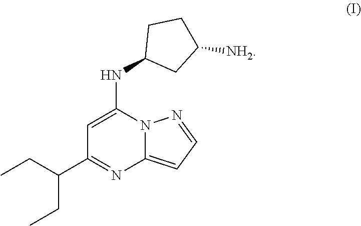 [SMILES: CCC(CC)c1cc(N[C@H]2CC[C@H](N)C2)n2nccc2n1]

8. The polymorph of claim 7, wherein the polymorph has form I, form II, form III, form IV, or form V.

9. The polymorph of claim 1, wherein the polymorph is a camsylate salt of formula (I):

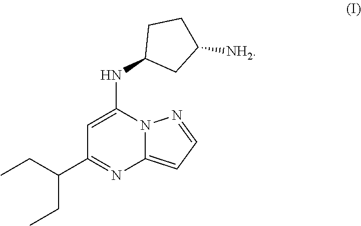 [SMILES: CCC(CC)c1cc(N[C@H]2CC[C@H](N)C2)n2nccc2n1]

10. The polymorph of claim 9, wherein the polymorph has form I, form II, or form III.

11. The polymorph of claim 1, wherein the polymorph is a citrate salt of formula (I):

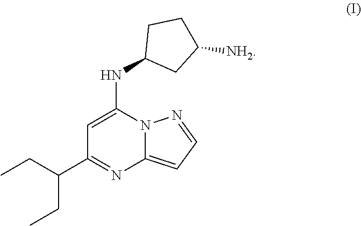 [SMILES: CCC(CC)c1cc(N[C@H]2CC[C@H](N)C2)n2nccc2n1]

12. The polymorph of claim 11, wherein the polymorph has form I.

13. The polymorph of claim 1, wherein the polymorph is a hydrochloride salt of formula (I):

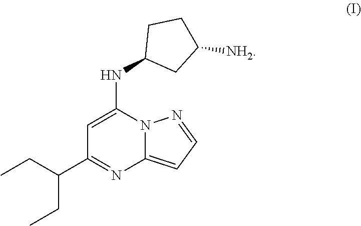 [SMILES: CCC(CC)c1cc(N[C@H]2CC[C@H](N)C2)n2nccc2n1]

14. The polymorph of claim 13, wherein the polymorph has form I, form II, or form III.

15. The polymorph of claim 1, wherein the polymorph is a naphthalene-1,5-disulfonic acid salt of formula (I):

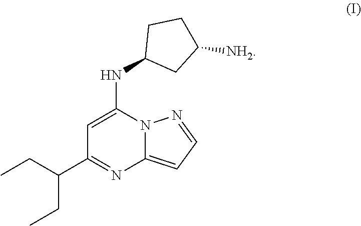 [SMILES: CCC(CC)c1cc(N[C@H]2CC[C@H](N)C2)n2nccc2n1]

16. A pharmaceutical composition, comprising: a polymorph of claim 1; and a pharmaceutically acceptable carrier.

17. A kit, comprising: a polymorph of claim 1; and instructions for using the polymorph of claim 1.

18. A method of treating a condition in a subject, the method comprising: administering to the subject a polymorph of claim 1.

19. The method of claim 18, wherein the subject is human.

20. The method of claim 18, wherein the condition is cancer or an autoimmune disease.
